SEQUENCE LISTING (14 sequences)

SEQ ID NO: 1 - protein, 305 aa, Helicobacter pylori 908
MILTAMQDAIGRTPIFKFTRKDYPIPLKSAIYAKLEHLNPGGSVKDRLGQYLIKEAFRTHKITSTTTIIEPTAGNTGIALALVAIKHHLKTIFVVPEKFSVEKQQIMRALGALVINTPTSEGISGAIKKSKELAESIPDSYLPLQFENPDNPAAYYHTLAPEIVKELGTNFTSFVAGIGSGGTFAGTAKYLKERIPNIRLIGVEPEGSILNGGEPGPHEIEGIGVEFIPPFFANLDIDGFETISDEEGFSYTRKLAKKNGLLVGSSSGAAFAAALKEVQRLPEGSQVLTIFPDMADRYLSKGIYS

SEQ ID NO: 2 - DNA, 1215 bp, Artificial Sequence
  synthetic construct - A nucleotide encoding an optimized cystathionine beta synthase (oCBS). oCBS is constructed with codon usage enabling high E. coli expression and the addition of a cellulose binding domain for ease of purification with cellulose.
atgaccccggtgtctggcaacctgaaagtcgaattttacaactccaatccgtctgataccacgaatagcattaacccgcagttcaaagttacgaacaccggcagctctgcgattgatctgtcaaaactgacgctgcgttattactataccgtcgatggtcagaaagaccaaaccttttggtgcgaccatgcggccattatcggtagtaacggctcctacaatggcattacgtctaatgtcaaaggcaccttcgtgaaaatgagttcctcaacgaacaatggcgccggtgcaggcgctatgatcctgaccgcgatgcaggatgccatcggccgtacgccgatttttaaattcacccgcaaagactacccgatcccgctgaaaagtgcaatttatgctaaactggaacatctgaatccgggcggcagcgtgaaagatcgtctgggtcaatatctgattaaagaagcctttcgcacgcacaaaatcaccagcaccacgaccattatcgaaccgacggcgggtaataccggtatcgcactggccctggttgccattaaacatcacctgaaaaccatctttgtggttccggaaaaattctcagtcgaaaaacagcaaatcatgcgtgcgctgggcgccctggtgatcaacacgccgacctcagaaggtatctcgggcgcaattaaaaaatcgaaagaactggctgaaagcattccggattcttacctgccgctgcaatttgaaaacccggacaatccggcagcttactatcataccctggcaccggaaattgtgaaagaactgggcacgaattttaccagcttcgttgctggtatcggctctggcggtacgttcgcaggcaccgctaaatatctgaaagaacgtattccgaacatccgcctgattggcgtggaaccggaaggtagtattctgaatggcggtgaaccgggtccgcacgaaatcgaaggtattggcgttgaatttatcccgccgtttttcgccaacctggatattgacggctttgaaacgatttcagatgaagaaggtttctcgtatacccgcaaactggcgaagaaaaacggtctgctggttggcagcagcagcggtgcagcatttgcagctgcgctgaaagaagttcagcgtctgccggaaggcagccaagtcctgaccattttcccggatatggcggaccgctacctgagtaaaggtatctattcctaa

SEQ ID NO: 3 - DNA, 18 bp, Artificial Sequence
  synthetic construct - A nucleotide encoding a linker between the cellulose binding domain and the optimized cystathionine beta synthase.
ggcgccggtgcaggcgct

SEQ ID NO: 4 - DNA, 279 bp, Artificial Sequence
  synthetic construct -  A nucleotide encoding a cellulose binding domain.
atgaccccggtgtctggcaacctgaaagtcgaattttacaactccaatccgtctgataccacgaatagcattaacccgcagttcaaagttacgaacaccggcagctctgcgattgatctgtcaaaactgacgctgcgttattactataccgtcgatggtcagaaagaccaaaccttttggtgcgaccatgcggccattatcggtagtaacggctcctacaatggcattacgtctaatgtcaaaggcaccttcgtgaaaatgagttcctcaacgaacaat

SEQ ID NO: 5 - protein, 404 aa, Artificial Sequence
  synthetic construct - The protein sequence of an optimized cystathionine beta synthase (oCBS). oCBS is constructed with codon usage enabling high E. coli expression and the addition of a cellulose binding domain for ease of purification with cellulose.
MTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNGAGAGAMILTAMQDAIGRTPIFKFTRKDYPIPLKSAIYAKLEHLNPGGSVKDRLGQYLIKEAFRTHKITSTTTIIEPTAGNTGIALALVAIKHHLKTIFVVPEKFSVEKQQIMRALGALVINTPTSEGISGAIKKSKELAESIPDSYLPLQFENPDNPAAYYHTLAPEIVKELGTNFTSFVAGIGSGGTFAGTAKYLKERIPNIRLIGVEPEGSILNGGEPGPHEIEGIGVEFIPPFFANLDIDGFETISDEEGFSYTRKLAKKNGLLVGSSSGAAFAAALKEVQRLPEGSQVLTIFPDMADRYLSKGIYS

SEQ ID NO: 6 - protein, 6 aa, Artificial Sequence
  synthetic construct - The protein sequence of a linker between the cellulose binding domain and the optimized cystathionine beta synthase.
GAGAGA

SEQ ID NO: 7 - protein, 93 aa, Artificial Sequence
  synthetic construct - The protein sequence of a cellulose binding domain.
MTPVSGNLKVEFYNSNPSDTTNSINPQFKVTNTGSSAIDLSKLTLRYYYTVDGQKDQTFWCDHAAIIGSNGSYNGITSNVKGTFVKMSSSTNN

SEQ ID NO: 8 - protein, 378 aa, Helicobacter pylori 908
MQTKLIHGGISEDATTGAVSVPIYQASTYRQDAIGRHKGYEYSRSGNPTRFALEELIADLEGGVKGFAFASGLAGIHAVFSLLQSGDHVLLGDDVYGGTFRLFNKVLVKNGLSCTIIDTSDISQIKKAIKPNTKALYLETPSNPLLKITDLAQCASVAKDHGLLTIVDNTFATPYCQNPLLLGADIVAHSGTKYLGGHSDVVAGLVTTNNEALAQEIAFFQNAIGGVLGPQDSWLLQRGIKTLGLRMEAHQKNALCVAEFLEKHPKVERVYYPGLPTHPNHELAKAQMRGFSGMLSFTLKNDSEAALFVESLKLFILGESLGGVESLVGIPALMTHACIPKEQREAAGIRDGLVRLSVGIEHEQDLLEDLEQAFAKIS

SEQ ID NO: 9 - DNA, 1434 bp, Artificial Sequence
  synthetic construct - A nucleotide encoding an optimized cystathionine gamma lyase (oCGL). oCGL is constructed with codon usage enabling high E. coli expression and the addition of a cellulose binding domain for ease of purification with cellulose.
atgacgccggtgtctggcaatctgaaagtggaattttacaacagcaacccgagcgatacgacgaatagcatcaacccgcagttcaaagtgaccaacacgggtagctctgcgattgatctgtctaaactgaccctgcgttattactatacggttgatggccagaaagaccaaaccttttggtgcgaccatgcggccattatcggttctaacggcagttataatggtatcaccagcaatgtgaaaggcacgttcgttaaaatgagttcctcaaccaacaatggcgcaggtgctggcgcgatgcagacgaaactgattcatggcggtatcagcgaagatgcaaccacgggtgcagtctcggtgccgatttaccaggccagcacctatcgtcaagacgcaatcggtcgccacaaaggctacgaatattcgcgtagcggtaacccgacgcgctttgcactggaagaactgattgcggatctggaaggcggtgtgaaaggctttgccttcgcatcaggtctggcaggcatccatgctgttttctcgctgctgcaaagcggtgaccacgtcctgctgggcgatgacgtgtacggcggcacctttcgcctgttcaacaaagttctggtcaaaaatggtctgagttgtaccattatcgatacgtccgacatttcacagatcaaaaaagcgattaaaccgaacaccaaagccctgtatctggaaacgccgtcgaatccgctgctgaaaattaccgatctggcccagtgcgcaagcgttgctaaagatcatggcctgctgacgatcgtggataacacctttgcgacgccgtactgtcaaaatccgctgctgctgggtgcggatattgtcgcccattccggcaccaaatatctgggcggtcactcagacgtggttgccggtctggttaccacgaacaatgaagctctggcgcaggaaattgcgtttttccaaaacgcaatcggcggtgtgctgggtccgcaggatagctggctgctgcaacgtggtatcaaaaccctgggcctgcgcatggaagcgcatcagaaaaatgcactgtgcgttgctgaatttctggaaaaacacccgaaagtggaacgtgtttactatccgggtctgccgacccatccgaaccacgaactggccaaagcacaaatgcgcggtttttctggcatgctgagtttcacgctgaaaaatgattctgaagcagctctgtttgtggaaagtctgaaactgttcattctgggtgaatccctgggcggtgtcgaatcactggtgggcattccggcactgatgacccatgcttgtatcccgaaagaacagcgtgaagcggccggtattcgtgatggcctggttcgcctgtctgtcggcatcgaacacgaacaggatctgctggaagacctggaacaggcgtttgcgaaaattagttaa

SEQ ID NO: 10 - DNA, 18 bp, Artificial Sequence
  synthetic construct - A nucleotide encoding a linker between the cellulose binding domain and the optimized cystathionine gamma lyase.
ggcgcaggtgctggcgcg

SEQ ID NO: 11 - DNA, 279 bp, Artificial Sequence
  synthetic construct - A nucleotide encoding a cellulose binding domain.
atgacgccggtgtctggcaatctgaaagtggaattttacaacagcaacccgagcgatacgacgaatagcatcaacccgcagttcaaagtgaccaacacgggtagctctgcgattgatctgtctaaactgaccctgcgttattactatacggttgatggccagaaagaccaaaccttttggtgcgaccatgcggccattatcggttctaacggcagttataatggtatcaccagcaatgtgaaaggcacgttcgttaaaatgagttcctcaaccaacaat

SEQ ID NO: 12 - protein, 477 aa, Artificial Sequence
  synthetic contruct - The protein sequence of an optimized cystathionine gamma lyase (oCGL). oCGL is constructed with codon usage enabling high E. coli expression and the addition of a cellulose binding domain for ease of purification with cellulose.
MVSYKCGVKDGTKNTIRATINIKNTGTTPVNLSDIKVRYWFTSDGENNFVCDYAAFGTDKVKKKIENSVPGADTYCEISVKGTFVKMSSSTNNGAGAGAMQTKLIHGGISEDATTGAVSVPIYQASTYRQDAIGRHKGYEYSRSGNPTRFALEELIADLEGGVKGFAFASGLAGIHAVFSLLQSGDHVLLGDDVYGGTFRLFNKVLVKNGLSCTIIDTSDISQIKKAIKPNTKALYLETPSNPLLKITDLAQCASVAKDHGLLTIVDNTFATPYCQNPLLLGADIVAHSGTKYLGGHSDVVAGLVTTNNEALAQEIAFFQNAIGGVLGPQDSWLLQRGIKTLGLRMEAHQKNALCVAEFLEKHPKVERVYYPGLPTHPNHELAKAQMRGFSGMLSFTLKNDSEAALFVESLKLFILGESLGGVESLVGIPALMTHACIPKEQREAAGIRDGLVRLSVGIEHEQDLLEDLEQAFAKIS

SEQ ID NO: 13 - protein, 6 aa, Artificial Sequence
  synthetic construct - The protein sequence of a linker between the cellulose binding domain and the optimized cystathionine gamma lyase.
GAGAGA

SEQ ID NO: 14 - protein, 93 aa, Artificial Sequence
  synthetic contruct - The protein sequence of a cellulose binding domain.
MVSYKCGVKDGTKNTIRATINIKNTGTTPVNLSDIKVRYWFTSDGENNFVCDYAAFGTDKVKKKIENSVPGADTYCEISVKGTFVKMSSSTNN

(19) United States
(12) Patent Application Publication
(10) Pub. No.: US 2015/0204882 A1
(43) Pub. Date: Jul. 23, 2015

(54) METHIONINE METABOLITES PREDICT AGGRESSIVE CANCER PROGRESSION

(71) Applicant: CEDARS-SINAI MEDICAL CENTER, Los Angeles, CA (US)

(72) Inventors: Neil A. Bhowmick, Beverly Hills, CA (US); Diptiman Choudhury, Los Angeles, CA (US)

(73) Assignee: CEDARS-SINAI MEDICAL CENTER, Los Angeles, CA (US)

(21) Appl. No.: 14/617,016

(22) Filed: Feb. 9, 2015

Related U.S. Application Data
(63) Continuation-in-part of application No. PCT/US2013/054415, filed on Aug. 9, 2013.
(60) Provisional application No. 61/682,155, filed on Aug. 10, 2012.
(60) Provisional application No. 62/021,648, filed on Jul. 7, 2014.

(51) Int. Cl. G01N 33/68; G01N 27/327; C12Q 1/00; C12N 9/88

(52) U.S. Cl. CPC G01N 33/6815; C12N 9/88; G01N 27/3271; C12Q 1/005; C12Y 402/01022; C12Y 404/01001; G01N 2800/54; G01N 2333/90

(57) ABSTRACT

The invention relates to the use of enzymes, nanorods, and nanoelectronic devices to detect cysteine level in a patient sample and relates to the use of the detected cysteine level to predict cancer recurrence in the patient and to prescribe and/or administer an appropriate therapy to a subject. The invention is directed to systems and methods of detecting cysteine level in a sample from a subject, wherein the systems or methods can further comprise measuring at least one additional parameter, such as PSA level, Gleason score and clinical stage. The invention is directed to systems and methods of predicting the probability of a recurrence of a cancer in a subject, wherein the systems or methods can further comprise measuring at least one additional parameter, such as PSA level, Gleason score and clinical stage. The invention further comprises prescribing and/or administering an appropriate therapy to a subject based on the predicted probability of recurrence.

### CROSS-REFERENCE TO RELATED APPLICATIONS

This application is a continuation-in-part of International Application No. PCT/US2013/054415, filed Aug. 9, 2013, which designated the U.S. and that International Application was published under PCT Article 21(2) in English. This application also includes a claim of priority under 35 U.S.C. §119(e) to U.S. provisional patent application No. 61/682,155, filed Aug. 10, 2012 and U.S. provisional patent application No. 62/021,648, filed Jul. 7, 2014, the entirety of which is hereby incorporated by reference.

### FIELD OF INVENTION

This invention relates to the fields of urology, oncology and pathology. More specifically, this invention relates to systems and methods for predicting the probability of prostate cancer recurrence in a subject before, during, or after cancer treatment. This invention also relates to systems and methods for detecting a cysteine level in a sample from a subject.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Prostate cancer remains the most common non-cutaneous solid malignancy in the United States, and the second leading cause of cancer specific death in men. Nevertheless, it has become increasingly clear that not all men who are diagnosed with prostate cancer require intervention [1]. Yet, many men that receive surgical or radiation-based primary treatment develop recurrent disease. Prior to surgical intervention, serum PSA, biopsy Gleason grade, and clinical stage help determine if patients are likely to be recurrent versus those that may remain localized and possibly remain clinically inconsequential. Various approaches in improving the role of PSA in early prostate cancer detection have been tested, but their benefit to overall survival is yet to be proven [2,3]. Ultimately, there is a subgroup of men without conventional negative factors harboring high risk, aggressive disease and are even at elevated risk of early recurrence after attempted definitive local therapy [4,5,6]. The ongoing challenge facing clinicians is how to identify this cohort of men at high risk, from the larger cohort of men who are likely harboring more indolent disease [7]. New markers of aggressive disease are therefore needed for an informed clinical decision.

A previous study identified sarcosine (N-methylglycine) as a product of methionine catabolism that is elevated in the urine of patients with metastatic prostate disease [8]. Sarcosine levels were higher in tissues from localized prostate cancer than in normal tissue, and even higher in metastatic prostate tissue. Urinary sarcosine was thus suggested as a possible marker for metastatic prostate cancer. The enzyme, Glycine N-methyltransferase (GNMT) is the primary source of sarcosine in liver, where it accounts for about 1% of the soluble protein [9]. Individuals with defective sarcosine dehydrogenase have sarcosinemia, but show no distinctive phenotype [10]. However, a reported causative role for sarcosine in prostate cancer metastasis [8], suggests therapeutic targeting of its metabolic pathway to be useful.

Nevertheless, the current markers only suggest the presence or absence of cancer and they are not shown to have any predicative value. As such, there still exists a great need for markers, methods and systems that can predict the probability of recurrent cancer. In this invention, we demonstrate that the cysteine level in urine or serum is a predictive marker for cancer recurrence. We provide systems and methods of predicting the probability of cancer recurrence based on the cysteine level, and we provide systems and methods of detecting the cysteine level in urine or serum using a combination of enzymes and nanorods.

### SUMMARY OF THE INVENTION

The following embodiments and aspects thereof are described and illustrated in conjunction with systems, compositions and methods which are meant to be exemplary and illustrative, not limiting in scope.

Various embodiments of the present invention provide for a system for detecting a cysteine level in a sample from a subject. The system can comprise cystathionine synthase, cystathionine lyase, and nanorods. The system can further comprise a PSA test, clinical stage, biopsy Gleason score, pathologic Gleason score, pathologic stage, surgical margin status, lymph node involvement, or seminal vesicle involvement, or a combination thereof.

Various embodiments of the present invention provide for a method of detecting a cysteine level in a sample from a subject. The method comprises: obtaining a sample from the subject; processing the sample with cystathionine synthase and cystathionine lyase; mixing the processed sample with nanorods; measuring a change of absorption spectrum of the nanorods; and detecting the cysteine level based upon the change of absorption spectrum.

Various embodiments of the present invention provide for a system for predicting the probability of a recurrence of a cancer in a subject. The system comprises an isolated sample from the subject, cystathionine synthase, cystathionine lyase, and nanrods. The system can further comprise a PSA test, clinical stage, biopsy Gleason score, pathologic Gleason score, pathologic stage, surgical margin status, lymph node involvement, or seminal vesicle involvement, or a combination thereof.

Various embodiments of the present invention provide for a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; and predicting an increased probability of the recurrence of the cancer in the subject when the cysteine level in the subject is detected to be higher than in non-recurrent subjects. The method can further comprise active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

Various embodiments of the present invention provide for a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; and predicting an increased probability of the recurrence of the cancer in the subject when the cysteine level in the subject is detected to be higher than in non-recurrent subjects and/or when the additional parameter in the subject is detected to be higher or lower than in non-recurrent subjects. The method can further comprise active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

Various embodiments of the present invention provide for a method of predicting the probability of a recurrence of a cancer in a subject. The method comprises: obtaining a sample from the subject; processing the sample with cystathionine synthase and cystathionine lyase; subjecting the processed sample to an assay to detect cysteine level; and predicting an increased probability of the recurrence of the cancer in the subject when the cysteine level in the subject is detected to be higher than in non-recurrent subjects. The method can further comprise active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

Various embodiments of the present invention provide for a system that comprises cystathionine synthase, cystathionine lyase, and a nanorod. The system can further comprise Cu²⁺. The system can further comprise an isolated sample from a subject. The system can further comprise a PSA test, clinical stage, biopsy Gleason score, pathologic Gleason score, pathologic stage, surgical margin status, lymph node involvement, or seminal vesicle involvement, or a combination thereof.

Various embodiments of the present invention provide for a method of detecting a cysteine level in a sample from a subject. The method comprises: obtaining a sample from the subject; processing the sample with cystathionine synthase and cystathionine lyase; contacting the processed sample with a nanorod; measuring a change of absorption spectrum of the sample; and detecting the cysteine level based upon the measured change of absorption spectrum.

Various embodiments of the present invention provide for a nanoelectronic device. The nanoelectronic device comprises: a first electrode with a first surface; a second electrode with a second surface; a hinge connecting the two electrodes, wherein the hinge is non-conductive; and an ammeter measuring the electric current flowing between the two electrodes, wherein the two electrodes have different electric potentials; wherein the first surface is functionalized to bind cysteine, wherein the second surface is not functionalized to bind cysteine, and wherein the two surfaces face each other.

Various embodiments of the present invention provide for a system that comprises: a nanoelectronic device, cystathionine synthase, cystathionine lyase, and a linker, wherein the linker has at least one free thiol group, wherein the linker has sufficient length to connect the two surfaces, and wherein the linker is conductive.

Various embodiments of the present invention provide for a method of detecting a cysteine level in a sample from a subject. The method comprises: obtaining a sample from the subject; processing the sample with cystathionine synthase and cystathionine lyase; contacting the processed sample to a nanoelectronic device; removing the processed sample; contacting a linker with the nanoelectronic device; measuring the electric current in the nanoelectronic device; and detecting the cysteine level based upon the measured electric current, wherein the measured electric current is directly or inversely proportional to the cysteine level.

Various embodiments of the present invention provide for a method that comprises: obtaining a sample from a subject; processing the sample with cystathionine synthase and cystathionine lyase; and detecting a cysteine level in the processed sample using an assay to determine cysteine level. The method can further comprise predicting an increased probability of a recurrence of a cancer in the subject when the detected cysteine level in the subject is higher than a reference cysteine level. The method can further comprise: assessing at least one additional parameter; and predicting an increased probability of a recurrence of a cancer in the subject when the detected cysteine level in the subject is higher than a reference cysteine level and when the additional parameter in the subject is detected to be higher or lower than in non-recurrent subjects. The method can further comprise prescribing a first therapy to the subject, when the detected cysteine level in the subject is not higher than a reference cysteine level, or prescribing a second therapy or both the first therapy and the second therapy, when the detected cysteine level in the subject is higher than a reference cysteine level.

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1D depict Kaplan-Meier plots indicating univariate predictive values of the recurrence-free survival based on pre-surgical serum in accordance with various embodiments of the present invention. The patients were separated into two groups, divided at median tissue level for (A) PSA, (B) homocysteine, (C) cystathionine, and (D) cysteine as significantly associated with time to recurrence (Table 5). Those subjects above the median expression level were termed upper half, whereas those below the median were termed lower half. The recurrence-free survival probabilities were estimated by the Kaplan-Meier method and the differences were tested using the log-rank test. Each of the dichotomous serum markers supported statistically significant differences in biochemical recurrence-free survival.

FIGS. 2A, 2B, and 2C depict Receiver Operator Curve (ROC) for a statistical model that can be used to predict recurrence of prostate cancer based on serum derived variables in accordance with various embodiments of the present invention. Serum PSA is compared to the added value of serum (FIG. 2A) homocysteine, (FIG. 2B) cystathionine, and (FIG. 2C) cysteine. In the ROC curve the probability with greater Area Under the Curve (AUC) support increased specificity and sensitivity over random guess, represented by the dotted diagonal line.

FIG. 3A depicts methionine metabolism. Methionine is first converted to SAM, the donor of methyl groups in all but one methyltransferase reaction. SAM may transfer the methyl group to a variety of compounds, X, by a group of specific enzymes to yield the methylated compounds, CH3-X (eg. methylated lipids, DNA, or proteins). Alternatively, SAM may transfer the methyl group to glycine to form sarcosine via the enzyme glycine N-methyltransferase (GNMT. After transfer of the methyl group SAM is converted to S-adenosylhomocysteine (SAH), which is broken down further to homocysteine, cystathionine and cysteine. Sarcosine may also be formed by breakdown of choline to betaine, which, after loss of a methyl group, is converted to dimethylglycine. A dehydrogenase converts dimethylglycine to sarcosine.

FIG. 3B depicts methionine metabolism. Methionine is first converted to SAM, the donor of methyl groups in all but one methyltransferase reaction. SAM may transfer the methyl group to a variety of compounds, X, by a group of specific enzymes to yield the methylated compounds, CH3-X (eg. methylated lipids, DNA, or proteins). Alternatively, SAM may transfer the methyl group to glycine to form sarcosine via the enzyme glycine N-methyltransferase (GNMT). After transfer of the methyl group SAM is converted to S adenosylhomocysteine (SAH), which is broken down further to homocysteine, cystathionine and cysteine. Sarcosine may also be formed by breakdown of choline to betaine, which, after loss of a methyl group, is converted to dimethylglycine. A dehydrogenase converts dimethylglycine to sarcosine. The biosynthesis of cysteine (the detected analyte) is a product of cystathionine beta-synthase (CBS) activity on homocysteine and further cystathionine gamma-lyase (CGL) activity in the hydrolysis of cystatothionine. CBS and CGL activity is exploited in the strategy of collapsing the cysteine metabolism pathway, enriching for the three highly predictive biomarkers for recurrent prostate cancer: homocysteine, cystathionine, and cysteine.

FIGS. 4A and 4B depict analysis of cysteine in serum with gold nanorods in accordance with various embodiments of the present invention. (A) Spectrophotometric scanning of the visible and infrared spectrum shows a distinctive red-shift in the absorbance when gold (Au) nanorods alone (dotted line) are subjected to human serum containing cysteine for 1, 4, and 6 minutes at room temperature. The arrow indicates the 950 nm wavelength at which the cysteine concentration is measured. (B) At concentration ranges of homocysteine, cystathionine, and cysteine, (analogous to that found in non-recurrent and recurrent subjects), Au nanorods were used to quantitate at 950 nm wavelength (black line). When cysteine enrichment was done prior to identical spectrophotometric detection of the same serum samples (grey line), the greater slope indicates greater sensitivity.

FIGS. 4C and 4D depict spectrophotometric scanning of the visible and infrared spectrum of cetyltrimethylammonium bromide (CTAB) protected naked gold nanorods (AuRd) in the presence and absence of CuCl₂ (Cu) in accordance with various embodiments of the present invention. (C) the boxed area indicates cysteine concentration-dependent gold nanoparticles aggregation in presence of HCl and CuCl₂. The infrared spectrum of interest is expanded to highlight the change in extinction at the 965 nm wavelength within the concentration range of 100 nM to 750 nM cysteine. (D) Shown is an extrapolation of the absorbance measurements to illustrate a standard curve with CTAB protected AuRd.

FIG. 5 depicts the strategy of using covalently protected gold nanorods to limit cysteine binding to the longitudinal aspect of the rods in accordance with various embodiments of the present invention. This limits random aggregation and enables assembly of longer coordinated structures. Cu²⁺ forms coordinate bonds with cysteine. The protection material can be metallic (eg. palladium, selenium, platinum), water-soluble polymer, or carbon.

FIGS. 6A-6B depict spectrophotometric scanning of visible and infrared spectrum of polymer protected gold nanorods in a time course in accordance with various embodiments of the present invention. A distinctive red-shift from baseline in the presence of CuCl₂ (Cu) and cysteine is observed. The dashed line (0 Cys, 0 min) in panel A indicates baseline absorbance of the nanorods with overlapping measurements with the solid line of 250 μM cysteine in the absence of Cu. The remaining dashed-lines at indicated times of incubation have a drift in the presence of Cu alone in a time dependent manner. The solid lines with indicated incubation times have an absence of any absorbance drift in the presence of Cu and 250 μM cysteine from 1 to 30 minute incubation time. In panel B there is a cysteine concentration-dependent red shift in the presence of Cu and constant 5 minute incubation time.

FIG. 7 depicts cysteine detection using polymer coated gold nanorods in a concentration range of 0 to 100 μM in the absence and presence CuCl₂ (Cu) in accordance with various embodiments of the present invention. The baseline absorbance is determined by the wavelength value in absence of Cu. The concentration-dependent red-shift is independent of incubation time 1 and 5 minutes. Further extended incubation of up to 30 minutes had no change in absorbance wavelength (data not shown).

FIG. 8 depicts cysteine detection using polymer coated gold nanorods (pRd) in accordance with various embodiments of the present invention. In panel A, a standard curve was generated by measuring cysteine in the concentration range of 1 to 100 μM. The cysteine-depended red shift is plotted to demonstrate saturation when testing detection 1 to 1000 μM cysteine (lower panel). The upper panel demonstrates the linearity of the red-shift (nm) with a R²=0.9508. Panel B demonstrates that mixtures of pRd and CTAB protected gold nanorods (cRd) can provide cysteine detection similar to pRd alone through a saturation curve (lower panel) and linear detection range (upper panel) as pRd alone.

FIG. 9 depicts analysis of cysteine in serum with polymer coated gold nanorods in accordance with various embodiments of the present invention. The inset illustrates a standard curve within a cysteine concentration of 0 to 100 μM cysteine. The bar graph extrapolates from the standard curve the change in peak position (left axis) to cysteine concentration (right axis). In this example, as the human serum sample was diluted ten-fold before the assay, the actual cysteine concentration in the human sample is 403.7 μM. The addition of exogenous cysteine (5 to 100 μM) to the cysteine had a linear red-shift. The addition of 100 μM cysteine to the serum (total of ˜140 μM cysteine) had saturated the polymer coated gold nanorods.

FIG. 10 depicts the detection of cystathionine and homocysteine by polymer-coated gold nanorods based on the thiol-dependent red shift observed with the detection of cysteine in accordance with various embodiments of the present invention. Panel A indicates a lack of cystathionine detection (solid line) compared to cysteine (dashed line) in a dose-dependent manner. Panel B indicates reduced homocysteine detection (solid line) compared to cysteine (dashed line) in a dose-dependent manner.

FIG. 11 depicts that the treatment of cystathionine and homocysteine with optimized cystathionine beta-synthase (oCBS) and gamma-lyase (oCGL) enables improvement of their detection with polymer coated gold nanorods in accordance with various embodiments of the present invention. Panel A is an acrylamide gel of purified recombinant optimized oCBS and oCGL expressed in E. coli. Panel B illustrates the efficacy of the enzymatic conversion of homocysteine and cystatothyonine for cysteine detection. The data demonstrates cysteine detection in samples using polymer coated gold nanorods in samples containing homocysteine and cytathionine in the presence and absence of oCBS and oCGL. Spectrophotometric scanning of the visible and infrared spectrum shows little shift with the addition of homocysteine and cystathionine, compared to baseline. A distinctive red-shift in absorbance was observed when oCBS and oCGL were incubated with homocysteine and cystathionine, as with the addition of cysteine. Panel C demonstrates the detection of cystathionine (100 μM) and homocysteine (100 μM) in the presence of oCBS and oCGL, compared to that without enzymatic treatment and cysteine alone (positive control).

FIG. 12A depicts nanoelectrode-based detection of cysteine in accordance with various embodiments of the present invention. In a nanoelectronic device, one of the two electrodes is a bare gold nanoelectrode capable of binding to cysteine or free thiol group (—SH) and the other electrode cannot bind to cysteine or free thiol group (—SH). Cysteine in a sample binds to the bare gold nanoelectrode. Then the two electrodes are connected by a linker, which is a conductive element allowing an electric current to pass between the two electrodes. The linker is nanoparticles (e.g., nanorods, nanospheres, nanofibers, nanowires, nanotubes) functionalized to have free thiol group (—SH) for binding to the remaining unoccupied binding sites on the bare gold nanoelectrode. The detected current will be inversely proportional to cysteine in the sample.

FIG. 12B depicts nanoelectrode-based detection of cysteine in accordance with various embodiments of the present invention. In a nanoelectronic device, one of the two electrodes is a bare gold nanoelectrode capable of binding to cysteine or free thiol group (—SH) and the other electrode cannot bind to cysteine or free thiol group (—SH). Cysteine in a sample binds to the bare gold nanoelectrode. Then the two electrodes are connected by a linker, which is a conductive element allowing an electric current to pass between the two electrodes. The linker is a flexible molecule having free thiol group (—SH) for binding to the remaining unoccupied binding sites on the bare gold nanoelectrode. The detected current will be inversely proportional to cysteine in the sample.

FIG. 12C depicts nanoelectrode-based detection of cysteine in accordance with various embodiments of the present invention. In a nanoelectronic device, one of the two electrodes is a bare gold nanoelectrode capable of binding to cysteine or free thiol group (—SH) and the other electrode cannot bind to cysteine or free thiol group (—SH). Cysteine in a sample binds to the bare gold nanoelectrode. Then the two electrodes are connected by a linker, which is a conductive element allowing an electric current to pass between the two electrodes. The linker is cysteine-bound nanoparticles. Cu²⁺ forms coordinate bonds with cysteine bound on the electrode and cysteine bound on the nanoparticles. As a result, the two electrodes are connected. The detected current will be directly proportional to cysteine in the sample.

FIG. 13A depicts nanoelectrode-based detection of cysteine in accordance with various embodiments of the present invention. In a nanoelectronic device, one of the two electrodes is functionalized to have free thiol group (—SH) for binding to cysteine or another free thiol group (—SH), and the other electrode cannot bind to cysteine or free thiol group (—SH). Cysteine in a sample form disulphide bond (—S—S—) with the free thiol group on the functionalized electrode. Then the two electrodes are connected by a linker, which is a conductive element allowing an electric current to pass between the two electrodes. The linker is nanoparticles (e.g., nanorods, nanospheres, nanofibers, nanowires, nanotubes) functionalized to have free thiol group (—SH) for binding to the remaining unoccupied binding sites on the functionalized nanoelectrode. The detected current will be inversely proportional to cysteine in the sample.

FIG. 13B depicts nanoelectrode-based detection of cysteine in accordance with various embodiments of the present invention. In a nanoelectronic device, one of the two electrodes is functionalized to have free thiol group (—SH) for binding to cysteine or another free thiol group (—SH), and the other electrode cannot bind to cysteine or free thiol group (—SH). Cysteine in a sample form disulphide bond (—S—S—) with the free thiol group on the functionalized electrode. Then the two electrodes are connected by a linker, which is a conductive element allowing an electric current to pass between the two electrodes. The linker is a flexible molecule having free thiol group (—SH) for binding to the remaining unoccupied binding sites on the functionalized nanoelectrode. The detected current will be inversely proportional to cysteine in the sample.

FIG. 14 depicts the types of nanoelectrodes and the types of linkers in accordance with various embodiments of the present invention. One of the two electrodes in the nanoelectronic device is capable of binding to cysteine or a free thiol (—SH). This electrode can be (A) bare gold nanoplates; (B) gold nanoplates functionalized with free thiol groups (—SH); and (C) other metallic nanoplates (e.g., selenium, cadmium, copper, platinum, palladium) or nonmetallic nanoplates (carbon, grapheme, or fullerene) functionalized with free thiol groups (—SH). The linker can be (D) nanoparticles functionalized with free thiol groups (—SH); (E) cysteine-bound nanoparticles with help of Cu²⁺ for binding to cysteine bound on the electrode; (F) a flexible molecule with free thiol groups (—SH). pH or ionic strength changes break up hydrogen or ionic bonds in the flexible molecule thereby opening up the flexible molecule. If its length is sufficient to cover the distance between the two electrodes, the flexible molecule itself can serve as the linker; otherwise, it can be optionally conjugated to a nanoparticle to form a “flexible molecule-nanoparticle” complex as a bigger linker molecule.

FIGS. 15A-15B depict, in accordance with various embodiments of the present invention, cloning, expression and purification of CBS and CGL.

FIGS. 16A-16F depict, in accordance with various embodiments of the present invention, CBS and CGL activity determination by HPLC.

FIGS. 17A-17D depict, in accordance with various embodiments of the present invention, cysteine titration using naked and CTAB protected gold nanorods.

FIGS. 18A-18G depict, in accordance with various embodiments of the present invention, pRd reaction with cysteine results in formation of linearly joined long chain nanopolymer.

FIGS. 19A-19B depict, in accordance with various embodiments of the present invention, change of plasmonic properties of pRd upon reaction with cysteine.

FIGS. 20A-20B depict, in accordance with various embodiments of the present invention, effects of acid and Cu2+ on cysteine induced reassembly of cRd.

FIGS. 21A-21B depict, in accordance with various embodiments of the present invention, pRd based sulfur amino acids titration standard curve.

FIG. 22 depicts, in accordance with various embodiments of the present invention, determination of CBS and CGL activity by plasmon shift assay.

FIGS. 23A-23C depict, in accordance with various embodiments of the present invention, serum cysteine concentration and its prognostic value in mice.

FIG. 24 depicts, in accordance with various embodiments of the present invention, detection of serum cysteine level in prostate cancer patients using pRd before and after enzymatic conversion of the biomarkers.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application. For references on how to prepare these antibodies, see D. Lane, Antibodies: A Laboratory Manual (Cold Spring Harbor Press, Cold Spring Harbor N.Y., 1988); Kohler and Milstein, (1976) Eur. J. Immunol. 6: 511; Queen et al. U.S. Pat. No. 5,585,089; and Riechmann et al., Nature 332: 323 (1988).

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. In some embodiments, the disease condition is cancer.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented. Examples of cancer treatment include, but are not limited to, active surveillance, surgical intervention, prostatectomy, chemotherapy, immunotherapy, hormone therapy, radiation therapy, focal therapy, systemic therapy, high frequency ultrasound (HIFU), cryo therapy, brachytherapy, or a combination thereof.

“Tumor,” as used herein refers to all neoplastic cell growth and proliferation, whether malignant or benign, and all pre-cancerous and cancerous cells and tissues.

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Chemotherapy resistance” as used herein refers to partial or complete resistance to chemotherapy drugs. For example, a subject does not respond or only partially responds to a chemotherapy drug. A person of skill in the art can determine whether a subject is exhibiting resistance to chemotherapy.

“Cystathionine synthase” is an enzyme that catalyzes the reaction of from homocysteine to cystathionine. In various embodiments, the cystathionine synthase is cystathionine beta-synthase. Examples of “cystathionine synthase” include but are not limited to polypeptides comprising a sequence as set forth in SEQ ID NO: 1 or SEQ ID NO: 5. Also in accordance with various embodiments of the present invention, the cystathionine synthase can comprise a variant or mutant of the sequence as set forth in SEQ ID NO: 1 or SEQ ID NO: 5.

“Cystathionine lyase” is an enzyme that catalyzes the reaction of from cystathionine to cysteine. In various embodiments, the cystathionine lyase is cystathionine gamma-lyase. Examples of “cystathionine lyase” include but are not limited to polypeptides comprising a sequence as set forth in SEQ ID NO: 8 or SEQ ID NO: 12. Also in accordance with various embodiments of the present invention, the cystathionine lyase can comprise a variant or mutant of the sequence as set forth in SEQ ID NO: 8 or SEQ ID NO: 12.

The “a variant” or “a mutant” as used herein includes, but is not limited to, a nucleic acid or polypeptide with a mutation, a deletion, an insertion, or a fusion, or a combination thereof, as compared to a wild type or reference sequence.

A “nanoparticle” is a particle having one or more dimensions of the order of 100 nm or less. A nanoparticle can be made of a variety of materials, including but limited to, gold, selenium, cadmium, copper, platinum, palladium, or carbon, or a combination thereof. A nanoparticle can take a variety of shapes, including but limited to, rod, sphere, fiber, wire, or tube, or a combination thereof. As examples, nanofibers are fibers with diameters less than 100 nanometers; nanowires are about 75 nm in diameter, and range from 1 μm to 10 microns in length; nanotube are cylindrical nanoscale structures with length-to-diameter aspect ratios of up to 132,000:1. These particles can be bare, or can be capped with carboxylic acid, conventional citrate, and/or a positively charged ligand. These capping agents can readily be replaced with covalent and charge chemistries.

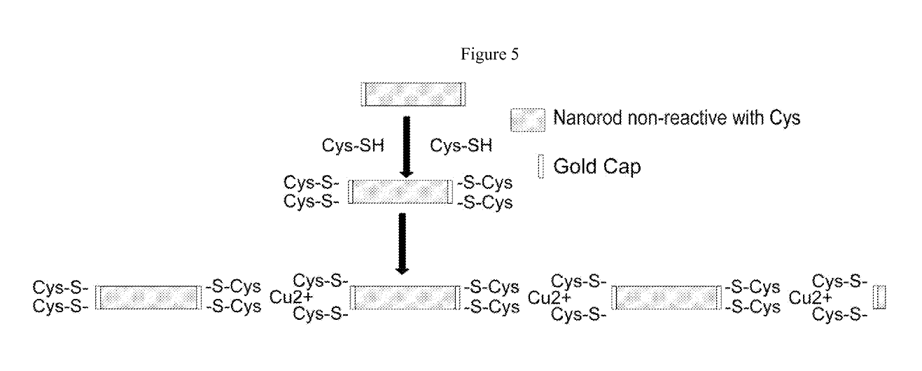

Nanorods are one morphology of nanoscale objects. Their dimensions usually range 1-100 nm, and their aspect ratios (length divided by width) usually range 3-5. Nanorods may be synthesized from metals or semiconducting materials or their combinations. A nanorod has two ends and a linear body between the two ends. The two ends are also called the transverse or shorter ends. Accordingly, the longitudinal surface of the linear body is also called the longitudinal or longer end. The cross section of the linear body can be shaped as a variety of shapes, examples of which include but are not limited to, sphere, rectangular prism, dumbbell, triangle, rectangle, hexagon, or octagon, or a combination thereof. The two ends and the linear body may be made of the same or different materials. For example, a nanorod can be made by capping the two ends of a carbon or an inert metal linear body with two gold caps (FIG. 5). An “end surface” as used herein refers to the total area of an end plus the 0-10% of the linear body adjacent to the end; as a nanorod has two end surfaces, a “longitudinal surface” as used herein refers to the remaining 80-100% area of the linear body between the two end surfaces.

A “recurrence” means that the cancer has returned after initial treatment. For example, a recurrence of prostate cancer means that the prostate cancer has returned after initial treatment. When prostate cancer is caught in its earliest stages, initial therapy can lead to high chances for cure, with most men living cancer-free for at least five years. But prostate cancer can be slow to grow following initial therapy, and it has been estimated that about 20-30% of men will relapse after the five-year mark and begin to show signs of disease recurrence. A rising PSA is typically the first sign seen, coming well before any clinical signs or symptoms. Rise in serum PSA 0.2 ng/ml indicates biochemical recurrence. Rapidly recurrent patients were identified as those who developed biochemical recurrence following prostatectomy within 2 years (American Joint Committee on Cancer defined as having PSA 0.2 ng/ml, confirmed at least once two weeks later). The recurrence-free population was defined as having maintained a serum PSA<0.01 ng/ml for five or more years following surgery. Being non-recurrent or recurrence-free means that the cancer is in remission; being recurrent means that the cancer is growing and/or has metastasized, and some surgery, therapeutic intervention, and/or cancer treatment is required to prevent lethality. The “non-recurrent subjects” are subjects who have non-recurrent or recurrence-free disease, and they can be used as the control population in various embodiments of the present invention.

Prostate cancer remains the most common non-cutaneous solid malignancy in the United States, and the second leading cause of cancer specific death in men. Nevertheless, it has become increasingly clear that not all men who are diagnosed with prostate cancer require intervention. The continuing problem is that we do not know how to distinguish the estimated 80% of prostate cancer patients that may not need invasive therapy from those who need treatment at an early stage. This dilemma results in unnecessary health care cost, subjecting individuals to a major intervention (surgical or radiation) that has a clear negative impact to quality of life, and sometimes not acting soon enough for patients that need aggressive intervention.

Higher serum homocysteine, cystathionine, and cysteine concentrations independently predicted risk of early biochemical recurrence and aggressiveness of disease in a nested case control study. The methionine metabolites further supplemented known clinical variables to provide superior sensitivity and specificity in multivariable prediction models for rapid biochemical recurrence following prostatectomy. This could be especially useful for prostate cancer patients considering radiation as their primary treatment. In the current health care environment, aggressive treatments like prostatectomies and even radiation prostate ablative therapies are being reconsidered, due to cost and possible limitations in patient benefit. The biomarkers identified can potentially identify subjects who would require aggressive definitive treatment versus those who would be better served by active surveillance. Various embodiments of the present invention provide predictive biomarkers of cancer recurrence, as well as systems and methods of marker detection that can be both cost efficient and highly sensitive.

Various embodiments of the present invention provide a marker that predicts indolent disease versus recurrent and aggressive disease. Recent publications state that as many as 80% of the surgeries are unnecessary since there is a significant number of patients with indolent disease. The present invention helps us to prevent unnecessary major surgery. This is attractive as a means of saving healthcare dollars and preventing complications. For patients with recurrent disease, the current standard of care is to wait for recurrence, prior to adjuvant therapies. There is a consensus that it is too late at that point, since all adjuvant therapies are not curative. Salvage radiation immediately following prostatectomy has been proven to prevent recurrence. However, such salvage radiation is not practical for all patients, since pelvic floor radiation is associated with significant side effects and most patients may not need the aggressive therapy in the first place. Therefore, this test will help in making the decision on who needs the aggressive intervention.

Various embodiments of the present invention provide unique detection methods involving cysteine detection in patient urine and serum samples using gold nanorod technology in combination of enzymes. The gold nanorod technology has not been used in the clinical setting due to the lack of specificity for thiol group containing amino acids and their metabolites, including homocysteine, cystathionine, and cysteine. Simply, the three thiol containing metabolites are of different length, which result in a broadened and diminished absorption peak due to heterogeneous assembly of the nanorods. This is more of an issue for cytathionine, since the thiol group is in a different position from that of homocysteine and cysteine. A method of converting the methionine metabolism pathway components, thereby increasing both specificity and sensitivity in serum and urine has been developed and is described herein.

According to various embodiments of the present invention, medical practitioners can now use pre-surgical variables to determine if the patient is likely to have recurrent disease in order to act aggressively during surgery and immediately following surgery with adjuvant therapy, with the goal of preventing recurrence. Insurance companies and governments would appreciate that its use would save significant health care dollars in treatment and future care from complications from unnecessary surgical or radiation intervention. Urologists and Oncologists would be able to use the present invention to prescribe primary and adjuvant intervention at an earlier stage in cancer progression, following definitive care, prior to any other metastasis detection method.

Current risk stratification of patients prior to surgery involves variables including serum PSA, clinical stage, and biopsy grade. Independent serum markers in conjunction with PSA could help distinguish patients with aggressive prostate cancer. In the current era of PSA testing, clinical staging has reduced relevance when tumor volumes are relatively small. In our study, the highest biopsy Gleason score in ≧8-core biopsies provided a significant independent predictor comparable to serum cysteine and homocysteine. However, routine ultrasound directed first biopsies are reported to miss nearly a quarter of the prostate cancers [19] and often underestimate tumor grade [20,21]. The combination of serum PSA with cystathionine, cysteine, and homocysteine as markers could improve decision-making for primary treatment and earlier subsequent adjuvant therapy.

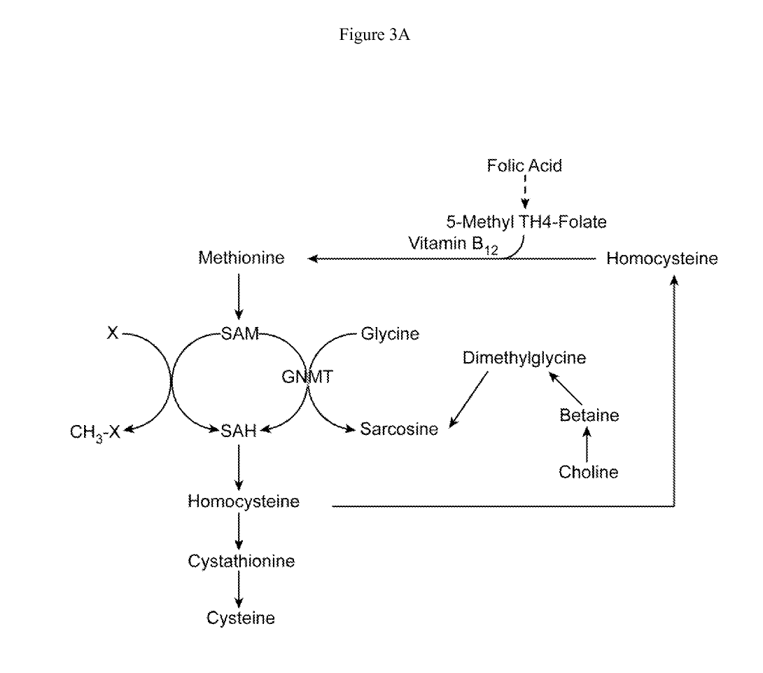

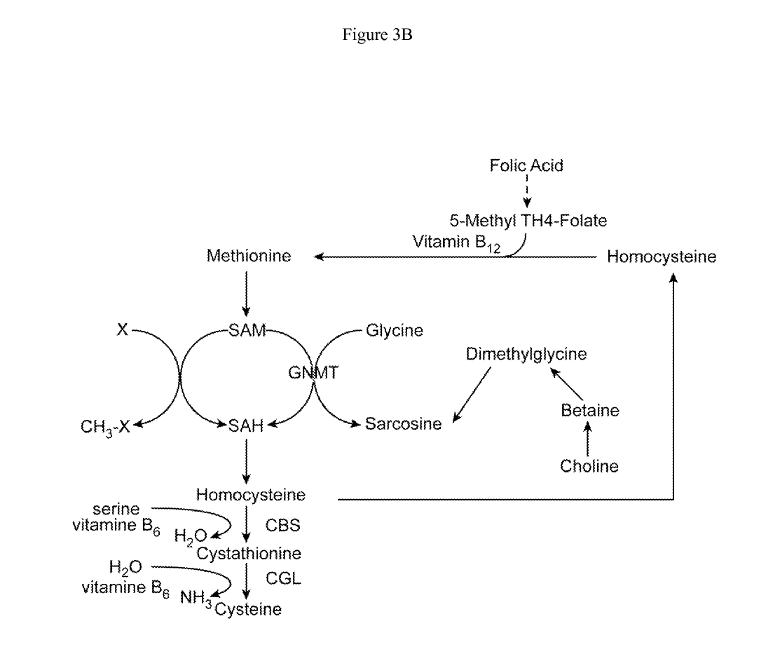

Pathways of methionine metabolism involve two mechanisms for sarcosine formation (FIG. 3). Cystathionine and cysteine are products of homocysteine catabolism important in production of glutathione. Elevation of urinary sarcosine in the absence of serum sarcosine differences was surprising, and likely the result of differential renal sarcosine excretion. Changes in sarcosine but not dimethylglycine suggest that increased activity of GNMT might have been present in the recurrent group. It is possible that for unknown reasons the recurrent group had increased S-adenosylmethionine (SAM) which activated the transulfuration pathway [22] thus, increasing cystathionine, cysteine, and formation of sarcosine. It should be noted that Sreekumar et al. [8] did not report sarcosine in patient serum or plasma associated with metastatic prostate cancer. Our data in pre-surgical subjects supports the previous report of urinary sarcosine elevation in confirmed metastatic patients. The data could mean that our patient population had previously undetected metastasis or that the elevated methionine metabolism is a precursor for metastasis. The direct role of sarcosine on metastatic progression is controversial. In contrast to the report of sarcosine directly supporting metastasis [8], a recent report suggests no association between urinary sarcosine levels and either tumor stage or Gleason score [23]. It is difficult to compare our findings with other reports since the initial study by Sreekumar et al [8] differ in the methodology of sarcosine measurement [24], sample source [25,26], and importantly criteria defining recurrence [23-27]. Our assay utilizes a stable isotope internal standard in each sample, retrieved urine and serum prior to prostate resection, and recurrence was only based on serum PSA detection. Another study compared benign controls against patients with active prostate cancer and found that urine sarcosine was only a modest predictor of disease, but when added to other new markers such as prostate cancer antigen 3 and percent-free PSA improved diagnostic power [27]. There is abundant evidence that folate and B12 deficiency and kidney disease can contribute to hyperhomocysteinemia. However, in the present investigation there was no difference in folate or methylmalonic acid levels between recurrent and non-recurrent groups. The patients in this study were accordingly recruited to minimize complicating co-morbidities. The differences we found in homocysteine, cystathionine and cysteine in serum suggest that there may be systemic metabolic differences in those patients who go on to have a biochemical relapse.

The majority of the sarcosine produced in the body is made in the liver as a downstream product of SAM and homocysteine. Studies using homozygous mice with GNMT knocked out have plasma SAM levels 50% greater than that of wild type. The SAM levels in the livers of the Gnmt null animals were 33 fold higher than in the livers of wild type animals and all of the Gnmt null animals developed hepatocellular carcinoma after 8 months [28]. Interestingly, higher cysteine values are associated with obesity [29-32]. The limited body composition data for our subject groups, however suggested little correlation of body mass index and recurrence rate. The data reported here, support increased flux through GNMT resulting in the increased formation of homocysteine and sarcosine through increased utilization of SAM. Interestingly, GNMT, is reported to be down-regulated in neoplastic tissues in general [33] including human prostate cancer [34]. Thus, the changes seen in the current investigation may not be a result of neoplastic changes in prostate tissue. While not wishing to be bound by any particular theory, we believe that these results suggest that there may be differences in the methylation capacity of different individuals or tumor hosts as a result of different levels of SAM. Unfortunately, SAM values could not be measured in the current study, because of the instability of SAM in stored serum samples. Further, it is possible that individuals with a greater methylating capacity are more likely to develop cancer leading to metastatic progression.

To our knowledge, no previous study has correlated an entire arm of a metabolic pathway in the aggressiveness of cancer. The comparison described here was made between patients with proven cancer, not between subjects with proven cancer and benign prostatic disease. The underlying biology supports the robustness of these markers. Higher serum homocysteine, cystathionine, and cysteine improved the utility of currently used clinical variables in predicting early recurrence and suggest a greater flux of methyl groups through the enzyme GNMT.

In various embodiments, the invention provides a system that comprises an isolated sample from a subject, cystathionine synthase, cystathionine lyase and nanorods. In accordance with various embodiments of the present invention, the system can be used to predict the probability of a recurrence of a cancer in a subject. In various embodiments, the subject can be human, monkey, ape, dog, cat, cow, horse, goat, pig, rabbit, mouse, or rat. In various embodiments, the sample can be serum, urine, blood, plasma, saliva, semen, lymph, or a combination thereof. In various embodiments, the cystathionine synthase comprises a polypeptide having a sequence as set forth in SEQ ID NO: 1. In various embodiments, the cystathionine lyase comprises a polypeptide having a sequence as set forth in SEQ ID NO: 8. In various embodiments, the nanorods can be made of gold, selenium, cadmium, copper, or a combination thereof.

In further embodiments, the invention provides a system that comprises an isolated sample from a subject, cystathionine synthase, cystathionine lyase, nanorods, and further comprises a PSA test, clinical stage, biopsy Gleason score, pathologic Gleason score, pathologic stage, surgical margin status, lymph node involvement, or seminal vesicle involvement, or a combination thereof. PSA level, clinical stage, and biopsy Gleason score have pre-surgical predictive value. Post-surgical standard of care information such as pathologic Gleason score, pathologic stage, surgical margin status, lymph node involvement, and seminal vesicle involvement can also augment the use of cysteine quantitation. In accordance with various embodiments of the present invention, the system can be used to predict the probability of a recurrence of a cancer in a subject. In some embodiments, the PSA test is a test of PSA velocity and/or total PSA level. PSA velocity means the rate at which PSA level rises over time.

In various embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; and predicting an increased probability of the recurrence of the cancer in the subject when the cysteine level in the subject is detected to be higher than in non-recurrent subjects. In various embodiments, the recurrence can be biochemical recurrence. In various embodiments, the cancer is prostate cancer, colon cancer, breast cancer, lung cancer, renal cancer, or bladder cancer. In various embodiments, the subject can be human, monkey, ape, dog, cat, cow, horse, goat, pig, rabbit, mouse, or rat. In various embodiments, the sample can be obtained before, during, or after cancer treatment. In various embodiments, the sample can be serum, urine, blood, plasma, saliva, semen, lymph, or a combination thereof. In some embodiments, the sample is urine and the urine cysteine level in the subject is above about 210 nanomoles of cysteine per milligram creatinine. In some embodiments, the sample is urine and the urine cysteine level in the subject is above about 220 nanomoles of cysteine per milligram creatinine. In some embodiments, the sample is urine and the urine cysteine level in the subject is above about 230 nanomoles of cysteine per milligram creatinine. In some embodiments, the sample is serum and the serum cysteine level in the subject is above about 400 μM of cysteine. In some embodiments, the sample is serum and the serum cysteine level in the subject is above about 410 μM of cysteine. In some embodiments, the sample is serum and the serum cysteine level in the subject is above about 420 μM of cysteine. In various embodiments, the nanorods can be made of gold, selenium, cadmium, copper, or a combination thereof.

In further embodiments, the invention provides a method of predicting the probability of a recurrence of a cancer in a subject and treating the subject. The method comprises: obtaining a sample from the subject; processing the sample with cystathionine synthase and cystathionine lyase; subjecting the processed sample to an assay to detect cysteine level, wherein the assay comprises nanorods; predicting an increased probability of the recurrence of the cancer in the subject when the cysteine level in the subject is detected to be higher than in non-recurrent subjects; and treating the subject with active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

In various embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; and predicting an increased probability of the recurrence of the cancer in the subject when the cysteine level in the subject is detected to be higher than in non-recurrent subjects and/or when the additional parameter in the subject is detected to be higher or lower than in non-recurrent subjects. In various embodiments, the recurrence can be biochemical recurrence. In various embodiments, the cancer can be prostate cancer, colon cancer, breast cancer, lung cancer, renal cancer, or bladder cancer. In various embodiments, the subject can be human, monkey, ape, dog, cat, cow, horse, goat, pig, rabbit, mouse, or rat. In various embodiments, the sample can be obtained before, during, or after cancer treatment. In various embodiments, the sample can be serum, urine, blood, plasma, saliva, semen, lymph, or a combination thereof. In some embodiments, the sample is urine and the urine cysteine level in the subject is above about 210 nanomoles of cysteine per milligram creatinine. In some embodiments, the sample is urine and the urine cysteine level in the subject is above about 220 nanomoles of cysteine per milligram creatinine. In some embodiments, the sample is urine and the urine cysteine level in the subject is above about 230 nanomoles of cysteine per milligram creatinine. In some embodiments, the sample is serum and the serum cysteine level in the subject is above about 400 μM of cysteine. In some embodiments, the sample is serum and the serum cysteine level in the subject is above about 410 μM of cysteine. In some embodiments, the sample is serum and the serum cysteine level in the subject is above about 420 μM of cysteine. In various embodiments, the nanorods can be made of gold, selenium, cadmium, copper, or a combination thereof.

In further embodiments, the invention provides a method of predicting the probability of a recurrence of a cancer in a subject and treating the subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; predicting an increased probability of the recurrence of the cancer in the subject when the cysteine level in the subject is detected to be higher than in non-recurrent subjects and/or when the additional parameter in the subject is detected to be higher or lower than in non-recurrent subjects; and treating the subject with active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

In further embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level, wherein the assay comprises nanorods; assessing at least one additional parameter; and predicting an increased probability of the recurrence of the cancer in the subject when the cysteine level in the subject is detected to be higher than in non-recurrent subjects and/or when the additional parameter in the subject is detected to be higher or lower than in non-recurrent subjects. In some embodiments, the additional parameter is PSA velocity, PSA level, pre-surgical PSA level, post-surgical PSA level, pre-treatment PSA level, post-treatment PSA level, biopsy Gleason score, clinical stage, number of positive cores, number of negative cores, Karnofsky performance status, Hemoglobin value, Lactate dehydrogenase value, Alkaline phosphatase value, Albumin level, urinary albumin level, or urinary creatinine level, or a combination thereof. Urinary albumin level and urinary creatinine level can also be used to assess if the subject has good liver and kidney functions. Urinary creatinine level can also be used to normalize differences in urine volume when measuring urinary cysteine levels. In some further embodiments, the additional parameter is a pre-treatment parameter comprising pre-treatment PSA level, pre-treatment biopsy Gleason Score, pre-treatment clinical stage, pre-treatment urinary albumin level, or pre-treatment urinary creatinine level, or a combination thereof. In some embodiments, the PSA level in the subject is above about 6.0 ng/ml in serum. In some embodiments, the Gleason score in the subject is above 7.

In various embodiments, the invention provides a method of predicting the probability of a recurrence of a cancer in a subject. The method comprises: obtaining a sample from the subject; processing the sample with cystathionine synthase and cystathionine lyase; subjecting the processed sample to an assay to detect cysteine level; and predicting an increased probability of the recurrence of the cancer in the subject when the cysteine level in the subject is detected to be higher than in non-recurrent subjects. In various embodiments, the recurrence can be biochemical recurrence. In various embodiments, the cancer can be prostate cancer, colon cancer, breast cancer, lung cancer, renal cancer, or bladder cancer. In various embodiments, the subject can be human, monkey, ape, dog, cat, cow, horse, goat, pig, rabbit, mouse, or rat. The sample can be obtained before, during, or after cancer treatment. In various embodiments, the sample can be serum, urine, blood, plasma, saliva, semen, lymph, or a combination thereof. In some embodiments, the sample is urine and the urine cysteine level in the subject is above about 210 nanomoles of cysteine per milligram creatinine. In some embodiments, the sample is urine and the urine cysteine level in the subject is above about 220 nanomoles of cysteine per milligram creatinine. In some embodiments, the sample is urine and the urine cysteine level in the subject is above about 230 nanomoles of cysteine per milligram creatinine. In some embodiments, the sample is serum and the serum cysteine level in the subject is above about 400 M of cysteine. In some embodiments, the sample is serum and the serum cysteine level in the subject is above about 410 μM of cysteine. In some embodiments, the sample is serum and the serum cysteine level in the subject is above about 420 μM of cysteine.

In further embodiments, the invention provides a method of predicting the probability of a recurrence of a cancer in a subject and treating the subject. The method comprises: obtaining a sample from the subject: processing the sample with cystathionine synthase and cystathionine lyase; subjecting the processed sample to an assay to detect cysteine level; predicting an increased probability of the recurrence of the cancer in the subject when the cysteine level in the subject is detected to be higher than in non-recurrent subjects; and treating the subject with active surveillance, prostatectomy, chemotherapy, immunotherapy, hormone therapy, radiation therapy, focal therapy, systemic therapy, high frequency ultrasound (HIFU), cryo therapy, brachytherapy, or a combination thereof.

In various embodiments, the invention provides a system that comprises cystathionine synthase, cystathionine lyase and nanorods. In accordance with various embodiments of the present invention, the system can be used to detect a cysteine level in a sample from a subject. In various embodiments, the subject can be human, monkey, ape, dog, cat, cow, horse, goat, pig, rabbit, mouse or rat. In various embodiments, the sample can be serum, urine, blood, plasma, saliva, semen, lymph, or a combination thereof. In various embodiments, the cystathionine synthase comprises a polypeptide having a sequence as set forth in SEQ ID NO: 1. In various embodiments, the cystathionine lyase comprises a polypeptide having a sequence as set forth in SEQ ID NO: 8. In various embodiments, the nanorods can be made of gold, selenium, cadmium, copper, or a combination thereof.

In further embodiments, the invention provides a system that comprises cystathionine synthase, cystathionine lyase, nanorods, and further comprise a PSA test, clinical stage, biopsy Gleason score, pathologic Gleason score, pathologic stage, surgical margin status, lymph node involvement, or seminal vesicle involvement, or a combination thereof. PSA level, clinical stage, and biopsy Gleason score have pre-surgical predictive value. Post-surgical standard of care information such as pathologic Gleason score, pathologic stage, surgical margin status, lymph node involvement, and seminal vesicle involvement can also augment the use of cysteine quantitation. In accordance with various embodiments of the invention, the system can be used to predict the probability of a recurrence of a cancer in a subject. In some embodiments, the PSA test is a test of PSA velocity and/or total PSA level. PSA velocity means the rate at which PSA level rises over time.

In various embodiments, the invention provides a method of detecting a cysteine level in a sample from a subject. The method comprises: obtaining a sample from the subject; processing the sample with cystathionine synthase and cystathionine lyase; mixing the processed sample with nanorods; measuring a change of absorption spectrum of the nanorods; and detecting the cysteine level based upon the change of absorption spectrum. In various embodiments, the sample can be serum, urine, blood, plasma, saliva, semen, lymph, or a combination thereof. In various embodiments, the subject can be human, monkey, ape, dog, cat, cow, horse, goat, pig, rabbit, mouse or rat. In various embodiments, the cystathionine synthase comprises a polypeptide having a sequence as set forth in SEQ ID NO: 1. In various embodiments, the cystathionine lyase comprises a polypeptide having a sequence as set forth in SEQ ID NO: 8. In various embodiments, the nanorods can be made of gold, selenium, cadmium, copper, or a combination thereof.

We evaluated the serum and urine of radical prostatectomy patients for metabolites to differentiate those who developed early biochemical recurrence (rise in serum PSA≧0.2 ng/ml) within two years of surgery and those who remained recurrence-free after more than five years. We found that the urine of patients in the rapidly recurrent group had significantly higher concentrations of sarcosine and cysteine than those in the recurrence-free group. In addition, significantly greater concentrations of serum cystathionine, homocysteine and cysteine were found in the rapidly recurrence group compared to the recurrence-free group. These products of elevated methionine catabolism in patients with rapidly recurrent prostate cancer represent pre-surgical indicators that augmented serum PSA for the prediction of clinically significant prostate cancer.

As shown in FIG. 3, cysteine is the last step of the methionine metabolism pathway. Cysteine is the most abundant in both urine and serum and is the most reflective of alterations in any component of the pathway that patients have. Cysteine is a superior serum or urine-based predictor of biochemical recurrence following prostatectomy than any previous report.

The current standard for cysteine detection involves gas chromatography and mass spectrometry. It involves the use of radio-labeled metabolites for the development of a standard curve and subsequent detection of the metabolites in the patient samples. This process is a highly complex, labor intensive and costly.

Here we developed a simple detection method of cysteine level. Our test has high sensitivity and specificity for predicting the probability of cancer recurrence. This finding has implications for patients with the highest chance of developing metastatic progression. If an urologist or oncologist knows a patient is more or less likely to have aggressive cancer the mode of intervention can be personalized. Prostate cancer patients uniquely benefit from such information since majority of patients harbor an indolent localized disease. Active surveillance can then be an informed option patients can make. Similarly, prostate cancer patients are normally not given adjuvant therapy until after frank recurrence detection. At this stage, all treatment options are non-curative. Early aggressive therapy is reported by multiple groups to have significant survival benefit for high risk patients.

Gold nanorods have not been used for detection of cysteine in serum in a clinical setting. The technology is based on the fact that thiol groups (—SH) found in cysteine bind to the gold and cause the nanorods to align linearly to result in a change in light absorption detected by a spectrophotometer. However, in a clinical setting where cysteine is not the only thiol containing molecule in the serum or urine, the nanorods cannot distinguish one from another. For example if homocysteine (also having a free thiol group available for gold rod interaction) is in the sample it could interfere with cysteine detection. Similarly cystathionine (also with a thiol group) could affect cysteine detection. There is a factor of diminished sensitivity when testing a heterogeneous sample. As a result, those patients who do not have over the top methionine metabolite levels would be mistakenly predicted with non-recurrent disease, when in fact they may have recurrent disease.

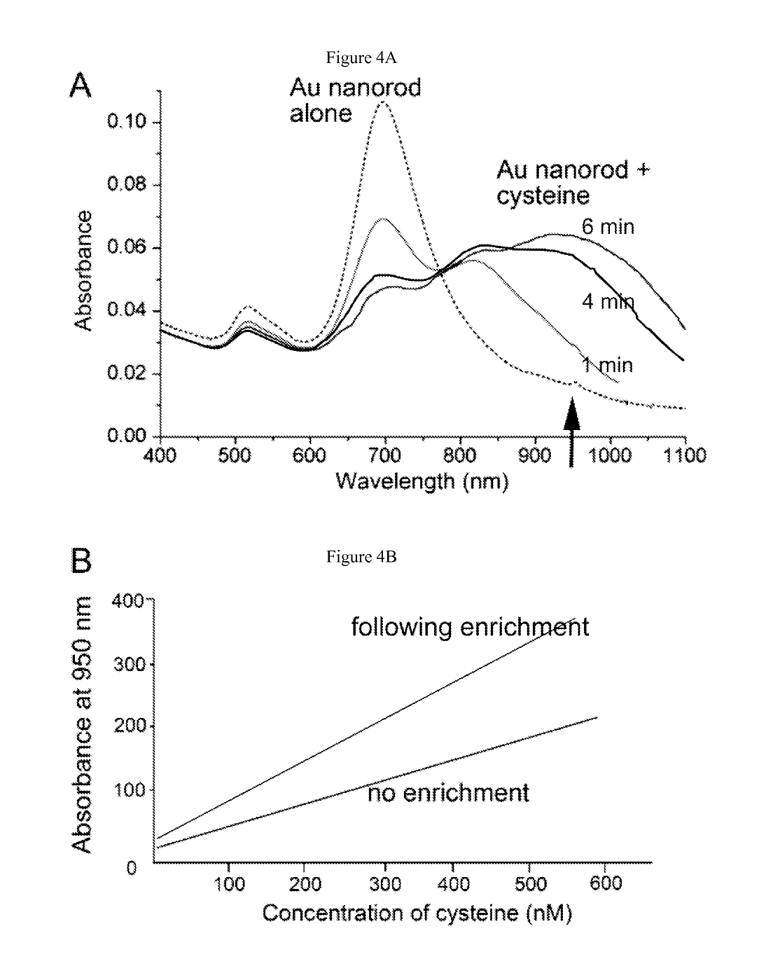

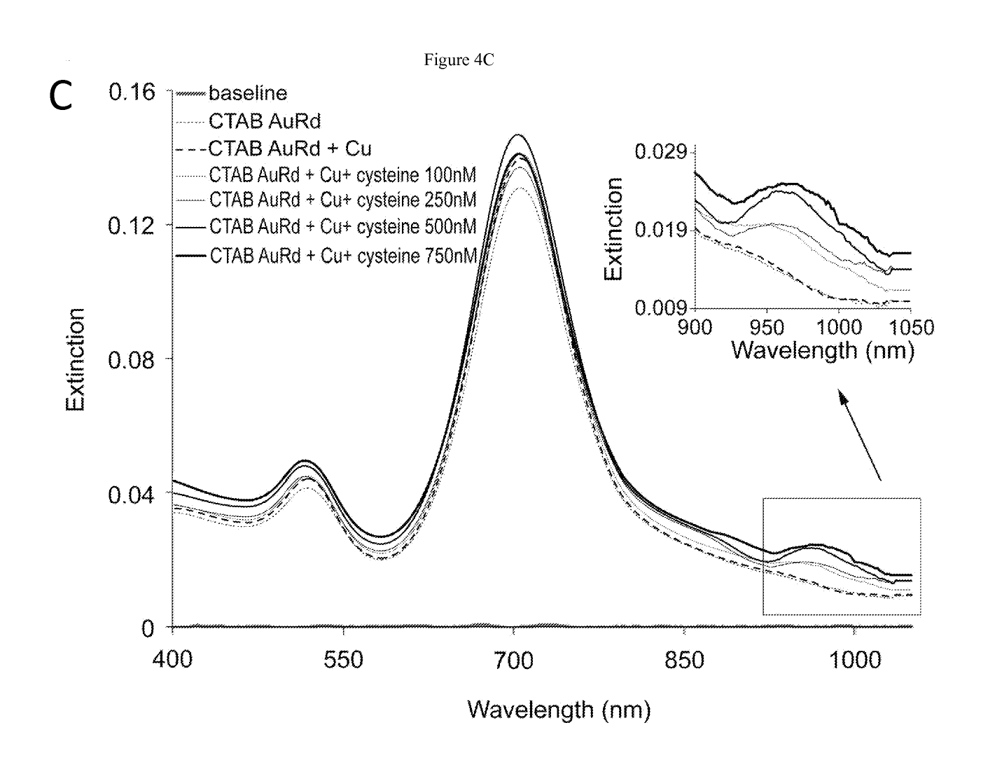

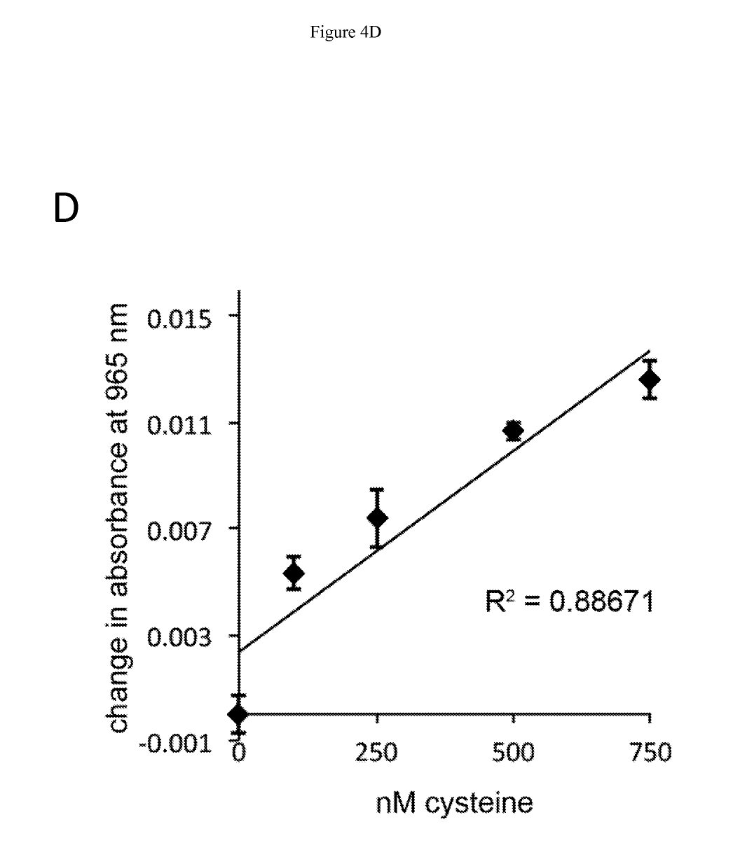

As shown in FIGS. 4A and 4B, this invention solves this problem both by converting both homocysteine and cystathionine to cysteine and detecting the final product, cysteine. This is highly effective since we showed that homocysteine, cystathionine, and cysteine independently have strong predictive value in multi variant cox analysis including standard clinical variables of biopsy Gleason grade, serum prostate specific antigen (PSA), and clinical stage.

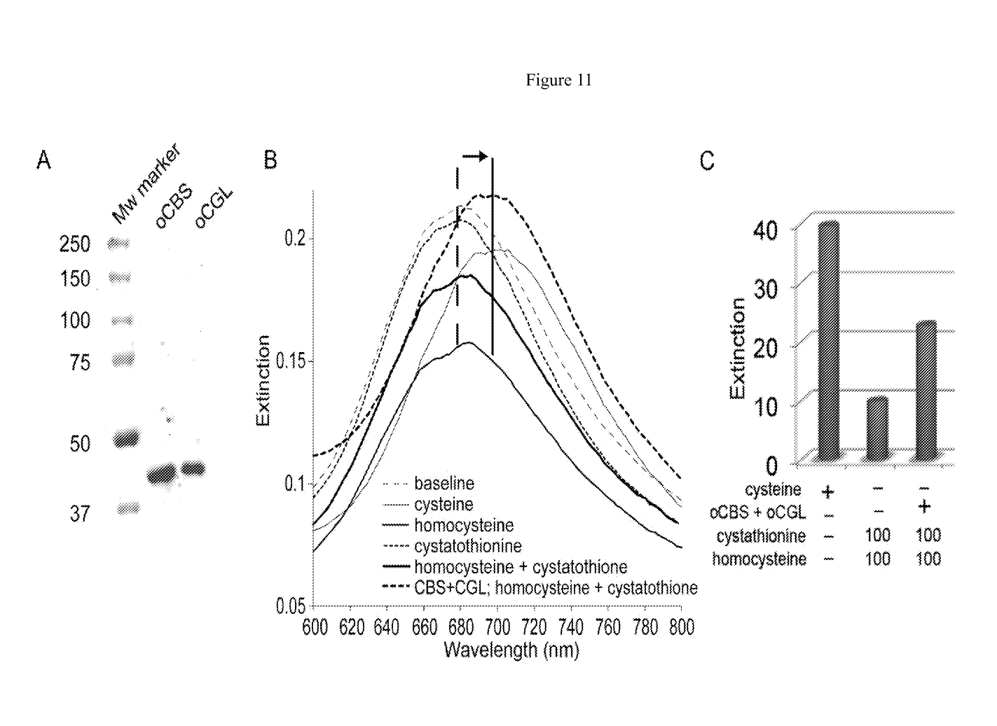

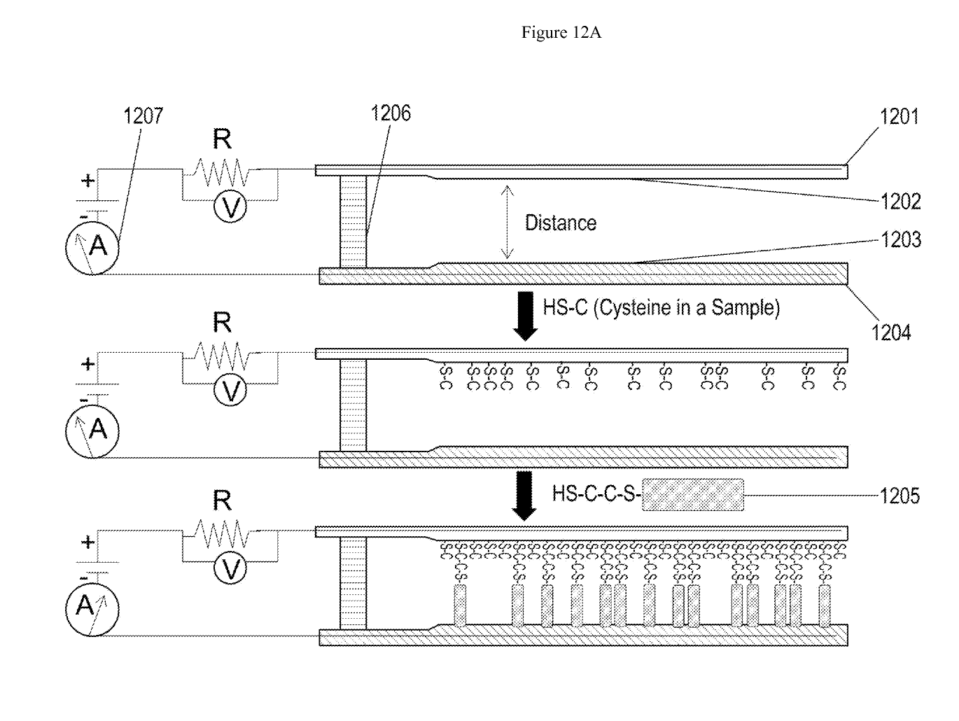

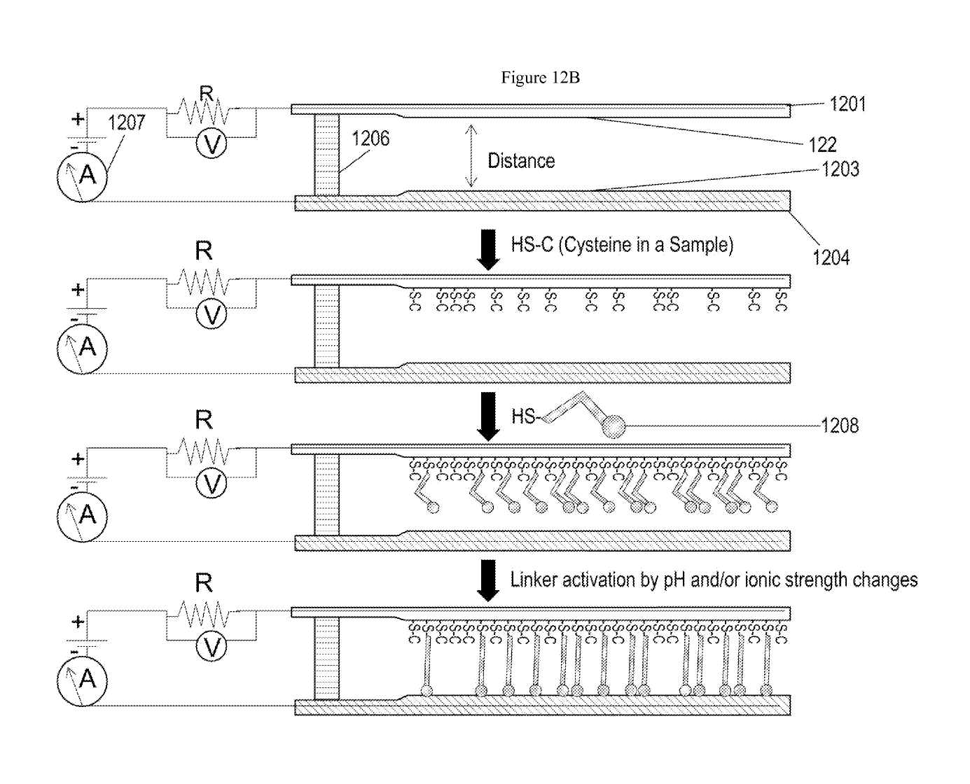

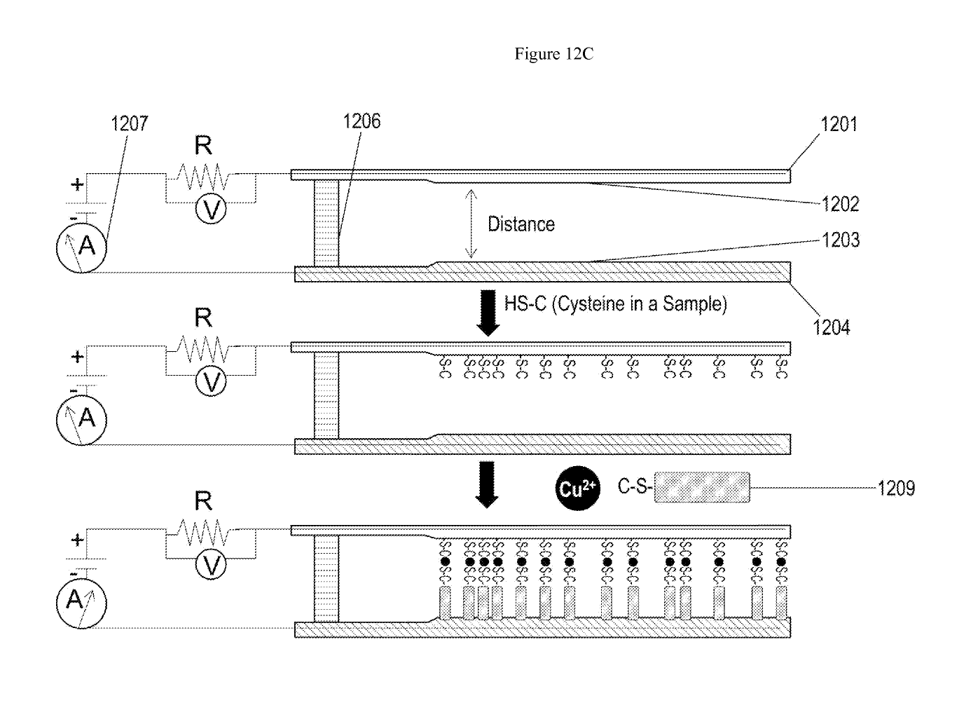

The present invention provides a method for preparing a sample for an assay to detect homocysteine, cystathionine, and cysteine level and a method of detecting homocysteine, cystathionine, and cysteine level in a sample from a subject. A typical analysis was realized by the following steps. A urine or serum sample is taken and processed with cystathionine synthase (e.g., cystathionine beta-synthase) and cystathionine lyase (e.g., cystathionine gamma-lyase) to convert homocysteine and cystathionine to cysteine enzymatically in vitro. As examples, cystathionine beta-synthase and cystathionine gamma-lyase are cloned from the Helicobacter pylori genome, and can be modified and optimized. The enzymatic reaction can be performed for about 10, 20, or 30 minutes, or a time period in the range of 5 minutes to 12 hours (e.g., 5, 10, 20, 30, 40, 50, or 60 minutes, or 1, 2, 3, 4, 5, 6, 7, 8, 9, 10, 11 or 12 hours) at room temperature, or 32° C., or a temperate in the range of 20-40° C. Then we detect a more pure sample that primarily contains cysteine. The prepared sample can be assayed with a variety of assays or methods, including but not limited to, HPLC, gas chromatography coupled mass spectroscopy (GC-MS), a nanorod-based assay (FIGS. 4 and 6-9), and a nanoelectronic device (FIG. 12).

As HPLC and GC-MS are well-known techniques routinely used by one of ordinary skill in the art, one of ordinary skill in the art would have known how to tailor the HPLC or GC-MS settings according to the specific properties of samples, equipment, and analysis purpose (see for example, Steele et al., Anal Biochem. (2012) 429:45-52; Buckpitt et al., Anal Biochem. (1977) 83:168-77; Hartleb et al., Biomed Sci Appl. (2001) 764:409-43; Stabler et al., Anal Biochem. (1987) 162:185-96; Ubbink et al., Clin Chem. (1999) 45:670-5, all incorporated herein by reference as though fully set forth).

For the nanorod-based assay, the processed sample is mixed with gold nanorods or other types of nanorods as described herein, and is allowed to react for about 10, 20, or 30 minutes, or a time period in the range of 1 minute to 12 hours (e.g., 1, 2, 3, 4, 5, 10, 15, 20, 25, 30, 35, 40, 45, 50, 55, 60, 65, 70, 75 80, 85, 90, 95, 100, 105, 110, 115, or 120 minutes, or 1, 2, 3, 4, 5, 6, 7, 8, 9, 10, 11 or 12 hours) at room temperature, or 32° C., or a temperate in the range of 20-40° C. The nanorod concentration is in the range of about 100-500 nM (approximately 5×10¹⁰ particles per ml, or about 1, 2, 3, or 4×10¹⁰ particles per ml). Then we measure the change of absorption spectrum in the reacted sample.

The measured change of absorption spectrum can be a change in the absorbance at a certain wavelength (for example, about 600, 650, 700, 750, 800, 900, 950, 1000, or 1100 nm or a wavelength in the range of 600-1100 nm or 650-750 nm) during a certain time interval (for example, 1, 2, or 3 minutes, or a time interval in the range of 1-60 minutes). For example, after the HCl solution is added into the mixture of uncoated gold nanorods treated with CTAB, absorption spectrum of the reacted mixture is recorded with 1 cm path-length cell at 965 nm from 2 to 8 minutes, and a change in absorbance (as a factor of extinction) for the time interval is calculated. As compared to a lower cysteine level, a higher cysteine level leads to an elevation in extinction at 965 nm wavelength (FIGS. 4C and 4D).

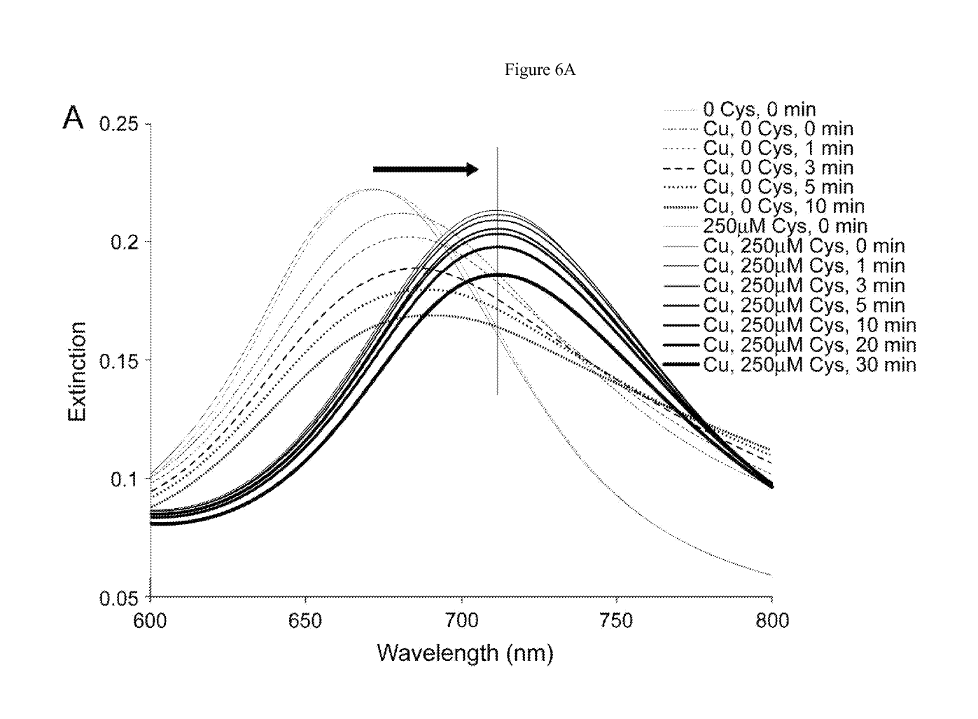

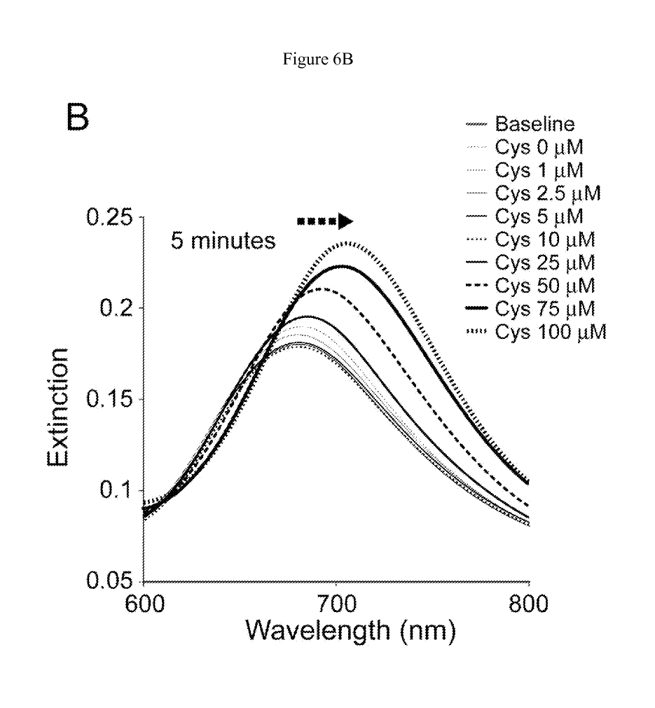

Furthermore, the measured change of absorption spectrum can be a change in the position of the absorption peak (i.e., the absorption peak wavelength). For example, one can use longitudinal surface protected nanorods with exposed gold transverse ends (i.e., polymer coated gold nanorods). An absorption spectrum for a wavelength range (for example, 650-750, 600-800, 500-900, or 400-1000 nm) can be recorded, and an absorption peak wavelength is determined from the recorded absorption spectrum. For example, after CuCl₂ is added, the absorption spectrum of the reacted mixture is recorded with 1 cm path-length cell at the whole range of 600-800 am, and the absorption peak wavelength is determined from the absorption spectrum. As compared to a lower cysteine level, a higher cysteine level moves the position of the absorption peak to a longer wavelength. As the cysteine level increases, the absorption peak wavelength increases (FIGS. 6-9). As the absorbance wavelength readings are stable 1 minute to greater than 30 minutes, this absorption peak based method is time-independent.

In various embodiments, a standard curve or a mathematical function can be generated from a series of cysteine standards, for example, 0, 25, 50, 100, 200, 300, 400, and 500 μM cysteine. Changes of absorption spectrum (for example, a change in the absorbance at a certain wavelength during a certain time interval, or a change in the position of the absorption peak of a certain wavelength range) are measured in correspondence with the series of cysteine standards, and a standard curve and/or a mathematical function depicting the relationship between cysteine standards and the measured changes of absorption spectrum is obtained. Then, the change of absorption spectrum is measured for a sample from a subject, and the cysteine level in the sample is determined using the standard curve and/or the mathematical function.

As examples, nanorods can be 30 nm by 10 nm and made of pure gold. Alternatively, nanorods can be made of selenium or cadmium with gold tips to improve detection sensitivity. Copper can be added to improve specificity.

### Nanorods and Systems

In various embodiments, the invention provides a system that comprises cystathionine synthase (e.g., cystathionine beta-synthase), cystathionine lyase (e.g., cystathionine gamma-lyase) and a nanorod. In accordance with the present invention, the system can be used to detect a cysteine level in a sample from a subject. In various embodiments, the detected cysteine level represents the total level of methionine metabolites including cystathionine, homocysteine and cysteine. Still in accordance with the present invention, the system can be used to predict the probability of a recurrence of a cancer in a subject. Also in accordance with the present invention, the system can be used to prescribe and/or administer an appropriate therapy to a subject.

In various embodiments, the cystathionine synthase is cystathionine beta-synthase. In various embodiments, the cystathionine lyase is a cystathionine gamma-lyase. In various embodiments, the cystathionine synthase is a polypeptide comprising a sequence as set forth in SEQ ID NO:1 or SEQ ID NO:5. In various embodiments, the cystathionine synthase is a polypeptide consisting of a sequence as set forth in SEQ ID NO:1 or SEQ ID NO:5. In various embodiments, the cystathionine lyase is a polypeptide comprising a sequence as set forth in SEQ ID NO:8 or SEQ ID NO:12. In various embodiments, the cystathionine lyase is a polypeptide consisting of a sequence as set forth in SEQ ID NO:8 or SEQ ID NO:12.

In accordance with the present invention, the nanorod comprises two end surfaces and a longitudinal surface. In various embodiments, the two end surfaces are reactive with cysteine. In various embodiments, the longitudinal surface is non-reactive with cysteine. In various embodiments, the nanorods can be made of gold, selenium, cadmium, copper, platinum, palladium, or carbon, or a combination thereof. In various embodiments, the nanorod is single layer carbon nanorod, multilayer carbon nanorod, or ordered mesoporous carbon nanorod. In various embodiments, the nanorod is a naked nanorod, or a coated nanorod, or a mixture thereof. In various embodiments, the naked nanorod is further protected with CTAB, perylene, or 16-mercaptohexadecyl trimethylammonium bromide (MTAB), or a combination thereof. In various embodiments, the longitudinal surface of the coated nanorod is coated with platinum, palladium, or selenium, or a combination thereof. In various embodiments, the longitudinal surface of the coated nanorod is coated with carboxybiphenyl-terminated polystyrene, polystyrene sulfonate (PSS), polyethylene glycol (PEG), methoxy PEG-thiol, or a combination thereof. Other examples include polyelectrolyte coatings with poly(diallyldimethylammonium chloride) (PDADMAC), poly(4-styrenesulfonic acid) (PSS), polyacrylic acid (PAA), poly(allylamine) hydrochloride (PAH), polyethyleneimine (pEI25). In various embodiments, the longitudinal surface of the coated nanorod is coated with carbon or an allotrope of carbon, or silicon. As an example, the allotrope of carbon can be grapheme or fullerene.

In further embodiments, the system can further comprise Cu²⁺. The concentration of Cu²⁺ is in the range of about 0.1-1 or 1-10 mM. In further embodiments, the system can further comprise HCl. The concentration of HCl is 0.01N or in the range of about 0.1-1 or 1-10 mM. In various embodiments, the system can further comprise serine. In various embodiments, the system can further comprise pyridoxal phosphate. In various embodiments, the system can further comprise a pH adjustment component for adjusting the pH to 5.5 or 5.0. In various embodiments, the system can further comprise a spin column with a molecular weight cutoff value at 2000, 3000, or 4000 Da. In various embodiments, the system can further comprise glutathione bound sepharose beads or cellulose resin.

In further embodiments, the system can further comprise an isolated sample from a subject. In various embodiments, the subject can be human, monkey, ape, dog, cat, cow, horse, goat, pig, rabbit, mouse or rat. In accordance with various embodiments of the present invention, the subject is suspected of having a cancer, has a symptom of a cancer, or is diagnosed with a cancer. In accordance with various embodiments of the present invention, the subject has received, is receiving, or will receive a cancer treatment. In accordance with various embodiments of the present invention, the subject is in complete or partial remission, or has a recurrence of cancer. In various embodiments, the recurrence can be biochemical recurrence. In various embodiments, the cancer can be prostate cancer, colon cancer, breast cancer, lung cancer, renal cancer, or bladder cancer.

In various embodiments, the isolated sample can be serum, urine, blood, plasma, saliva, semen, lymph, or a combination thereof. In various embodiments, the sample can be obtained before, during, or after cancer treatment. In some embodiments, the sample is urine and the urine cysteine level in the subject is above about 200, 210, 220, 230, or 240 micromoles of cysteine per milligram creatinine. In some embodiments, the sample is serum and the serum cysteine level in the subject is above about 400, 410, 420, 430, or 440 μM of cysteine.

In further embodiments, the system can further comprise a PSA test, clinical stage, biopsy Gleason score, pathologic Gleason score, pathologic stage, surgical margin status, lymph node involvement, or seminal vesicle involvement, or a combination thereof. PSA level, clinical stage, and biopsy Gleason score have pre-surgical predictive value. Post-surgical standard of care information such as pathologic Gleason score, pathologic stage, surgical margin status, lymph node involvement, and seminal vesicle involvement can also augment the use of cysteine quantitation. In various embodiments, the PSA test is a test of PSA velocity and/or total PSA level. PSA velocity means the rate at which PSA level rises over time.

In various embodiments, the invention provides a method of detecting a cysteine level in a sample from a subject. In various embodiments, the detected cysteine level represents the total level of methionine metabolites including cystathionine, homocysteine and cysteine. The method comprises: obtaining a sample from the subject; processing the sample with cystathionine synthase and cystathionine lyase; contacting the processed sample with nanorods; measuring a change of absorption spectrum of the sample; and detecting the cysteine level based upon the change of absorption spectrum.

In various embodiments, the change of absorption spectrum is a change in the absorbance at a wavelength. As an example, the sample can be processed with cystathionine synthase and cystathionine lyase for about 10, 20, or 30 minutes, or a time period in the range of 5 minutes to 12 hours (e.g., 5, 10, 20, 30, 40, 50, or 60 minutes, or 1, 2, 3, 4, 5, 6, 7, 8, 9, 10, 11 or 12 hours) at room temperature, or 32° C., or a temperate in the range of 20-40° C.; then the processed sample can be reacted with nanorods for about 10, 20, or 30 minutes, or a time period in the range of 1 minute to 2 hours (e.g., 10, 15, 20, 25, 30, 35, 40, 45, 50, 55, 60, 65, 70, 75 80, 85, 90, 95, 100, 105, 110, 115, or 120 minutes) at room temperature, or 32° C., or a temperate in the range of 20-40° C.: a change of absorbance at a certain wavelength (for example, 900, 950, 965, or 1000 nm or a wavelength in the range of 600-1000 nm) can be recorded for a certain time interval (for example, 1, 2, or 3 minutes, or a time interval in the range of 1-30 minutes). In various embodiments, a standard curve or a mathematical function can be generated from a series of cysteine standards, for example, 0, 25, 50, 100, 200, 300, 400, and 500 μM cysteine. Absorbance values corresponding to the series of cysteine standards are measured to determine absorbance changes corresponding to the series of cysteine standards, and a standard curve and/or a mathematical function depicting the relationship between cysteine standards and absorbance changes is obtained. Then, the absorbance change is measured for a sample from a subject, and the cysteine level in the sample is determined based upon the measured absorbance change and the standard curve and/or the mathematical function. Still in accordance with the invention, the method can further comprise providing or preparing a series of cysteine standards.

In various embodiments, the change of absorption spectrum is a change in the position of the absorption peak (i.e., the absorption peak wavelength). As an example, the sample can be processed with cystathionine synthase and cystathionine lyase for about 10, 20, or 30 minutes, or a time period in the range of 5 minutes to 12 hours (e.g., 5, 10, 20, 30, 40, 50, 60 minutes or 1, 2, 3, 4, 5, 6, 7, 8, 9, 10, 11 or 12 hours) at room temperature, or 32° C., or a temperate in the range of 20-40° C.; the processed sample is filtered and diluted 10-fold; then the processed sample can be reacted with nanorods for about 10, 20, or 30 minutes, or a time period in the range of 1 minute to 2 hours (e.g., 10, 15, 20, 25, 30, 35, 40, 45, 50, 55, 60, 65, 70, 75 80, 85, 90, 95, 100, 105, 110, 115, or 120 minutes) at room temperature, or 32° C., or a temperate in the range of 20-40° C.; an absorption spectrum for a wavelength range (for example, 650-750, 600-800, 500-900, or 400-1000 nm) can be recorded, and an absorption peak wavelength is determined from the absorption spectrum. In various embodiments, a standard curve or a mathematical function can be generated from a series of cysteine standards, for example, 0, 25, 50, 100, 200, 300, 400, and 500 μM cysteine. Absorption spectra corresponding to the series of cysteine standards are measured to determine absorption peak wavelengths corresponding to the series of cysteine standards, and a standard curve and/or a mathematical function depicting the relationship between cysteine standards and absorbance peak wavelengths is obtained. Then, the absorption spectrum is measured for a sample from a subject to determine the absorption peak wavelength for the sample, and the cysteine level in the sample is determined based upon the peak wavelength and the standard curve and/or the mathematical function. Still in accordance with the invention, the method can further comprise providing or preparing a series of cysteine standards.

### Nanoelectronic Devices and Systems

In various embodiments, the invention provides a nanoelectronic device that comprises: a first electrode with a first surface; a second electrode with a second surface; a hinge connecting the two electrodes, and an ammeter measuring the electric current flowing between the two electrodes. The two electrodes have different electric potentials. The hinge is non-conductive. The two surfaces face each other. The first surface is functionalized to bind cysteine, while the second surface is not functionalized to bind cysteine. The shape of the electrode can take a variety of shapes, including but not limited to, rod, sheet, plate, and disc. In accordance with the present invention, the nanoelectronic device can be used to detect a cysteine level in a sample from a subject. In various embodiments, the detected cysteine level represents the total level of methionine metabolites including cystathionine, homocysteine and cysteine. Still in accordance with the present invention, the nanoelectronic device can be used to predict the probability of a recurrence of a cancer in a subject. In further accordance with the present invention, the nanoelectronic device can be used to prescribe and/or administer an appropriate therapy to a subject.

In various embodiments, the invention provides a system that comprises a nanoelectronic device, cystathionine synthase, cystathionine lyase, and a linker. The linker is conductive, has at least one free thiol group, and has sufficient length to connect the two surfaces of the nanoelectronic device. Examples of the linker include but are not limited to a cysteine-functionalized nanoparticle, or a flexible molecule such as a secondary structured polypeptide and a secondary structured ssDNA, or a nanoparticle conjugated with a flexible molecule. In accordance with the present invention, the system can be used to detect a cysteine level in a sample from a subject. In various embodiments, the detected cysteine level represents the total level of methionine metabolites including cystathionine, homocysteine and cysteine. Still in accordance with the present invention, the system can be used to predict the probability of a recurrence of a cancer in a subject. In further accordance with the present invention, the system can be used to prescribe and/or administer an appropriate therapy to a subject.

Various embodiments of the system are shown in FIGS. 12A, B, and C. The nanoelectronic device comprises two nanoelectrodes 1201 and 1204. The two nanoelectrodes can have different electric potentials to produce a voltage between them. The nanoelectrode 1201 is a bare gold nanoelectrode with a surface 1202 capable of binding to cysteine or free thiol group (—SH), whereas the nanoelectrode 1204 has a surface 1203 that cannot bind to cysteine or free thiol group. The two surfaces face each other. The two electrodes are connected on one side with a hinge 1206 and the other side would be open. The hinge 1206 can be a butterfly type hinge. As the hinge 1206 is nonconductive, there is no current flowing between the two electrodes. A sample containing cysteine is contacted with the nanoelectronic device and cysteine in the sample binds to the surface 1202 until saturation. The sample is removed and the nanoelectronic device is washed. A conductive linker 1205 or 1208 or 1209 is contacted with the electronic device. The amount of current is measured after drying the excess liquid form the system. In FIG. 12A, the linker 1205 is nanoparticles (e.g., nanorods, nanospheres, nanofibers, nanowires, and nanotubes) functionalized to have free thiol group (—SH). After the link 1205 binds to the remaining unoccupied binding sites on the surface 1202, the nanoelectronic device is washed. As the functionalized particles have a length equal to the distance between the two electrodes, the functionalized particles connect the two electrodes and conduct an electric current flowing between the two electrodes. This electric current is measured by an ammeter. The current is inversely proportional to the amount of cysteine in the sample. In FIG. 12B, the linker 1208 is a flexible molecule having free thiol group (—SH). After the link 1208 binds to the remaining unoccupied binding sites on the surface 1202, the nanoelectronic device is washed. The pH and/or ionic strength in the system is changed to break hydrogen and/or ionic bonds in the flexible molecule. As a result, the flexible molecule becomes activated and opens up. As the activated flexible molecule has a length equal to the distance between the two electrodes, the activated flexible molecule connects the two electrodes and conducts an electric current flowing between the two electrodes. This electric current is measured by an ammeter. The current is inversely proportional to the amount of cysteine in the sample. In FIG. 12C, the linker 1209 is cysteine-bound nanoparticles (e.g., nanorods, nanospheres, nanofibers, nanowires, and nanotubes). Without free thiol group (—SH), these cysteine-bound nanoparticles do not bind to the remaining unoccupied binding sites on the surface. However, Cu²⁺ forms coordinate bonds with cysteine bound on the electrode and cysteine bound on the nanoparticles. As the cysteine-bound nanoparticles have a length equal to the distance between the two electrodes, the cysteine-bound nanoparticles connect the two electrodes and conduct an electric current flowing between the two electrodes. This electric current is measured by an ammeter. The current is directly proportional to the amount of cysteine in the sample.

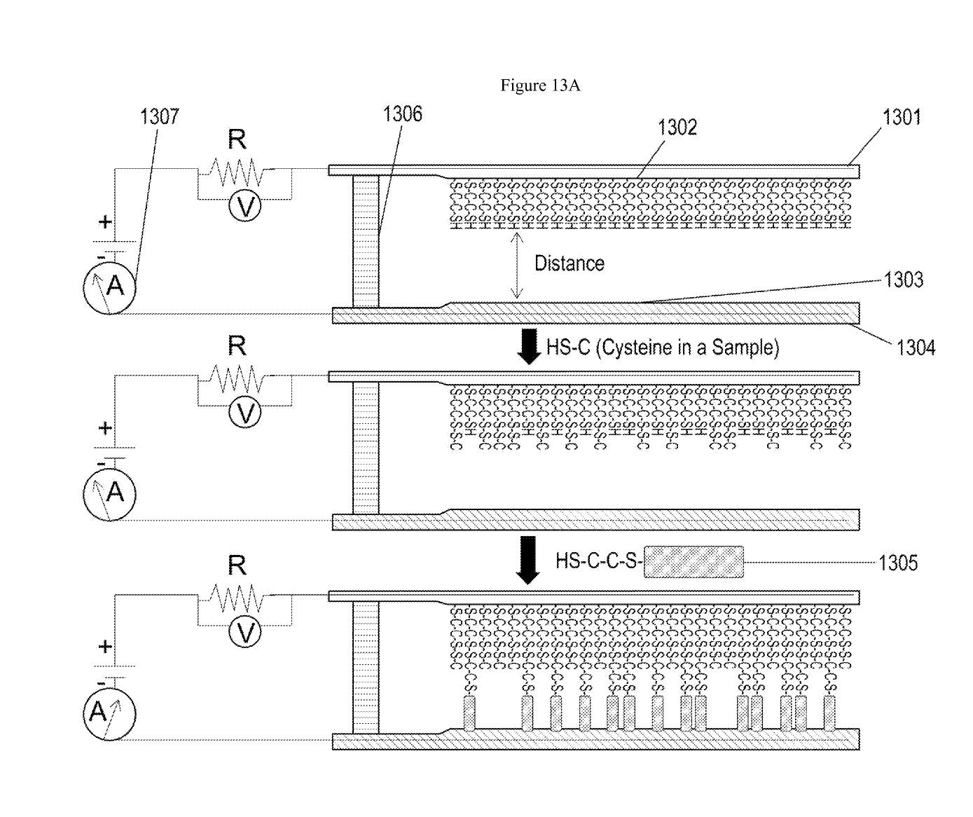

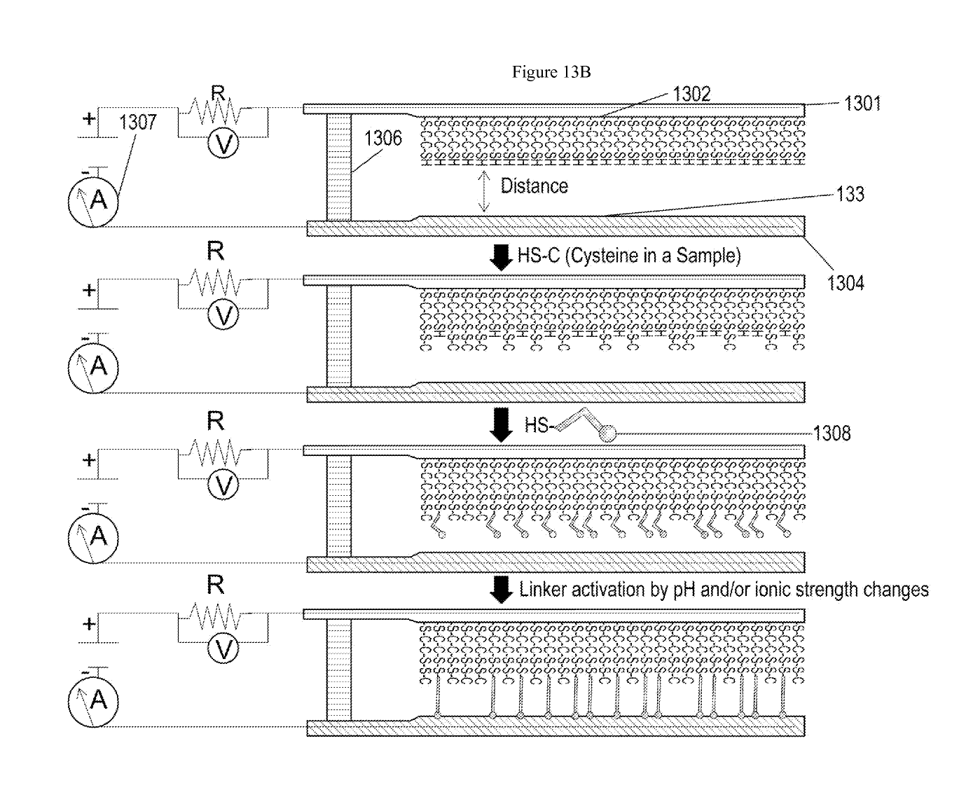

Various embodiments of the system are shown in FIGS. 13A and B. The nanoelectronic device comprises two nanoelectrodes 1301 and 1302. The two nanoelectrodes can have different electric potentials to produce a voltage between them. The nanoelectrode 1301 has a surface 1302 functionalized to have free thiol group (—SH) for binding to cysteine or another free thiol group (—SH), whereas the nanoelectrode 1304 has a surface 1303 that cannot bind to cysteine or free thiol group. The two surfaces face each other. The two electrodes are connected on one side with a hinge 1306 and the other side would be open. The hinge 1306 can be a butterfly type hinge. As the hinge 1306 is nonconductive, there is no current flowing between the two electrodes. A sample containing cysteine is contacted with the nanoelectronic device, and cysteine in the sample is induced to form a disulphide bond (—S—S—) with the free thiol group on the surface 1302 until saturation. The sample is removed and the nanoelectronic device is washed. A conductive linker 1305 or 1308 or 1309 is contacted with the electronic device. The amount of current is measured after drying the excess liquid form the system. In FIG. 13A, the linker 1305 is nanoparticles (e.g., nanorods, nanospheres, nanofibers, nanowires, and nanotubes) functionalized to have free thiol group (—SH). After the link 1305 forms disulphide bonds (—S—S—) with the remaining free thiol groups on the surface 1302, the nanoelectronic device is washed. As the functionalized particles have a length equal to the distance between the two electrodes, the functionalized particles connect the two electrodes and conduct an electric current flowing between the two electrodes. This electric current is measured by an ammeter. The current is inversely proportional to the amount of cysteine in the sample. In FIG. 13B, the linker 1308 is a flexible molecule having free thiol group (—SH). After the link 1308 forms disulphide bonds (—S—S—) with the remaining free thiol groups on the surface 1302, the nanoelectronic device is washed. The pH and/or ionic strength in the system is changed to break hydrogen and/or ionic bonds in the flexible molecule. As a result, the flexible molecule becomes activated and opens up. As the activated flexible molecule has a length equal to the distance between the two electrodes, the activated flexible molecule connects the two electrodes and conducts an electric current flowing between the two electrodes. This electric current is measured by an ammeter. The current is inversely proportional to the amount of cysteine in the sample.

In accordance with various embodiments of this invention, one of the two electrodes in the nanoelectronic device has a surface capable of binding to cysteine or a free thiol (—SH). Examples of this electrode include but are not limited to, bare gold nanoplates; gold nanoplates functionalized with free thiol groups (—SH); and other metallic nanoplates (e.g., selenium, cadmium, copper, platinum, palladium) or nonmetallic nanoplates (carbon, graphene, or fullerene) functionalized with free thiol groups (—SH). A nanoplate can be functionalized by being coated or conjugated with a molecule that has a free thiol group. After being functionalized, the nanoplate becomes capable of forming a disulphide bond with cysteine or free thiol group (—SH). A variety of molecules with a free thiol group can be used to cysteine-functionalization of a nanoparticle. Examples include, but are not limited to, cysteine itself. N-acetyl cysteine, homocysteine, cysteine-cysteine dipeptide, cysteine polypeptide, polypeptide with free cysteine at both ends, secondary structured polypeptide with free cysteine at both ends, dsDNA with cysteine residues at both ends, secondary structure containing ssDNA with cysteine residues at both ends, other synthetic molecules with cysteine residues at both ends (e.g., C₁₀₋₁₀₀, C₁₀₀₋₁₀₀₀, and C₁₀₀₀₋₁₀₀₀₀, long-chain saturated hydrocarbon, C₁₀₋₁₀₀, C₁₀₀₋₁₀₀₀, and C₁₀₀₀₋₁₀₀₀₀ long-chain unsaturated hydrocarbon polythene or biological polymers, C₁₀₋₁₀₀, C₁₀₀₋₁₀₀₀, and C₁₀₀₀₋₁₀₀₀₀ long-chain fatty acids, C₁₀₋₁₀₀, C₁₀₀₋₁₀₀₀, and C₁₀₀₀₋₁₀₀₀₀ long-chain carbohydrate etc.). In various embodiments, the molecule used for cysteine-functionalization of a nanoparticle can be a cysteine derivative containing a free thiol group, or R—SH, in which R can be a C₆₋₂₀ aryl group, a C₁₋₂₀ alkyl group, a C₂₋₂₀ alkynyl group, a C₂₋₂₀ alkenyl group, an aliphatic chain, an unsaturated aliphatic chain, or a saturated aliphatic chain. Various oxydising agents can induce formation of disulfide bonds, that includes Hydrogen peroxide (H₂O₂), Ozone (O₃), Flurine (F₂), Chlorine (Cl₂), Manganate (MnO₄²⁻), Permanganate (MnO₄⁻), Cromium trioxide (CrO₃), Chromate (CrO₄²⁻), Dichromate (Cr₂O₇²⁻) etc. Increase in temp also induces formation of disulfide bonds.

In some embodiments, the linker can be a flexible molecule with inactive and active statuses. In other embodiments, the linker can be a nanoparticle conjugated with a flexible molecule. In various embodiments, the nanoparticle is a nanorod, nanosphere, nanofiber, nanowire, or nanotube. In accordance with the present invention, the length of the inactive linker is insufficient to connect the two surfaces, and the length of the active linker is sufficient to connect the two surfaces. In various embodiments, the flexible molecule has free thiol group (—SH) for binding to a bare gold nanoplate or for form disulphide bond (—S—S—) with another free thiol group (—SH).

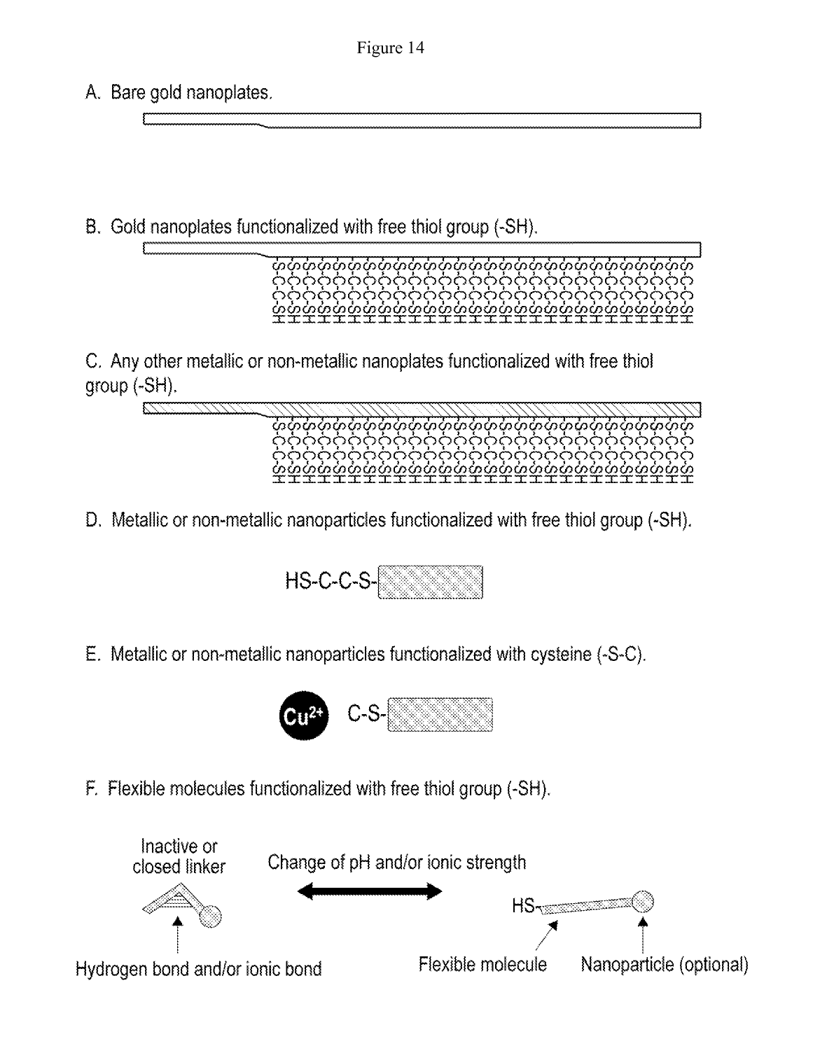

A flexible molecule is a type of molecule that can alter it length in different statuses. For example, the status and hence the length of a flexible molecule can be affected by pH and/or ionic strength. As an example, when changes in pH and/or ionic strength break hydrogen bond and/or ionic bond between parts of the flexible molecule, the flexible molecule opens up to take an active status with an increased length (FIG. 14F). Hydrogen bonds and ionic bonds are week bonds. Small changes in pH or ionic strength can be enough to activate a flexible molecule. One of ordinary skill in the art can vary the degree of changes in pH and/or ionic strength according to the type of flexible molecule in use. In general, acid pH (<4.5) or basic pH (>9.5) is not favorable for hydrogen bonds in biological macro-molecules. Ionic strength change for breaking ionic bond depends upon the nature of the molecule. Moderate heating of the system is one of the factors that can break both ionic and hydrogen bonds.

Examples of the flexible molecule include but are not limited to secondary structured polypeptides, secondary structured ssDNAs, and other C₁₀₋₁₀₀, C₁₀₀₋₁₀₀₀, and C₁₀₀₀₋₁₀₀₀₀ long-chain hydrocarbon compounds. The long chain portion gives the flexibility of the molecule. In some embodiments, the length of the activated flexible molecule (e.g., a very large macro-molecule) is sufficient to cover the distance between the two electrodes, and the flexible molecule can serve as a linker. In other embodiments, the flexible molecule is further conjugated to a nanoparticle to form a “flexible molecule-nanoparticle” complex as a bigger linker molecule. When the flexible molecule in the a bigger linker molecule is activated, this bigger linker molecule has sufficient length for covering the distance between the two electrodes.

In various embodiments, the linker can be a cysteine-functionalized nanoparticle having free thiol group (—SH). In various embodiments, the nanoparticle is a nanorod, nanosphere, nanofiber, nanowire, or nanotube. Nanoparticles are larger than amino acids, and hence the gap will help to maintain the voltage difference between two electrodes. A nanoparticle can be cysteine-functionalized by being coated or conjugated with a molecule that has a free thiol group. After being cysteine-functionalized, the nanoparticle becomes capable of binding to a bare gold nanoelectrode and forming a disulphide bond with cysteine or free thiol group (—SH). A variety of molecules with a free thiol group can be used to cysteine-functionalization of a nanoparticle. Examples include, but are not limited to, cysteine itself, N-acetyl cysteine, homocysteine, cysteine-cysteine dipeptide, cysteine polypeptide, polypeptide with free cysteine at both ends, secondary structured polypeptide with free cysteine at both ends, dsDNA with cysteine residues at both ends, secondary structure containing ssDNA with cysteine residues at both ends, other synthetic molecules with cysteine residues at both ends (e.g., C₁₀₋₁₀₀, C₁₀₀₋₁₀₀₀, and C₁₀₀₀₋₁₀₀₀₀ long-chain saturated hydrocarbon, C₁₀₋₁₀₀, C₁₀₀₋₁₀₀₀, and C₁₀₀₀₋₁₀₀₀₀ long-chain unsaturated hydrocarbon polythene or biological polymers, C₁₀₋₁₀₀, C₁₀₀₋₁₀₀₀, and C₁₀₀₀₋₁₀₀₀₀ long-chain fatty acids, C₁₀₋₁₀₀, C₁₀₀₋₁₀₀₀, and C₁₀₀₀₋₁₀₀₀₀ long-chain carbohydrate etc.). In various embodiments, the molecule used for cysteine-functionalization of a nanoparticle can be a cysteine derivative containing a free thiol group, or R—SH, in which R can be a C₆₋₂₀ aryl group, a C₁₋₂₀ alkyl group, a C₂₋₂₀ alkynyl group, a C₂₋₂₀ alkenyl group, an aliphatic chain, an unsaturated aliphatic chain, or a saturated aliphatic chain. Varoius oxydising agants can induce disulfide bonds, that includes Hydrogen peroxide (H₂O₂), Ozone (Os), Flurine (F₂), Chlorine (Cl₂), Manganate (MnO₄²⁻), Permanganate (MnO₄⁻), Cromium trioxide (CrO₃), Chromate (CrO₄²⁻), Dichromate (Cr₂O₇²⁻) etc. Increase in temp also induces formation of disulfide bonds.

In various embodiments, the linker can be a cysteine-bound nanoparticle without free thiol group (—SH). In various embodiments, the nanoparticle is a nanorod, nanosphere, nanofiber, nanowire, or nanotube. Many ions, including but limited to Cu²⁺, Ni²⁺, Zn²⁺, Hg²⁺, Pd²⁺, Pt²⁺, Co²⁺, Cd²⁺, and Ni²⁺, can form coordinate bonds with cysteine bound on the electrode and cysteine bound on the nanoparticles, thereby binding the linker and the electrode. The nanoparticle can be a nanorod, nanosphere, nanofiber, nanowire, or nanotube. Nanoparticles are larger than amino acids, and hence the gap will help to maintain the voltage difference between two electrodes.

In various embodiments, the cystathionine synthase is cystathionine beta-synthase. In various embodiments, the cystathionine lyase is a cystathionine gamma-lyase. In various embodiments, the cystathionine synthase is a polypeptide comprising a sequence as set forth in SEQ ID NO:1 or SEQ ID NO:5. In various embodiments, the cystathionine synthase is a polypeptide consisting of a sequence as set forth in SEQ ID NO:1 or SEQ ID NO:5. In various embodiments, the cystathionine lyase is a polypeptide comprising a sequence as set forth in SEQ ID NO:8 or SEQ ID NO:12. In various embodiments, the cystathionine lyase is a polypeptide consisting of a sequence as set forth in SEQ ID NO:8 or SEQ ID NO:12.

In further embodiments, the system can further comprise an isolated sample from a subject. In various embodiments, the subject can be human, monkey, ape, dog, cat, cow, horse, goat, pig, rabbit, mouse or rat. In accordance with the present invention, the subject is suspected of having a cancer, has a symptom of a cancer, or is diagnosed with a cancer. In accordance with the present invention, the subject has received, is receiving, or will receive a cancer treatment. In accordance with the present invention, the subject has a remission of a cancer, or has a recurrence of cancer. In various embodiments, the recurrence can be biochemical recurrence. In various embodiments, the cancer can be prostate cancer, colon cancer, breast cancer, lung cancer, renal cancer, or bladder cancer.

In various embodiments, the isolated sample can be serum, urine, blood, plasma, saliva, semen, lymph, or a combination thereof. In various embodiments, the sample can be obtained before, during, or after cancer treatment. In some embodiments, the sample is urine and the urine cysteine level in the subject is above about 200, 210, 220, 230, or 240 micromoles of cysteine per milligram creatinine. In some embodiments, the sample is serum and the serum cysteine level in the subject is above about 400, 410, 420, 430, or 440 μM of cysteine.

In further embodiments, the system can further comprise a PSA test, clinical stage, biopsy Gleason score, pathologic Gleason score, pathologic stage, surgical margin status, lymph node involvement, or seminal vesicle involvement, or a combination thereof. PSA level, clinical stage, and biopsy Gleason score have pre-surgical predictive value. Post-surgical standard of care information such as pathologic Gleason score, pathologic stage, surgical margin status, lymph node involvement, and seminal vesicle involvement can also augment the use of cysteine quantitation. In various embodiments, the PSA test is a test of PSA velocity and/or total PSA level. PSA velocity means the rate at which PSA level rises over time.

In various embodiments, the invention provides a method of detecting a cysteine level in a sample from a subject. In various embodiments, the detected cysteine level represents the total level of methionine metabolites including cystathionine, homocysteine and cysteine. The method comprises: obtaining a sample from the subject; processing the sample with cystathionine synthase and cystathionine lyase; contacting the processed sample to a nanoelectronic device; removing the processed sample; contacting a linker with the nanoelectronic device; measuring the electric current in the nanoelectronic device; and detecting the cysteine level based upon the measured electric current, wherein the measured electric current is representative of the cysteine level (either directly or inversely proportional depending upon the system). In some embodiments, a standard curve or a mathematical function can prepared from a series of cysteine standards, for example, 0, 25, 50, 100, 200, 300, 400, and 500 μM cysteine. The electric current generated on the nanoelectronic device corresponding to the series of cysteine standards are measured, and a standard curve and/or a mathematical function depicting the relationship between cysteine standards and electric currents is obtained. Then, the electric current is measured for a sample from a subject, and the cysteine level in the sample is determined based upon the measured electric current and the standard curve and/or the mathematical function. In other embodiments, based upon the standard curve and/or the mathematical function, the ammeter can be further labeled with a scale of cysteine concentrations, and can directly read out the cysteine level of a sample, without requiring further conversion of an electric current value into a cysteine concentration. Still in accordance with the invention, the system can comprise a series of cysteine standards. In various embodiments, the method can further comprise one or more steps of washing the electronic device. One or more steps of washing the electronic device can remove unbound linkers as so to improve the accuracy of the method.

### Methods and Treatments

In various embodiments, the present invention provides a method for preparing a sample for an assay to detect homocysteine, cystathionine, and cysteine level and a method of detecting homocysteine, cystathionine, and cysteine level in a sample from a subject. In various embodiments, the invention provides a method for preparing a sample for an assay to determine cysteine level. In various embodiments, the invention provides a method for detecting a cysteine level in a sample from a subject. In various embodiments, the detected cysteine level represents the total level of methionine metabolites including cystathionine, homocysteine and cysteine. In further embodiments, this method can be used to predict the probability of a recurrence of a cancer in a subject, and to prescribe and/or administer an appropriate therapy to a subject. The method comprises: obtaining a sample from the subject; processing the sample with cystathionine synthase and cystathionine lyase; and detecting a cysteine level in the processed sample using an assay to determine cysteine level.

In various embodiments, the subject can be human, monkey, ape, dog, cat, cow, horse, goat, pig, rabbit, mouse or rat. In accordance with the present invention, the subject is suspected of having a cancer, has a symptom of a cancer, or is diagnosed with a cancer, or prognosticated with a cancer. In accordance with the present invention, the subject has received, is receiving, or will receive a cancer treatment. In accordance with the present invention, the subject is in complete or partial remission, or has a recurrence of cancer.

In various embodiments, the isolated sample can be serum, urine, blood, plasma, saliva, semen, lymph, or a combination thereof. In various embodiments, the sample can be obtained before, during, or after cancer treatment. In some embodiments, the sample is urine and the urine cysteine level in the subject is above about 200, 210, 220, 230, or 240 micromoles of cysteine per milligram creatinine. In some embodiments, the sample is serum and the serum cysteine level in the subject is above about 400, 410, 420, 430, or 440 μM of cysteine.

In various embodiments, the cystathionine synthase is cystathionine beta-synthase. In various embodiments, the cystathionine lyase is a cystathionine gamma-lyase. In various embodiments, the cystathionine synthase is a polypeptide comprising a sequence as set forth in SEQ ID NO: 1 or SEQ ID NO:5. In various embodiments, the cystathionine synthase is a polypeptide consisting of a sequence as set forth in SEQ ID NO:1 or SEQ ID NO:5. In various embodiments, the cystathionine lyase is a polypeptide comprising a sequence as set forth in SEQ ID NO:8 or SEQ ID NO: 12. In various embodiments, the cystathionine lyase is a polypeptide consisting of a sequence as set forth in SEQ ID NO:8 or SEQ ID NO:12.

In further embodiments, the method further comprises predicting an increased probability of a recurrence of a cancer in the subject when the detected cysteine level in the subject is higher than a reference cysteine level. In accordance with the present invention, the reference cysteine level can be a mean or median cysteine level in non-recurrent subjects. In particular embodiments, the mean or media cysteine level is calculated from cysteine levels detected by a method, comprising: obtaining a sample from a subject; processing the sample with cystathionine synthase and cystathionine lyase; and detecting a cysteine level in the processed sample using an assay of cysteine level. In some embodiments, the detected cysteine level in the subject is at or about 5, 10, 15, 20, 25, 30, 35, 40, 45, 50, 55, 60, 65, 70, 75, 80, 85, 90, 95, or 100% higher than a reference cysteine level. In other embodiments, the detected cysteine level in the subject is at or about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 4-fold, 5-fold, 6-fold, 7-fold, 8-fold, 9-fold or 10-fold increase as compared to a reference cysteine level. In some embodiments, a reference cysteine level can be expressed in micromoles of cysteine per milligram creatinine for a sample, such as urine and serum samples. Examples of the reference cysteine levels in urine include, but not limited to values in the range of 140-579 nmol/mg creatinine. The reference cysteine level can be a value in the range of 160-220 micromoles of cysteine per milligram creatinine. Examples of the reference cysteine level include, but not limited to, 180, 190, 200, 210, or 220 micromoles of cysteine per milligram creatinine. In other embodiments, a reference cysteine level can be expressed in micromoles of cysteine a sample, such as urine, serum and other samples. Examples of the reference cysteine levels in serum include, but not limited to, values in the range of 200-370 μM. The reference cysteine level can be a value in the range of 320-380 μM cysteine. Examples of the reference cysteine level include, but not limited to, 340, 350, 360, 370, or 380 μM. The typical human reference ranges for urine creatinine are 30-300 mg/dl.

In various embodiments, the recurrence can be biochemical recurrence. In various embodiments, the cancer can be prostate cancer, colon cancer, breast cancer, lung cancer, renal cancer, or bladder cancer.

In various embodiments, the method further comprises: assessing at least one additional parameter; and predicting an increased probability of a recurrence of a cancer in the subject when the detected cysteine level in the subject is higher than a reference cysteine level and when the additional parameter in the subject is detected to be higher or lower than in non-recurrent subjects.

In some embodiments, the additional parameter is PSA velocity. PSA level, pre-surgical PSA level, post-surgical PSA level, pre-treatment PSA level, post-treatment PSA level, biopsy Gleason score, clinical stage, number of positive cores, number of negative cores, Karnofsky performance status, Hemoglobin value, Lactate dehydrogenase value, Alkaline phosphatase value, Albumin level, urinary albumin level, or urinary creatinine level, or a combination thereof. Urinary albumin level and urinary creatinine level can also be used to assess if the subject has good liver and kidney functions. Urinary creatinine level can also be used to normalize differences in urine volume when measuring urinary cysteine levels. In some further embodiments, the additional parameter is a pre-treatment parameter comprising pre-treatment PSA level, pre-treatment biopsy Gleason Score, pre-treatment clinical stage, pre-treatment urinary albumin level, or pre-treatment urinary creatinine level, or a combination thereof. In some embodiments, the PSA level in the subject is above about 6.0 ng/ml in serum. In some embodiments, the Gleason score in the subject is above 7.

In further embodiments, the method further comprises prescribing a first therapy to the subject, when the detected cysteine level in the subject is not higher than a reference cysteine level, or prescribing a second therapy or both the first therapy and the second therapy, when the detected cysteine level in the subject is higher than a reference cysteine level, wherein the first therapy is selected from the group consisting of active surveillance, prostatectomy, HIFU, cryotherapy and radio therapy, and wherein the second therapy is selected from the group consisting of systemic chemotherapy, hormonal therapy, pelvic floor salvage radiation. Still in accordance with the present invention, the method can further comprise treating the subject with the prescribed first therapy and/or second therapy.

In various embodiments, the assay to determine cysteine level comprises using HPLC. Examples of HPLC analysis methods include, but are not limited to, using radiation, fluorescence, or absorbance detection (Steele et al., Anal Biochem. (2012) 429:45-52; Buckpitt et al., Anal Biochem. (1977) 83:168-77; Hartleb et al., Biomed Sci Appl. (2001) 764:409-43)

In various embodiments, the assay to determine cysteine level comprises using GC-MS. Examples of GC-MS analysis methods include, but are not limited to, the use of radiolabeled tracers (Stabler et al., Anal Biochem. (1987) 162:185-96; Ubbink et al., Clin Chem. (1999) 45:670-5).

In various embodiments, the assay to determine cysteine level comprises using a nanorod. In various embodiments, the assay to determine cysteine level comprises using a nanoelectronic device. In various embodiments, the assay to determine cysteine level comprises using a system and the system comprises: cystathionine synthase, cystathionine lyase, and a nanorod. In various embodiments, the assay to determine cysteine level comprises using a system, and the system comprises: cystathionine synthase; cystathionine lyase; a nanoelectronic device; and a linker. The nanoelectronic device comprises: a first electrode with a first surface; a second electrode with a second surface; a hinge connecting the two electrodes; and an ammeter measuring the electric current flowing between the two electrodes. The two electrodes have different electric potentials. The hinge is non-conductive. The two surfaces face each other. The first surface is functionalized to bind cysteine, while the second surface is not functionalized to bind cysteine. The linker is conductive, has at least one free thiol group, and has sufficient length to connect the two surfaces of the nanoelectronic device.

### Polypeptides

In various embodiments, the present invention provides a polypeptide encoded by the sequence as set forth in SEQ ID NO:2. In various embodiments, the present invention provides a polypeptide consisting of the sequence as set forth in SEQ ID NO:5. In various embodiments, the present invention provides a polypeptide encoded by the sequence as set forth in SEQ ID NO:9. In various embodiments, the present invention provides a polypeptide consisting of the sequence as set forth in SEQ ID NO:12. In accordance with the present invention, these polypeptides can be used for detecting a cysteine level in a sample from a subject. In various embodiments, the detected cysteine level represents the total level of methionine metabolites including cystathionine, homocysteine and cysteine. In further embodiments, these polypeptides can be used to predict the probability of a recurrence of a cancer in a subject, and to prescribe and/or administer an appropriate therapy to a subject. In other embodiments, the polypeptides can contain a mutation, a deletion, an insertion, or a fusion, or a combination thereof.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Urine and serum samples (n=54 and 58, respectively), collected at the time of prostatectomy were divided into subjects who developed biochemical recurrence within 2 years and those who remained recurrence-free after 5 years. Multiple methionine metabolites were measured in urine and serum by GC-MS. The role of serum metabolites and clinical variables (biopsy Gleason grade, clinical stage, serum prostate specific antigen [PSA]) on biochemical recurrence prediction were evaluated. Urinary sarcosine and cysteine levels were significantly higher (p=0.03 and p=0.007 respectively) in the recurrent group. However, in serum, concentrations of homocysteine (p=0.003), cystathionine (p=0.007) and cysteine (p<0.001) were more abundant in the recurrent population. The inclusion of serum cysteine to a model with PSA and biopsy Gleason grade improved prediction over the clinical variables alone (p<0.001).

Higher serum homocysteine, cystathionine, and cysteine concentrations independently predicted risk of early biochemical recurrence and aggressiveness of disease in a nested case control study. The methionine metabolites further supplemented known clinical variables to provide superior sensitivity and specificity in multivariable prediction models for rapid biochemical recurrence following prostatectomy.

### Example 2

### A. Ethics Statement

This nested case-control study was conducted in accordance with the Institutional Review Board of Vanderbilt University. Written consent was given by the patients for their information to be stored in the hospital database. The board specifically approved the research use of the di-identified information and “on the shelf” specimens to be used for research under a waiver of consent.

### B. Patient Selection

The digital medical records of 400 subjects were retrospectively examined using the Vanderbilt University Department of Urologic Surgery registry of radical prostatectomies performed between 2003 and 2007. Several patients were excluded for reasons of compromised renal, heart, or liver function as was determined by electronic records of elevated urinary creatinine, hypertension, cardiac infarction history, and blood markers for hepatic function. Additionally, availability of follow-up data and records of pre-surgical hormone-ablation therapy were reasons for exclusion. Rapidly recurrent patients were identified as those who developed biochemical recurrence following prostatectomy within 2 years (American Joint Committee on Cancer defined as having PSA≧0.2 ng/ml, confirmed at least once two weeks later). The recurrence-free population was defined as having maintained a serum PSA<0.01 ng/ml for five or more years following surgery. Ultimately, for this nested case control study we focused on 54 subjects for analysis of urine and 58 subjects for analysis of serum who developed rapid biochemical recurrence and an age-matched recurrence-free control group who were free of recurrence. The mean age for the subjects was 60 years. All subjects were annotated based on age, pre-surgical serum PSA, biopsy Gleason score, clinical stage, and detection of biochemical recurrence.

### C. Urine and Serum Quantitative Metabolic Analysis

Serum and urine obtained at the time of radical prostatectomy were rapidly processed and stored at −80° C. We evaluated serum and urine for the metabolites, sarcosine, dimethylglycine, methionine, homocysteine, cystathionine, cysteine, methylmalonic acid and methylcitrate by gas-liquid chromatography/mass spectrometry [11,12,13]. Folate was measured microbiologically as described by Horne [14]. Urinary metabolites were expressed as nmol/mg creatinine to correct for differences in urine volume. Creatinine in urine was measured by the Jaffe method [15].

### D. Statistical Analysis

Patients' baseline demographic and clinical variables were assessed using Wilcoxon rank sum tests for continuous variables and Fisher exact tests for categorical (including binary) variables. All marker values, as well as PSA levels, were logarithmically transformed to achieve normality. Correlations among the markers were assessed using Spearman's rank correlation. Logistic regression models were used to analyze incidence of recurrence. The base model includes serum PSA, biopsy Gleason score, and clinical stage, clinical variables that are available prior to surgery. The post-surgical variables (e.g., lymph nodes, surgical margins, pathologic Gleason scores) were not considered. For multiplicity control, p≦0.007 (p-value less than 5%/7=0.7%) was considered statistically significant. To avoid further overfitting of the data, no variable selection was performed in the subsequent analyses based on logistic regression models. We used a likelihood ratio test to compare the simpler model (without the metabolites) and the full model (with the individual metabolites). Receiver operating characteristics (ROC) curves were generated for each logistic regression model, where the area under ROC curve (AUC) was determined. Integrated discrimination improvement (IDI) and Net reclassification index (NRI) [16] were used to compare the models' ability to distinguish recurrence and non-recurrence. The logrank tests were used to assess the difference in recurrence-free survival between the two groups illustrated by Kaplan-Meier plots. For the selected markers, Cox proportional hazard regression models were fit, and likelihood ratio tests were used to assess markers' association with time to recurrence outcome. The proportional hazard assumption was assessed using the method of Grambsch and Therneau [17]. All data analyses were performed using R 2.10.1 (R Development Core Team, Vienna, Austria); a significance level of 0.05 was used for statistical inference unless otherwise noted.

### E. Enzymes and Protein Sequences

An example of protein sequence of the cystathionine beta-synthase is SEQ ID NO:1 (Protein: Cystathionine beta-synthase 305 amino acids; Source organism: Helicobacter pylori 908; ACCESSION: ADN79248).

MILTAMQDAIGRTPIFKFTRKDYPIPLKSAIYAKLEHLNPGGSVKDRLGQ
YLIKEARTHKITSTTTIIEPTAGNTGIALALVAIKHHLKTIFVVPEKFSV
EKQQIMRALGALVINTPTSEGISGAIKKSKELAESIPDSYLPLQFENPDP
AAYYHTLAPEIVKELGTNFTSFVAGIGSGGTFAGTAKYLKERIPNIRLIG
VEPEGSILNGGEPGPHEIEGIGVEFIPPFFANLDIDGFETISDEEGFSYT
RKLAKKNGLLVGSSSGAAFAAALKEVQRLPEGSQVLTIFPDMADRYLSKG
IS

An optimized cystathionine beta-synthase (oCBS, 404 amino acids) could also be used. The optimized enzyme is constructed with codon usage enabling high E. coli expression and the addition of a cellulose binding domain for ease of purification with cellulose. The cellulose also can serve as a solid substrate for enzyme reaction.

oCBS nucleotide sequence (1215 bp; SEQ ID NO:2):

ATGACCCCGGTGTCTGGCAACCTGAAAGTCGAATTTTACAACTCCAAATC
CGTCTGATACCACGAATAGCATTAACCCGCAGTTCAAAGTTACGAACACC
GGCAGCTCTGCGATTGATCTGTCAAAACTGACGCTGCGTTATTACTATAC
CGTCGATGGTCAGAAAGACCAAACCTTTTGGTGCGACCATGCGGCCATTA
TCGGTAGTAACGGCTCCTACAATGGCATTACGTCTAATGTCAAAGGCACC
TTCGTGAAAATGAGTTCCTCAACGAACAATGGCGCCGGTGCAGGCGCTAT
GATCCTGACCGCGATGCAGGATGCCATCGGCCGTACGCCGATTTTTAAAT
TCACCCGCAAAGACTACCCGATCCCGCTGAAAAGTGCAATTTATGCTAAA
CTGGAACATCTGAATCCGGGCGGCAGCGTGAAAGATCGTCTGGGTCAATA
TCTGATTAAAGAAGCCTTTCGCACGCACAAAATCACCAGCACCACGACCA
TTATCGAACCGACGGCGGGTAATACCGGTATCGCACTGGCCCTGGTTGCC
ATTAAACATCACCTGAAAACCATCTTTGTGGTTCCGGAAAAATTCTCAGT
CGAAAAACAGCAAATCATGCGTGCGCTGGGCGCCCTGGTGATCAACACGC
CGACCTCAGAAGGTATCTCGGGCGCAATTAAAAAATCGAAAGAACTGGCT
GAAAGCATTCCGGATTCTTACCTGCCGCTGCAATTTGAAAACCCGGACAA
TCCGGCAGCTTACTATCATACCCTGGCACCGGAAATTGTGAAAGAACTGG
GCACGAATTTTACCAGCTTCGTTGCTGGTATCGGCTCTGGCGGTACGTTC
GCAGGCACCGCTAAATATCTGAAAGAACGTATTCCGAACATCCGCCTGAT
TGGCGTGGAACCGAAGGTAGTATTCTGAATGGCGGTGAACCGGGTCCGCA
CGAAATCGAAGGTATTGGCGTTGAATTTATCCCGCCGTTTTTCGCCAACC
TGGATATTGACGGCTTTGAAACGATTTCAGATGAAGAAGGTTTCTCGTAT
ACCCGCAAACTGGCGAAGAAAAACGGTCTGCTGGTTGGCAGCAGCAGCGG
TGCAGCATTTGCAGCTGCGCTGAAAGAAGTTCAGCGTCTGCCGGAAGGCA
GCCAGTCCTGACCATTTTCCCCGGATATGGCGGACCGCTACCTGAGTAAA
GGTATCTATTCCTAA

In SE ID NO:2, the linker is SEQ ID NO:3, which is bp 280-297 of SEQ ID:2:

GGCGCCGGTGCAGGCGCT

In SEQ ID NO:2, the Cellulose Binding Domain is SEQ ID NO: 4, which is bp 1-279 of SEQ ID:2:

ATGACCCCGGTGTCTGGCAACCTGAAAGTCGAATTTTACAACTCCAATCC
GTCTGATACCACGAATAGCATTAACCCGCAGTTCAAAGTTACGAACACCG
GCAGCTCTGCGATTGATCTGTCAAAACTGACGCTGCGTTATTACTATACC
GTCGATGGTCAGAAAGACCAAACCTTTTGGTGCGACCATGCGGCCATTAT
CGGTAGTAACGGCTCCTACAATGGCATTACGTCTAATGTCAAAGGCACCT
TCGTGAAAATGAGTTCCTCAACGAACAAT

oCBS protein sequence (404 amino acids. SEQ ID NO:5):

MTPVSGNLKVEFYNSNPSDTTNSINPQFKVTNTGSSAIDLSKLTLRYYYT
VDGQKDQTFWCDHAAIIGSNGSYNGITSNVKGTFVKMSSSTNNGAGAGAM
ILTAMQDAIFRTPIFKFTRKDYPIPLKSAIYAKLEHLNPGGSVKDRLGQY
LIKEAFRTHKITSTTTIIEPTAGNTGIALALVAIKHHLKTIFVVPEKFSV
EKQQIMRALGALVINTPTSWGISGAIKKSKELAESIPDSYLPLQFENPFN
PAAYYHTLAPEIVKELGTNFTSFVAGIGSGGTFAGTAKYLKERIPNIRLI
GVEPEGSILNGGEPGPHEIEGIGVEFIPPFFANLDIDGFETISDEEGFSY
TRKLAKKNGLLVGSSSGAADAAALKEVQRLPEGSQVLTIFPDMADRYLSK
GIYS*

In SEQ ID NO:5, the linker is SEQ ID NO: 6, which is aa 94-99 of SEQ ID:5:

GAGAGA

In SEQ ID NO:5, the Cellulose Binding Domain is SEQ ID NO: 7, which is aa 1-93 of SEQ ID:5:

MTPVSGNLKVEFYNSNPSDTTNSINPQFKYTNTGSSAIDLSKLTLRYYYT
VDGQKDQTFWCDHAAIIGSNGSYNGITSNVKGTFVKMSSSTNN

An example of protein sequence of the cystathionine gamma-lyase is SEQ ID NO:8 (Protein: Cystathionine gamma-lyase 378 amino acids; Source organism: Helicobacter pylori 908; ACCESSION: ADN79247).

MQTKLIFGGISEDATTGAVSVPIYQASTYRQDAIGRHKGYEYSRSGNPTR
FALEELIADLEGGVKGFAFASGLAGIHAVFSLLQSGDHVLLGDDVYGGTF
RLFNKVLVKNGLSCTIIDTSDISQIKKAIKPNTKALYLETPSNPLLKITD
LAQCASVAKDHGLLTIVSNTFATPYCQNPLLLGADIVAHSGTKYLGGHSD
VVAGLVTTNNEALAQEIAFFQNAIGGVLGPQDSWLLQRGIKTLGLRMEAH
QKNALCVAEFLEKHPKVERVYYPGLPTHPNHELAKAQMRGFSFMLSFTLK
NDSEAALFVESLKFILGESLGGVESLVGIPALMTHACIPKEQREAAGRID
GLVRLSVGIEHEQDLLEDLEQAFAKIS

An optimized cystathionine gamma-lyase (oCGL, 477 amino acids) could also be used. The optimized enzyme is constructed with codon usage enabling high E. coli expression and the addition of a cellulose binding domain for ease of purification with cellulose. The cellulose also can serve as a solid substrate for enzyme reaction.

oCGL nucleotide sequence (1434 bp: SEQ ID NO: 9):

ATGCGCCGGTGTCTGGCAATCTGAAAGTGGAATTTTACAACAGCAACCCG
AGCGATACGACGAATAGCATCAACCCGCAGTTCAAAGTGACCAACACGGG
TAGCTCTGCGATTGATCTGTCTAAACTGACCCTGCGTTATTACTATACGG
TTGATGGCCAGAAAGACCAAACCTTTTGGTGCGACCATGCGGCCATTATC
GGTTCTAACGGCAGTTATAATGGTATCACCAGCAATGTGAAAGGCACGTT
CGTTAAAATGAGTTCCTCAACCAACAATGGCGCAGGTGCTGGCGCGATGC
AGACGAAACTGATTCATGGCGGTATCAGCGAAGATGCAACCACGGGTGCA
GTCTCGGTGCCGATTTACCAGGCCAGCACCTATCGTCAAGACGCAATCGG
TCGCCACAAAGGCTACGAATATTCGCGTAGCGGTAACCCGACGCGCTTTG
CACTGGAAGAACTGATTGCGGATCTGGAAGGCGGTGTGAAAGGCTTTGCC
TTCGCATCAGGTCTGGCAGGCATCCATGCTGTTTTCTCGCTGCTGCAAAG
CGGTGACCACGTCCTGCTGGGCGATGACGTGTACGGCGGCACCTTTCGCC
TGTTCAACAAAGTTCTGGTCAAAAATGGTCTGAGTTGTACCATTATCGAT
ACGTCCGACATTTCACAGATCAAAAAAGCGATTAAACCGAACACCAAAGC
CCTGTATCTGGAAACGCCGTCGAATCCGCTGCTGAAAATTACCGATCTGG
CCCAGTGCGCAAGCGTTGCTAAAGATCATGGCCTGCTGACGATCGTGGAT
AACACCTTTGCGACGCCGTACTGTCAAAATCCGCTGCTGCTGGGTGCGGA
TATTGTCGCCCATTCCGGCACCAAATATCTGGGCGGTCACTCAGACGTGG
TTGCCGGTCTGGTTACCACGAACAATGAAGCTCTGGCGCAGGAAATTGCG
TTTTTCCAAAACGCAATCGGCGGTGTGCTGGGTCCGCAGGATAGCTGGCT
GCTGCAACGTGGTATCAAAACCCTGGGCCTGCGCATGGAAGCGCATCAGA
AAAATGCACTGTGCGTTGCTGAATTTCTGGAAAAACACCCGAAAGTGGAA
CGTGTTTACTATCCGGGTCTGCCGACCCATCCGAACCACGAACTGGCCAA
AGCACAAATGCGCGGTTTTTCTGGCATGCTGAGTTTCACGCTGAAAAATG
ATTCTGAAGCAGCTCTGTTTGTGGAAAGTCTGAAACTGTTCATTCTGGGT
GAATCCCTGGGCGGTGTCGAATCACTGGTGGGCATTCCGGCACTGATGAC
CCATGCTTGTATCCCGAAAGAACAGCGTGAAGCGGCCGGTATTCGTGATG
GCCTGGTTCGCCTGTCTGTCGGCATCGAACACGAACAGGATCTGCTGGAA
GACCTGGAACAGGCGTTTGCGAAAATTAGTTAA

In SEQ ID NO:9, the linker is SEQ ID NO: 10, which is bp 280-297 of SEQ ID:9:

GGCGCAGGTGCTGGCGCG

In SEQ ID NO:9, the Cellulose Binding Domain is SEQ ID NO: 11, which is bp 1-279 of SEQ ID:9:

ATGACGCCGGTGTCTGGCAATCTGAAAGTGGAATTTTACAACAGCAACCC
GAGCGATACGACGAATAGCATCAACCCGCAGTTCAAAGTGACCAACACGG
GTAGCTCTGCGATTGATCTGTCTAAACTGACCCTGCGTTATTACTATACG
GTTGATGGCCAGAAAGACCAAACCTTTTGGTGCGACCATGCGGCCATTAT
CGGTTCTAACGGCAGTTATAATGGTATCACCAGCAATGTGAAAGGCACGT
TCGTTAAAATGAGTTCCTCAACCAACAAT

oCGL protein sequence (477 amino acids; SEQ ID NO: 12):

MVSYKCGVKDGTKNTIRATINTGTTPVNLSDIKVRYWFTSDGENNFVCDY
AAFGTDKVKKKIENSVPGADTYCEISVKGTFVKMSSSTNNGAGAGAMQTK
LIHGGISEDATTGAVSVPIYQASTYRQDAIGRHKGYEYSRSGNPTRFALE
ELIADLEGGVKGFAFASGLAGIHAVFSLLQSGDHVLLGDDVYGGTFRLFN
KVLVKNGLSCTIIDTSDISQIKKAIKPNTKYLGGHSDPSNPLLKITDLAQ
CASVAKDHGLLTIVDNTFATPYCQNPLLLGADIVAHSGTKYLGGHSDVVA
GLVTTNNEALAQEIAFFQNAIGGVLGPQDSWLLQRGIKTLKNDSEAALFV
ESLKLFILEFLEKHPLVERVYYPGLPTHPNHELAKAQMRGFSGMLSFTLK
NDSEAALFVESLKLFILGESLGGVESLVGIPALMTHACIPKEQREAAGIR
DGLVRLSVGIEHEQDLLEDLEQAFAKIS*

In SEQ ID NO: 12, the linker is SEQ ID NO: 13, which is aa 94-99 of SEQ ID: 12:

GAGAGA

In SEQ ID NO:12, the Cellulose Binding Domain is SEQ ID NO: 14, which is aa 1-93 of SEQ ID:12:

MVSYKCGVKDGTKNTIRATINIKNTFTTPVNLSDIKVRYWFSDGENNFVC
DYAAFGTDKVKKKIENSVPGADTYCEISVKGTFCKMSSSTNN

The enzymes are expressed in E. coli following induction with IPTG. The E. coli are lysed and inclusion bodies centrifuged. The pelleted inclusion bodies washed 6 times and are further lysed by sonication. The released enzymes are denatured with 1 M urea and dialyzed in pH 5.0 HEPES buffer with 10% glycerol. The dialyzed enzymes are purified with cellulose resin. The enzymes are eluted from the cellulose with ddH₂O.

### F. Nanorods

Examples of nanorods that can be used include, but are not limited to, the following:

(a) Naked nanorods, CTAB-protected naked gold nanorods, and their combinations (FIGS. 4C and 4D). One example of the aspect ratio of the nanorods is 3:1. The dimensions include, but are not limited to, 30 nm 10 nm, 75 nm×25 nm, 100 nm×25 nm, or 150 nm×25 nm. However, CTAB protection coating is non-covalent binding. The CTAB protection coating, is not exclusive to the longitudinal surface, but statistically it covers a greater percentage of the longitudinal surface.

(b) Coated gold nanorods, polymer-coated nanorods, inert metal-coated nanorods, and their combinations (FIGS. 6-11). The longitudinal surface of polymer-coated nanorods is covered by a water-soluble polymer, including carboxybiphenyl-terminated polystyrene. The polymer shell insures high solubility of the hybrid structures as well as limits aggregation in the presence of cysteine. Alternatively, inert metals such as platinum, palladium, and selenium coating of the longitudinal length of the gold rods can be used. The polymers and inert metals are covalently bound to the longitudinal surface of nanorods.

(c) Carbon nanorods with gold ends on the transverse (shorter) ends (FIG. 5). The carbon nanorods and gold ends are bond with covalent linkages.

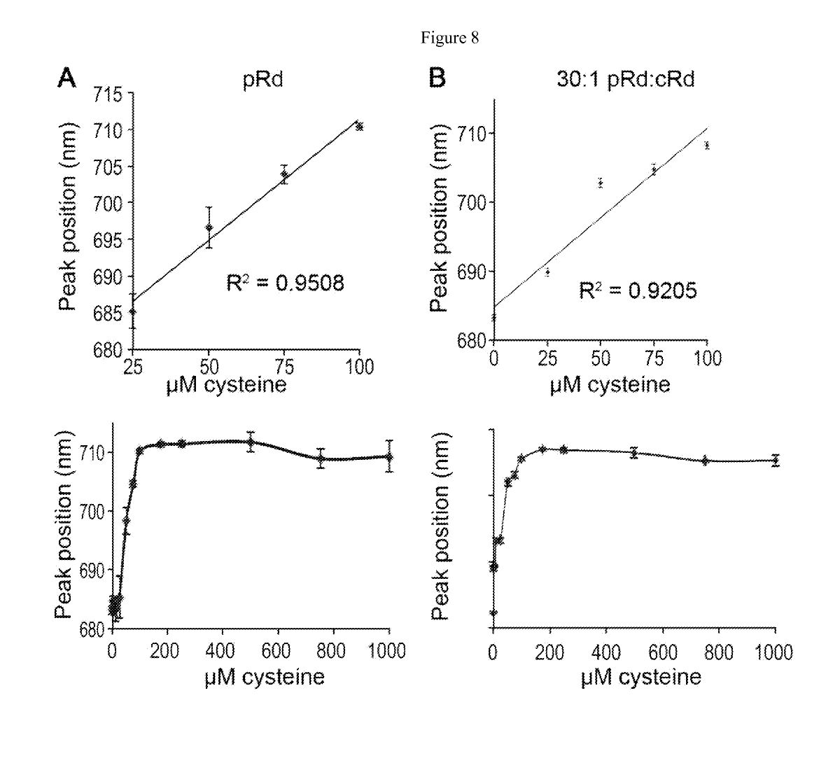

(d) A mixture of the rod types can be used. For example, a mixture of polymer-coated nanorods with CTAB protected naked nanorods with varying ratios can be employed to achieve improved sensitivity (FIG. 8B).

### G. Lime-Independent Detection

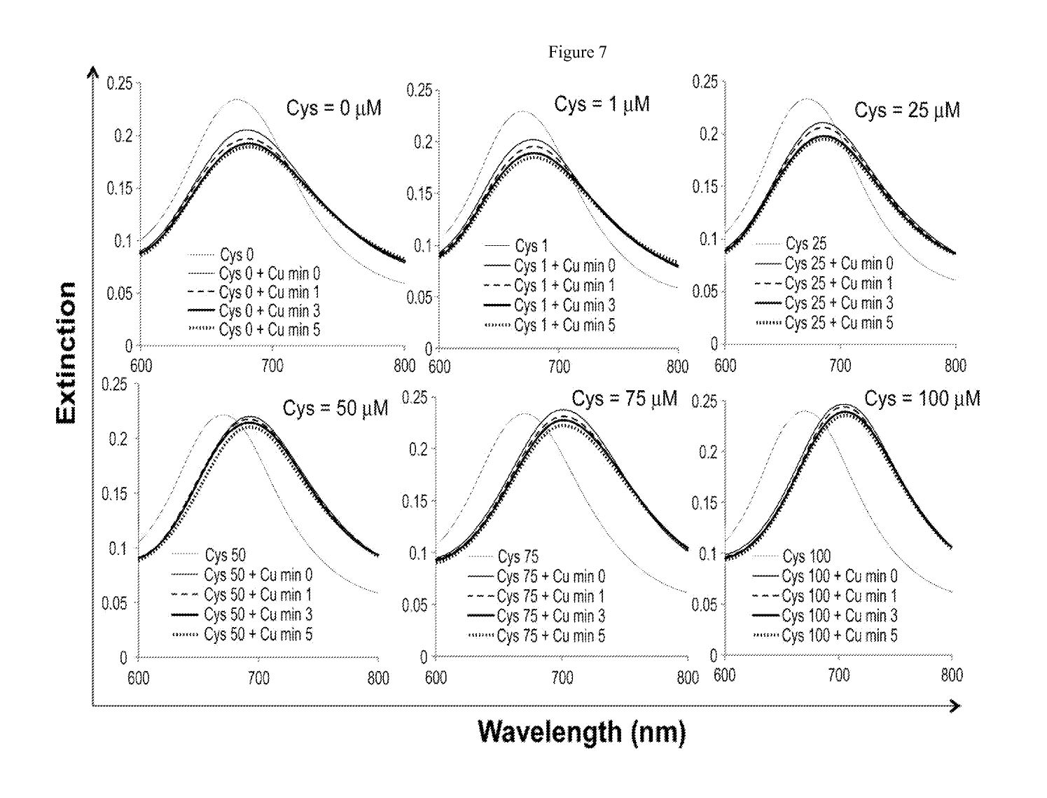

As a measure of robustness of the method and high throughput application, the absorbance readings of the rods following analyte interaction is stable 1 minute to greater than 30 minutes. FIGS. 6A-6B and FIG. 7 demonstrate stability of absorbance wavelength at different incubation times and concentrations of cysteine, respectively. This differs from other gold-nanorod based cysteine detection methods that depend on the absorbance changes at the 950 nm wavelength, where measurements need to be taken within a short time interval (FIGS. 4C and 4D). Further, when cysteine concentration is dependent on changes in absorbance at 950 nm, for different samples to be compared, the samples need to be read at the identical time interval following the introduction of the nanorods and CuCl₂.

### H. Cysteine Detection

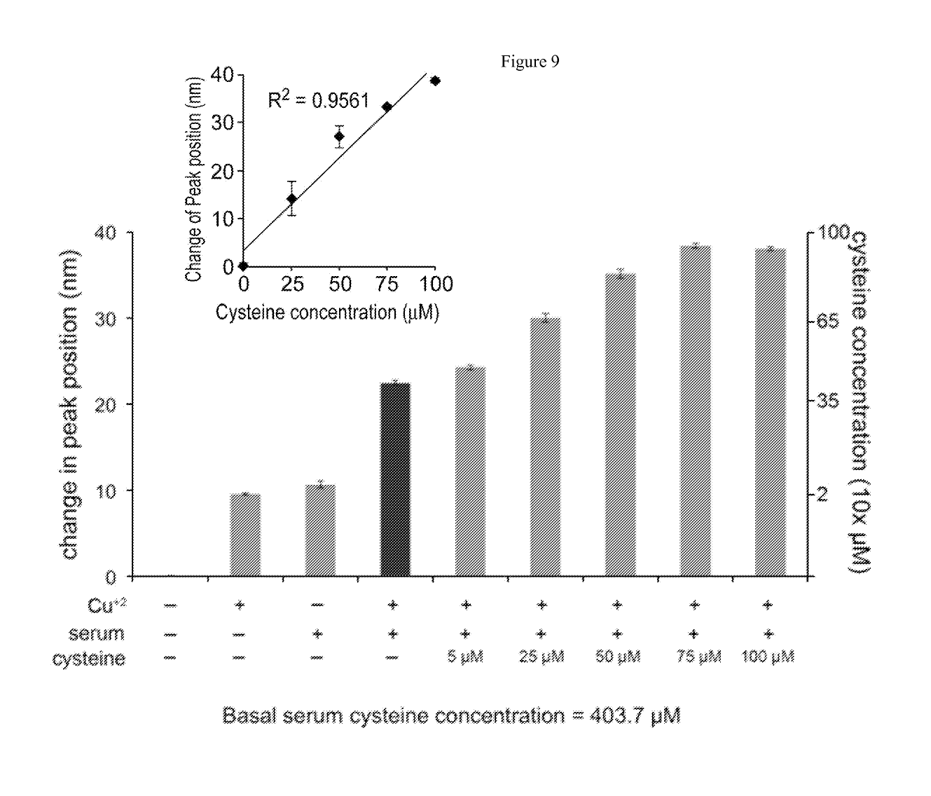

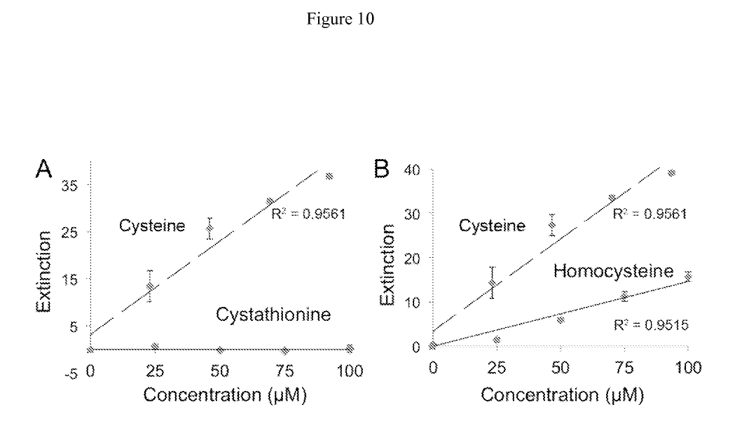

Serum: for detection of cysteine in serum (FIG. 9), 500 μl is required. (1) Urinary creatine and albumin levels are needed to determine eligibility for the test. Elevated urinary creatine and albumin (>1.2 mg/dL and >8 mg/dL, respectively) would exclude the use of the cysteine assay for the subject. 100 μl in triplicate is used for cysteine measurement. Thiol-dependent gold nanorod-based detection of cystathionine and homocysteine is limited, compared to that of cysteine (FIG. 10). (2) To enable efficient detection of cystathionine and homocysteine, the following will be added to each tube following a ten-fold dilution with water: serine, pyridoxal phosphate, cystathionine beta-synthase, and cystathionine gamma-lyase, and pH adjusted to 5.0 (FIG. 11). This reaction is allowed to proceed for 1 to 12 hours at 32° C. (3) The reaction is filtered through a 3000 Da molecular weight spin column at 10,000 rpm for 30 min. (4) The gold nanorods [100 pmol/ml, can replaced with other rod materials having Au ends] with an aspect ratio of 30 nm×10 nm (3:1) are added to the analyte and allowed to react for 30 min at room temperature. Importantly, if naked gold rods are used instead of alternative coated rods, the rods need to be protected with ceryltrimethylammonium bromide (CTAB) prior to analysis. Following incubation with the nanorods, CuCl_{2 [}0.2-1 mM] is added and the absorption spectra are recorded 600-800 nm wavelength. Readings can be had by 1 cm path length cuvette if samples are analyzed individually. High-throughput adaptation of the method can include a 96 well format. The above method can also be used for detection of cysteine in urine.

Urine: (1) 1 ml of urine is needed for the analysis. Creatine and albumin levels are measured using 200 μl for each assay. Elevated creatine and albumin (>1.2 mg/dL and >8 mg/dL, respectively) would exclude the use of the cysteine assay for the subject. (2) Of the remaining 600 μl, 200 μl in triplicate is used for cysteine measurement. To each of the tubes, the following will be added: serine, pyridoxal phosphate, cystathionine beta-synthase, and cystathionine gamma-lyase. This reaction is allowed to proceed for 20 min at room temperature. (3) Since the recombinant enzymes (of Helicobacter pylori) have a glutathione S transferase (GST) tag modification, glutathione bound sepharose beads (10 μl) is added to the reaction. Following 5 min. incubation on ice, the tubes are centrifuged briefly. The supernatant (free of the modifying enzymes) is transferred to wells of a 96 well plate. (4) Gold nanorods [10 μM, can replaced with SeCd rods having Au ends] with an aspect ratio of 10 nm×30 nm are added to the analyte and allowed to react for 10 min at room temperature. Importantly, if gold is used instead of SeCd rods, the rods need to be protected with cetyltrimethylammonium bromide (CTAB) prior to analysis. Following the 10 min incubation with the nanorods, HCl [0.2 mM] is added. The absorption spectra is recorded on a 96 well plate reader with dynamically from 2 min to 8 min. following HCl addition at 950 nm wavelength. Similar readings can be had by 1 cm path length cuvette if samples are analyzed individually. The above method can also be used for detection of cysteine in serum.

### Example 3

Methionine metabolites support prediction of biochemical recurrence—Urine metabolites were initially measured in fifty-four patients who developed biochemical recurrence (N=25) and those that remained recurrence-free (N=29). These patients were matched for age and pre-surgical serum PSA. Table 1 enumerates the clinical characteristics of the two patient groups by serum PSA, clinical stage, and biopsy Gleason grade. Majority of patients had a clinical stage of T1. Creatinine-normalized urinary dimethylglycine and homocysteine were not significantly different between the two groups. However, we found urinary sarcosine to be significantly elevated at the time of surgery in patients who developed biochemical recurrence, as originally reported for patients with frank prostate metastatic lesions [8]. We further found that urinary cysteine was significantly elevated in biochemically-recurrent patients compared to those who remained recurrence-free five years following prostatectomy. Urine analysis in a pre-surgical patient population suggested products of methionine catabolism might correlate with prostate cancer progression status.

Table 1: The values for methionine metabolites measured in the urine of the recurrent-free and the recurrent groups are compared. Values for sarcosine, homocysteine, dimethylglycine and cysteine are expressed as μmoles/mg creatinine. Wilcoxon rank sum tests for continuous variables and Fisher exact tests for categorical (including binary) variables are indicated. Normal values for metabolites (pmole/mg creatinine) are: cysteine, 140-579; homocysteine, 0.974-7.17; dimethylglycine, 10.1-108.2 and sarcosine. 2.65-8.67. Median values with quartiles were used to summarize the distributions of the continuous variables.

TABLE 1

| | Recurrent-free (29) | | Recurrent (25) | | P value |
|---|---|---|---|---|---|
| Age | 59 | (53, 64) | 62 | (58, 67) | 0.10 |
| Pre-surgery PSA | 5.2 | (4.3, 6.5) | 6.0 | (5.0, 8.2) | 0.08 |
| Clinical stage | | | | | 0.09 |
| (N = 16/18) | | | | | |
| T1 | 15 | (94%) | 12 | (67%) | |
| T2 | 1 | (6%) | 6 | (33%) | |
| T3 | 0 | 0 | | | |
| Biopsy Gleason | | | | | 0.050 |
| (N = 16/18) | | | | | |
| 4 | 1 | (6%) | 0 | | |
| 5 | 2 | (12%) | 0 | | |
| 6 | 9 | (56%) | 4 | (22%) | |
| 7 | 3 | (19%) | 8 | (44%) | |
| 8 | 1 | (6%) | 3 | (17%) | |
| 9 | 0 | | 2 | (11%) | |
| 10 | 0 | | 1 | (6%) | |
| Urine cysteine | 190 | (168, 212) | 221 | (189, 252) | 0.007 |
| (N = 29/24) | | | | | |
| Urine homocysteine | 2.7 | (2.2, 3.2) | 2.8 | (2.4, 4.0) | 0.40 |
| Urine | 27.3 | (22.1, 38.5) | 25.4 | (17.6, 33.7) | 0.34 |
| dimethylglycine | | | | | |
| Urine sarcosine | 3.7 | (3.1, 5.7) | 5.4 | (4.1, 6.7) | 0.03 |

We then performed a nested case control study with pre-surgical serum. Fifty-eight age-matched prostatectomy patients were stratified by pre-surgical PSA, clinical stage, and biopsy Gleason grade as well as pathologic variables (Table 2). As expected, clinical variables were significantly different in the two populations, as were the post-surgical pathologic factors. Interestingly, the serum homocysteine, cystathionine, and cysteine were significantly higher in the biochemically-recurrent patients (p value<0.001). However, clinical stage and serum levels of sarcosine, dimethylglycine, folate, methylcitrate, and methylmalonic acid were not significantly different between the two populations. Normal methylcitrate levels in both populations supported renal sufficiency. Serum methylmalonic acid levels, an indicator of vitamin B-12 status [18], were not different between the two groups. Serum and urine cysteine correlation did not reach statistical significance (p=0.06, Table 3). However, serum homocysteine was strongly correlated with cysteine (Spearman's rank correlation=0.65, p<0.01). Therefore, the higher serum homocysteine was not a function of differences in renal function, vitamin B-12 or folate status.

Table 2: The values for methionine metabolites measured in the sera of the recurrent-free and the recurrent groups are compared. Wilcoxon rank sum tests for continuous variables and Fisher exact tests for categorical (including binary) variables are indicated. Normal values for metabolites are: cysteine, 203-369 μM homocysteine, 5.4-13.9 μM; dimethylglycine, 1.4-5.3 μM; sarcosine, 0.6-2.7 μM; methionine, 11.3-42.7 μM; folate, >3.0 ng/ml; methylcitrate, 60-228 nM; methylmalonate, 73-271 nM; cystathionine, 44-342 nM. Median values with quartiles were used to summarize the distributions of the continuous variables.

TABLE 2

| | Recurrent-free (30) | | Recurrent (28) | | P value |
|---|---|---|---|---|---|
| Age | 59 | (54, 64) | 61 | (59, 64) | 0.07 |
| Pre-surgery PSA | 5.4 | (4.0, 8.1) | 6.8 | (5.2, 8.9) | 0.02 |
| Clinical stage | | | | | 0.30 |
| T1 | 24 | (80%) | 18 | (64%) | |
| T2 | 6 | (20) | 9 | (32%) | |
| T3 | 0 | | 1 | (4%) | |
| Biopsy Gleason | | | | | 0.006 |
| 4 | 1 | (3%) | 0 | | |
| 5 | 2 | (7%) | 0 | | |
| 6 | 18 | (60%) | 6 | (20%) | |
| 7 | 6 | (20%) | 13 | (46%) | |
| 8 | 2 | (7%) | 4 | (15%) | |
| 9 | 1 | (3%) | 4 | (15%) | |
| 10 | 0 | | 1 | (4%) | |
| Serum cysteine | 346 | (321, 377) | 419 | (367, 452) | <0.001 |
| Serum homocysteine | 9.0 | (8.0, 10.2) | 11.7 | (9.4, 13.4) | 0.003 |
| Serum dimethylglycine (n = 27/23) | 4.6 | (3.8, 4.7) | 4.9 | (4.2, 5.4) | 0.21 |
| Serum sarcosine (n = 27/23) | 1.3 | (1.1, 1.4) | 1.3 | (1.1, 1.7) | 0.67 |
| Serum methionine (n = 27/27) | 24.8 | (21.7, 30.6) | 27.6 | (23.9, 33.7) | 0.08 |
| Serum folate (n = 27/28) | 44.8 | (25.2, 52.8) | 42.3 | (31.3, 51.5) | 0.72 |
| Serum methylcitrate | 126 | (102, 144) | 135 | (117, 167) | 0.13 |
| Serum methylmalonate | 167 | (145, 220) | 164 | (146, 211) | 0.91 |
| Serum cystathionine (n = 29/26) | 149 | (130, 176) | 186 | (148, 239) | 0.007 |
| Lymph node involvement | 0 | (0%) | 6 | (21%) | 0.01 |
| SV involvement | 0 | (0%) | 8 | (29%) | 0.002 |
| Positive surgical margin | 1 | (3%) | 8 | (29%) | 0.01 |
| Stage III+ | 3 | (10%) | 21 | (75%) | <0.001 |
| Pathologic Gleason | | | | | 0.002 |
| 5 | 2 | (7%) | 0 | (0%) | |
| 6 | 15 | (50%) | 4 | (14%) | |
| 7 | 10 | (33%) | 14 | (50%) | |
| 8 | 3 | (10%) | 4 | (14%) | |
| 9 | 0 | (0%) | 6 | (21%) | |

Table 3. Correlations between serum and urine markers. All correlations are rank based “Spearman's rho”.

TABLE 3

| | Correlation coefficient | P value | n |
|---|---|---|---|
| Sarcosine | 0.19 | 0.34 | 28 |
| Dimethylglycine | 0.12 | 0.53 | 28 |
| Cysteine | 0.33 | 0.06 | 33 |
| Homocysteine | 0.13 | 0.48 | 34 |

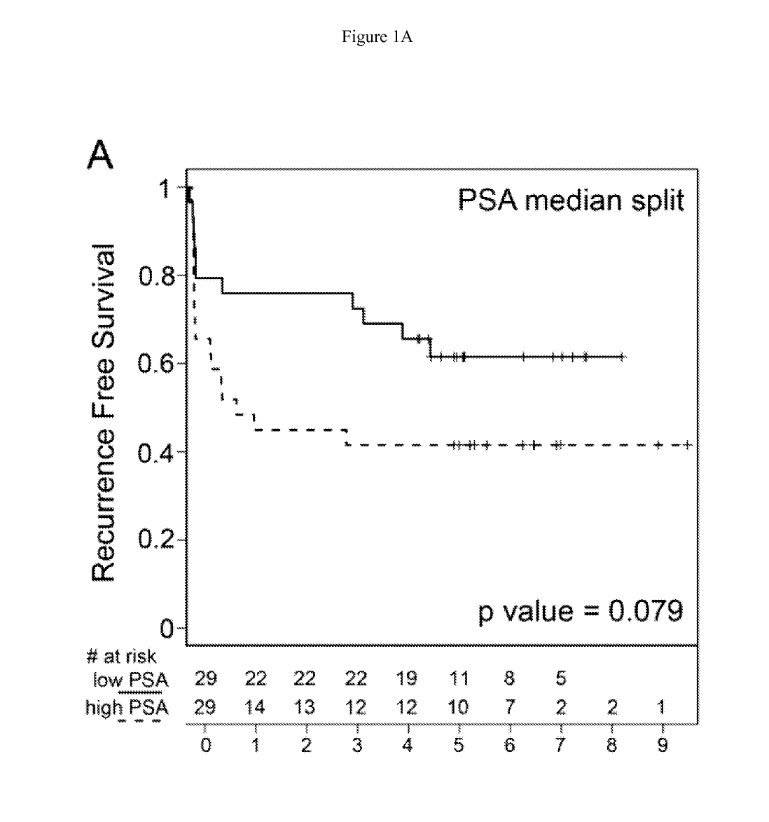

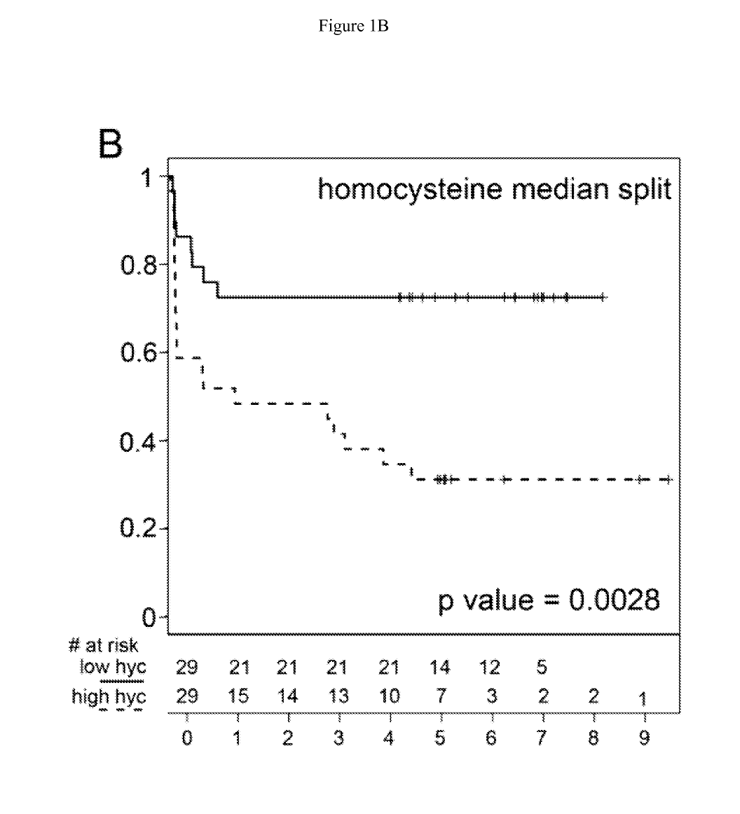

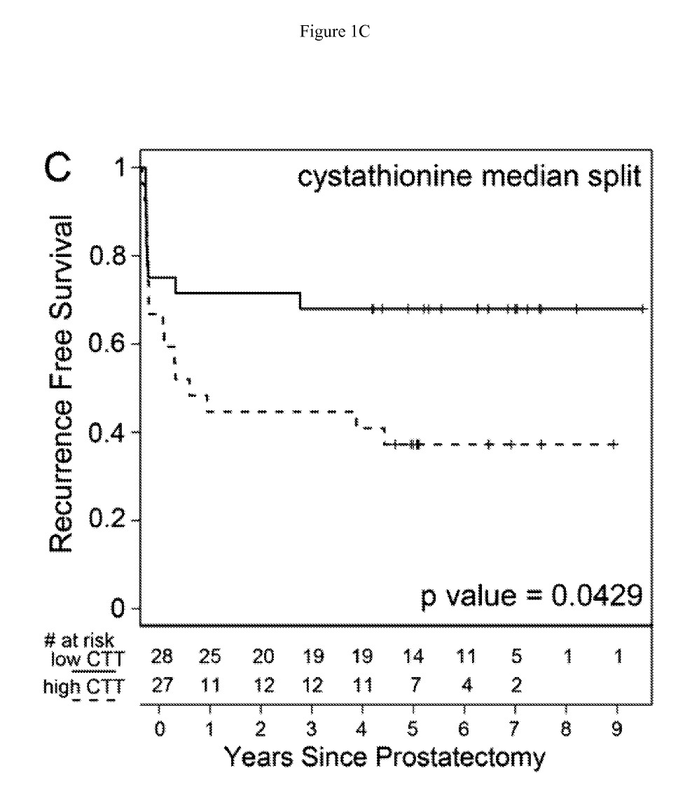

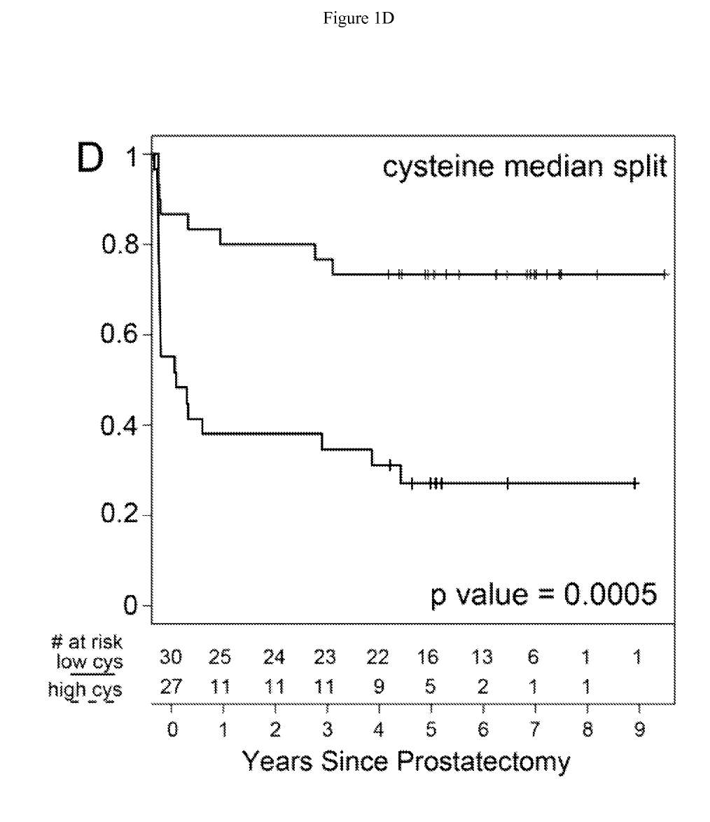

The relevance of these newly identified markers to patient recurrence status were illustrated in Kaplan-Meier plots for homocysteine, cystathionine, and cysteine as compared to pre-operative serum PSA levels, and time-to-recurrence (FIGS. 1A-1D). Each of the markers could separate rapidly recurrent from the recurrence-free progression. However, serum cysteine detection had the greatest discriminatory power in the two populations prior to prostatectomy.

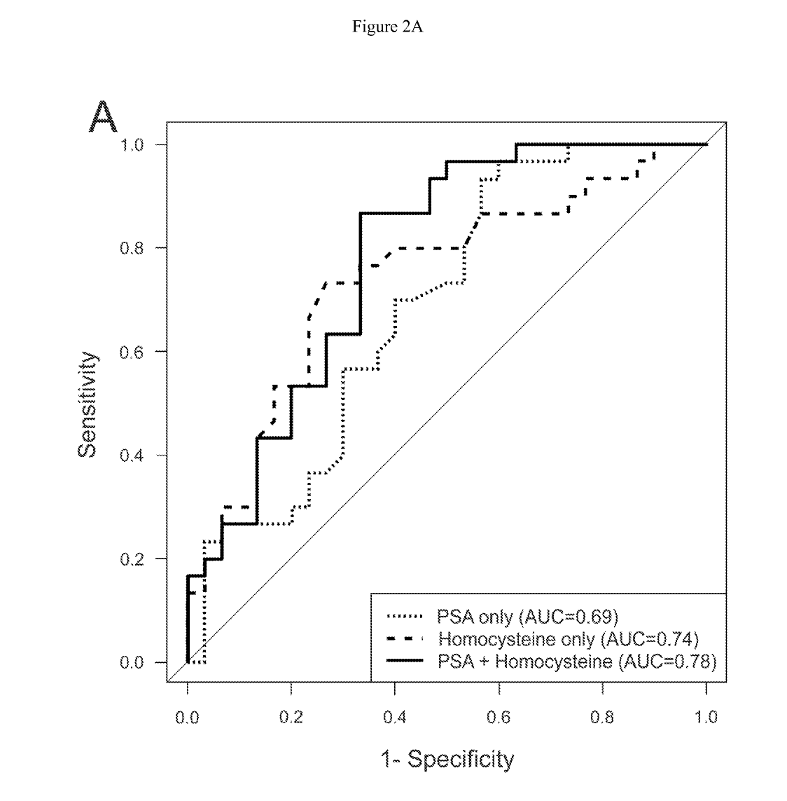

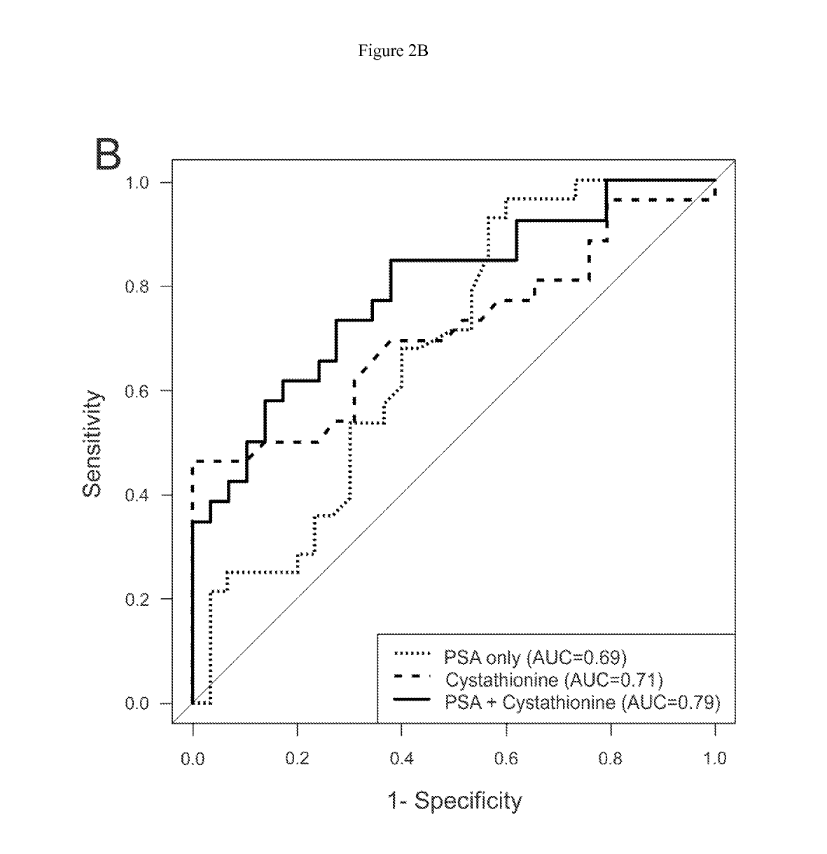

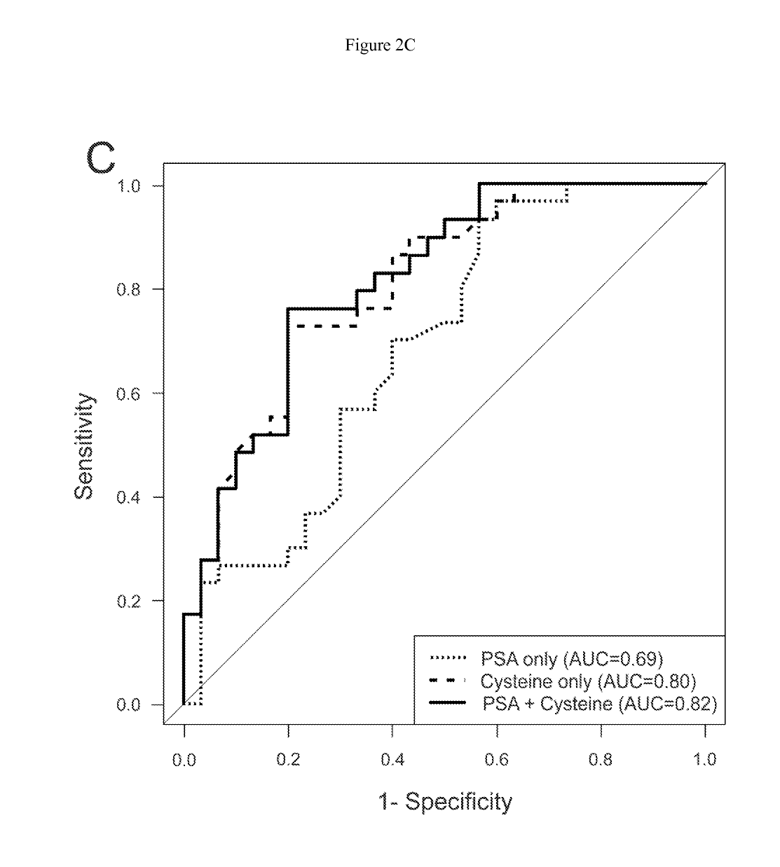

The clinical value of these methionine metabolites as biomarkers would be to significantly increase the ability to predict aggressive prostate cancer features and early biochemical recurrence over and above existent clinical variables including serum PSA, biopsy Gleason score, and clinical stage. We developed a multiple logistic regression model for the prediction of biochemical recurrence based on serum methionine metabolites and the pre-surgical predictor variables, serum PSA and biopsy Gleason grade. Since majority of patients in both cohorts had clinical stage T1c disease, this variable had little discriminative power and was dropped from the model. Serum cysteine, cystathionine, and homocysteine were the top three predictors for recurrence in 70% of the patients, so further analysis of methionine metabolites focused on these three metabolites. Correlations between cysteine and homocysteine were the highest among all pair-wise correlations (R²=0.65, p<0.01), and cysteine was also highly correlated with cystathionine (R²=0.39, p<0.01, Table 4). Addition of serum homocysteine provided the greatest improvement of the logistic regression models compared to the base model with PSA and biopsy Gleason (p=0.0007), followed by cysteine (p=0017), and cystathionine (p=0.0037). Correlation between cystathionine and homocysteine was moderate (R²=0.22, p=0.10). Based on multiple logistic regression models (Table 5), odds of recurrence increased 5.79 fold (95% CI: 1.65 to 20.29, p=0.006) when cysteine levels increased from 343 (lower quartile, henceforth Q1) to 436 (upper quartile, henceforth Q3). This logistic regression model did not find pre-surgical serum PSA levels to be significantly associated with recurrence status. In a separate model, cystathionine levels were significantly associated with recurrence status. Odds of recurrence were 2.44 (95% CI: 1.07 to 5.56, p=0.03) times higher when cystathionine levels were increased from 139 (Q1) to 200 (Q3). Serum PSA levels were marginally associated with recurrence in this model; the odds ratio was 2.94 (95% CI: 1.02 to 8.48, p=0.046) when PSA levels were increased from 4.7 (QI) to 8.5 (Q3). Homocysteine levels were also found to be associated with recurrence status. In all of these models biopsy Gleason grade was significantly associated with recurrence. To evaluate the additional utility of these three markers, the models including cysteine, cystathionine, or homocysteine in addition to serum PSA levels and biopsy Gleason grade were compared to a model utilizing PSA plus biopsy Gleason only. Clinical stage values did not contribute to the improvement of the models. Area under the ROC curves were similar (AUC=0.86) for the cysteine, cystathionine, and homocysteine when combined with the clinical variables and significantly superior to the clinical variables alone (AUC=0.81). The Integrated Discrimination Improvement (IDI) and Net Reclassification Improvement (NRI) supported the statistical significance of the improvement (Table 6). The benefit of these metabolites as combined with the standard PSA test is evident when PSA sensitivity and specificity were compared to a combined prediction of biochemical recurrence by the ROC in FIG. 2 following prostatectomy, using only serum PSA. The AUC with only serum markers were similar to the more comprehensive ones including biopsy results. There was a significant association between these markers and recurrence status; however the markers did not necessarily indicate usefulness in predicting recurrence-free survival.

Table 4. Correlations among serum markers. All correlations are rank based “Spearman's rho”, presented as correlation, p-value, and n.

TABLE 4

| | Dimethylglycine | Sarcosine | Cysteine | Cystathionine |
|---|---|---|---|---|
| Homo- | 0.28, 0.05 | 0.28, 0.04 | 0.65, <0.01 | 0.22, 0.10 |
| cysteine | n = 50 | n = 50 | n = 57 | n = 55 |
| Dimethyl- | | 0.35, 0.01 | 0.40, <0.01 | 0.16, 0.26 |
| glycine | | n = 50 | n = 50 | n = 48 |
| Sarcosine | | | 0.35, <0.01 | 0.08, 0.60 |
| | | | n = 50 | n = 48 |
| Cysteine | | | | 0.39, <0.01 |
| | | | | n = 54 |

Table 5: Logistic regression models.

TABLE 5

| | | | | |
|---|---|---|---|---|
| | Comparison | | | P |
| Variable | Q3:Q1 | Odds | 95% Confidence Int. | value |

SERUM HOMOCYSTEINE MODEL

| | | | | |
|---|---|---|---|---|
| Pre-surgery PSA | 8.5:4.7 | 2.39 | (0.90, 6.33) | 0.080 |
| Biopsy GS | 7:6 | 4.29 | (1.59, 11.56) | 0.004 |
| Serum homocysteine | 12.5:8.6 | 4.74 | (1.61, 13.90) | 0.005 |

SERUM CYSTATHIONINE MODEL

| | | | | |
|---|---|---|---|---|
| Pre-surgery PSA | 8.5:4.7 | 2.94 | (1.02, 8.48) | 0.046 |
| Biopsy GS | 7:6 | 2.80 | (1.24, 6.28) | 0.013 |
| Serum cystathionine | 200:139 | 2.44 | (1.07, 5.56) | 0.033 |

SERUM CYSTEINE MODEL

| | | | | |
|---|---|---|---|---|
| Pre-surgery PSA | 8.5:4.7 | 1.82 | (0.66, 4.96) | 0.245 |
| Biopsy GS | 7:6 | 2.51 | (1.19, 5.31) | 0.015 |
| Serum cysteine | 436:343 | 5.79 | (1.65, 20.29) | 0.006 |

Table 6: The Integrated Discrimination Improvement (IDI) and Net Reclassification Improvement (NRI) were summarized below, supporting the statistical significance of the improvement.

TABLE 6

| | | | P- | | | P- |
|---|---|---|---|---|---|---|
| | IDI | 95% CI | value | NRI | 95% CI | value |
| Homocysteine | 0.14 | 0.05-0.24 | 0.003 | 1.03 | 0.52-1.55 | <0.001 |
| Cystathionine | 0.12 | 0.004-0.20 | 0.003 | 0.81 | 0.28-1.34 | 0.003 |
| Cysteine | 0.14 | 0.04-0.23 | 0.005 | 0.64 | 0.13-1.16 | 0.015 |

To define the efficacy of the markers in predicting recurrence-free survival, Cox proportional hazard regression models were fit showing that cysteine, cystathionine, and homocysteine were each independent predictors of recurrence-free survival when adjusting for pre-operative serum PSA and biopsy Gleason score (Table 7). Specifically, serum cysteine, cystathionine, and homocysteine values increased (p<0.001, p=0.014, p<0.001, respectively) with increased risk of recurrence on multivariable analysis with adjustment for both serum PSA and biopsy Gleason score.

Table 7: Cox regression models

TABLE 7

| | | | | |
|---|---|---|---|---|
| | Comparison | | 95% | |
| Variable | Q3:Q1 | Hazard | Confidence Int. | P value |

SERUM HOMOCYSTEINE MODEL

| | | | | |
|---|---|---|---|---|
| Pre-surgery PSA | 8.5:4.7 | 2.34 | (1.27, 4.32) | 0.007 |
| Biopsy GS | 7:6 | 2.01 | (1.44, 2.79) | <0.001 |
| Serum homocysteine | 12.5:8.6 | 2.43 | (1.48, 4.01) | <0.001 |

SERUM CYSTATHIONINE MODEL

| | | | | |
|---|---|---|---|---|
| Pre-surgery PSA | 8.5:4.7 | 2.47 | (1.30, 4.70) | 0.006 |
| Biopsy GS | 7:6 | 1.64 | (1.21, 2.22) | 0.001 |
| Serum cystathionine | 200:139 | 1.69 | (1.11, 2.57) | 0.014 |

SERUM CYSTEINE MODEL

| | | | | |
|---|---|---|---|---|
| Pre-surgery PSA | 8.5:4.7 | 2.00 | (1.03, 3.86) | 0.039 |
| Biopsy GS | 7:6 | 1.71 | (1.24, 2.37) | 0.001 |
| Serum cysteine | 436:343 | 2.59 | (1.51, 4.43) | <0.001 |

### Example 4

The enzyme conversion step can be applied to other cysteine detection methods, assays, and systems to achieve significantly improved sensitivity and specificity. The enzyme-treated analytes in the serum or urine can be detected using various cysteine detection systems including, but limited to, HPLC, gas chromatography coupled mass spectroscopy (GC-MS), a nanorod-based assay (FIGS. 4 and 6-9), and a nanoelectronic device (FIG. 12). As such, the present invention provides a method of preparing a sample for an assay to determine cysteine level and a method of detecting a cysteine level in a sample from a subject.

For detection of cysteine in serum, 500 μl is minimally required. (1) Urinary creatine and albumin levels are needed to determine eligibility for the test. Elevated urinary creatine and albumin (>1.2 mg/dL and >8 mg/dL, respectively) would exclude the use of the cysteine assay for the subject. To enable efficient detection of cystathionine and homocysteine, the following will be added to each tube: serine, pyridoxal phosphate, cystathionine beta-synthase, and cystathionine gamma-lyase, and pH adjusted to 5.0. (2) This reaction is allowed to proceed for 1 to 12 hours at 32° C. (3) The reaction is filtered through a 3000 Da molecular weight spin column at 10,000 rpm for 30 min. The filtered reaction is prepared by a ten-fold dilution with phosphate buffered saline or water. (4a) The prepared sample will be analyzed by HPLC. Example settings of the HPLC analysis are 1 ml. Example settings of the HPLC analysis include the use of C18 reverse-phase column and detected by absorption, fluorescence of radio-labeling. (4b) Alternatively, the filtered reaction (i.e., the prepared sample) will be analyzed by gas chromatography coupled mass spectroscopy (GC-MS). As HPLC and GC-MS are well-known techniques routinely used by one of ordinary skill in the art, one of ordinary skill in the art would have known how to tailor the HPLC or GC-MS settings according to the specific properties of samples, equipment, and analysis purpose (Steele et al., Anal Biochem. (2012) 429:45-52; Buckpitt et al., Anal Biochem. (1977) 83:168-77; Hartleb et al., Biomed Sci Appl. (2001) 764:409-43; Stabler et al., Anal Biochem. (1987) 162:185-96; Ubbink et al., Clin Chem. (1999) 45:670-5).

For detection of cysteine in urine, 500 μl is required. (1) Urinary creatine and albumin levels are needed to determine eligibility for the test. Elevated urinary creatine and albumin (>1.2 mg/dL and >8 mg/dL, respectively) would exclude the use of the cysteine assay for the subject. To enable efficient detection of cystathionine and homocysteine, the following will be added to each tube following addition of serine, pyridoxal phosphate, cystathionine beta-synthase, and cystathionine gamma-lyase, and pH adjusted to 5.0. (2) This reaction is allowed to proceed for 1 to 12 hours at 32° C. (3) The reaction is filtered through a 3000 Da molecular weight spin column at 10,000 rpm for 30 min. The filtered reaction is prepared by a ten-fold dilution with phosphate buffered saline or water. (4a) The prepared sample will be analyzed by HPLC. Example settings of the HPLC analysis are 1 ml.

Example settings of the HPLC analysis include the use of C18 reverse-phase column and detected by absorption, fluorescence of radio-labeling. (4b) Alternatively, the filtered reaction (i.e., the prepared sample) will be analyzed by gas chromatography coupled mass spectroscopy (GC-MS). As HPLC and GC-MS are well-known techniques routinely used by one of ordinary skill in the art, one of ordinary skill in the art would have known how to tailor the HPLC or GC-MS settings according to the specific properties of samples, equipment, and analysis purpose (Steele et al., Anal Biochem. (2012) 429:45-52; Buckpitt et al., Anal Biochem. (1977) 83:168-77; Hartleb et al., Biomed Sci Appl. (2001) 764:409-43); Stabler et al., Anal Biochem. (1987) 162:185-96; Ubbink et al., Clin Chem. (1999) 45:670-5).

### Example 4

### HPLC with Postcolumn Fluorimetric Detection

Prior to HPLC analysis, free cysteine is buffer-exchanged into 0.1% formic acid and reduced with TCEP (Tris(2-carboxyethyl)phosphine) at 37° C. for two to three hours; the final concentration of TCEP was 20 mM in 100 μL 0.1% formic acid. The reduction released cysteine previously adducted on the protein. The mixture is then heated for ten min at 55° C. in a heat block. After heating, 95 μL mobile phase buffer A were added, and 10 μL were injected and analyzed by RP HPLC. Chromatographic separation can be performed on an HPLC system, equipped with a Zorbax C18, 5 μm particle size, 2.1 mm×150 mm column (Agilent, Santa Clara, Calif., USA). Separation can be achieved using a gradient mobile phase consisting of 0.1% TFA (v/v) in water (solvent A) and 90% acetonitrile, 0.1% TFA, and 9.9% water (v/v, solvent B); UV detection was achieved at 215 nm. The column was equilibrated at 37% mobile phase B for 18 min prior to running samples. Gradient conditions were: 0-10 min, 37% B; 10-48 min, 37-43% B; 48-58 min, 43% B; 58-65 min, 43-90% B; 65-75 min, 90% B and return to 37% B in 1 min. Flow rate is 0.2 mL/min, injection amount was 12 μg and the column temperature was maintained at 60° C. Total run time was 76 min and the post-delay time for reconditioning the column with 37% B was 18 min. Derivatized standard mixtures is allowed to cool at room temperature and 95 μL mobile phase A were added to each standard. A volume of 10 μL of each standard was injected on the HPLC. To make a standard curve The final amounts of derivatized 1-cysteine standard injected were 30 pg, 60 pg, 120 pg, 240 pg, 360 pg, 480 pg, 1200 pg, 1800 pg, and 2400 pg. The amount of each thiol from adducted species is expressed as nmol adduct/nmol protein.

### Example 5

### GC-MS Method

The steps before GC-MS include the addition of deuterated internal standards first, addition of the reductant dithiothreitol and NaOH in a second pipetting, heating at 40° C. for 30 min, fractionation of sample on a disposable anion-exchange column, drying, and derivatization with N-methyl-N-(tertbutyldimethylsilyl)trifluoroacetamide. The tert-butyldimethylsilyl derivatives are separated and quantified by capillary GC-MS in the selected-ion monitoring mode. The samples are analyzed on a Durabond DB-I fused silica capillary column (30 m×0.25 mm i.d., 0.25 Mm film thickness) and 59928 gas chromatograph-mass spectrometer equipped with a falling needle injector. Quantitation is based on the ratio of the areas of the base peak ion 420.2 for homocysteine, 320.2 for methionine, and 406.2 for cysteine, each of which elutes at a different time, to the areas of the base peak ions of 424.2, 323.2, and 408.2 for the derivatives of their respective stable isotope internal standards.

### Example 6

Prostate cancer is often an over-treated disease due to our inability to distinguish its indolent and aggressive manifestations. Aggressive prostate cancer is characterized by its ability to survive and proliferate at hypoxic and nutrient deficient conditions in local or distant sites of metastasis. Metastatic progression is associated with recurrent disease following local therapeutic intervention. Accordingly, it has been demonstrated that patients with recurrent prostate cancer following prostatectomy have elevated sulfur containing amino acids, homocysteine (p value=0.003), cystathionine (p value=0.04), and cysteine (p value<0.0001), detectable in serum. (Normal serum values are: cysteine 203-369 μM, homocysteine 5.4-13.9 μM, and cystathionine 44-342 μM.) Gleason grade and clinical stage of the recurrent and non-recurrent patients were not statistically significant (n=58). A primary lack of diagnostic use of these factors lies in its high cost and difficulty for high throughput analysis. A unique technique as described herein is developed to convert and detect the three biomarkers as a single end product. Since the levels of all these individual amino acids are part of single metabolic pathway and are elevated in recurrent disease, the conversion of homocysteine and cystathionine to cysteine can be achieved by ex vivo incubation with cystathionine beta syntheses (CBS) and cystathionine gamma lyase (CGL), respectively. Helicobacter pylori CBS and CGL had been cloned and expressed in E. coli after codon optimization, and purified using N-terminal cellulose binding domain (Clostridium thermocellum). More than 80% enzyme conversion of homocysteine and cystathionine was achieved. High performance liquid chromatography and spectroscopic techniques were used to determine the enzyme activity. Following enzymatic conversion, samples were filtered and incubated with polymer-coated gold-nanorods and Cu²⁺ at room temperature. Subsequent absorption spectrum analysis demonstrated a sharp red-shift of longitudinal surface plasmon peaks in a concentration linearly dependent on the concentration of free cysteine (0-100 μM; R²>0.93). The plasmon peak shift took place due to end to end joining of gold-nanorods resulting formation of linear chains, validated by transmission electron microscopic visualization. The translation of the method of these findings was determined by measuring cysteine in mice xenografted with metastatic prostate tumors. Cysteine measurements were correlated to tumor progression by longitudinal monitoring of luciferase labeled tumor (ARcaPM) bioluminescent detection and followed by H&E. Finally, annotated serum samples of prostate cancer patients were analyzed retrospectively. The serum was collected prior to prostatectomy and outcomes were followed up to five years following prostatectomy. Biochemically recurrent and non-recurrent subjects were successfully distinguished using this modified gold nanorod technique. The advantages of this system over the existing methods include: its adaptability to high throughput analysis, economical, minimal sample requirement (˜200 μl) and reduced technical complexity.

Quantitative detection methods involving modification by a radioactive treasure technique or fluorophore/chromophore conjugation followed by GC-MS or HPLC, are tedious, low throughput and expensive. Therefore, less expensive and efficient methods are needed for determination of blood and urine cysteine concentrations. Gold nanoparticles have very high affinity for free —HS group. Henceforth, gold particles became a potent tool for cysteine concentration determination.

Since all surfaces of gold nanoparticles have similar affinity for cysteine, so far any linear relationship had not been established between cysteine concentration and particle end to end joining. Therefore, in one embodiment, it is desirable to use nanorods with available gold tips only at the ends. A hybrid nanorod can be ideal for that, since there is nothing like that have been commercially available till now, the inventors used longitudinal surface nonreactive polymer protected gold rods that leave exposed gold tips only available on the ends. Polymers increase the stability of the rods in the water. End to end joining of particles increases the overall length of nanorods, leading to a red shift of characteristic plasmon peak.

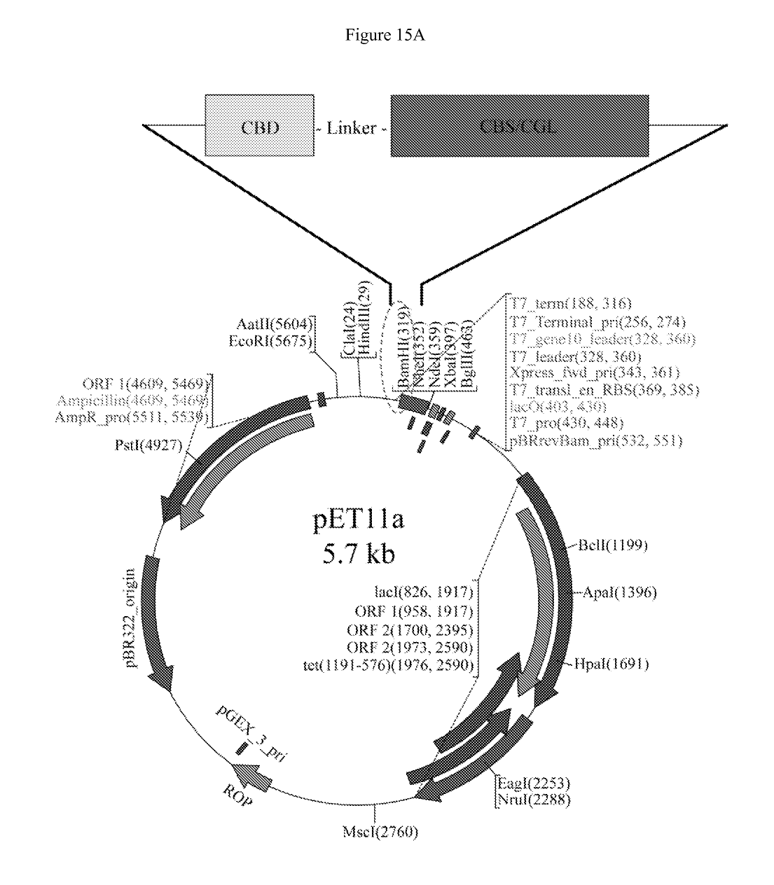

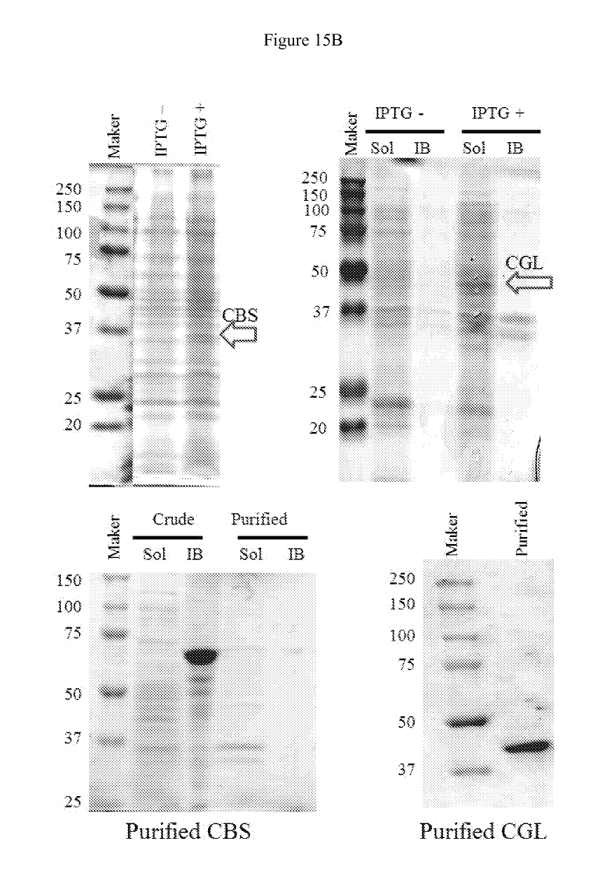

CBS and CGL are cloned, expressed and purified (FIGS. 15A-15B). Sequences were optimized for E. coli expression. Cellulose binding domain (CBD) protein sequence was taken from Clostridium thermocellum. CBS and CGL protein sequences were taken from Helicobacter pylori. Linker is 6 amino acids (GAGAGA). Protein induction was given at 18° C. for 24 h using 0.75 mM IPTG. CBS and CGL were purified in cellulose beads bound condition. About 40±5 μg of proteins were found to be bound with 1 mg of cellulose beads.

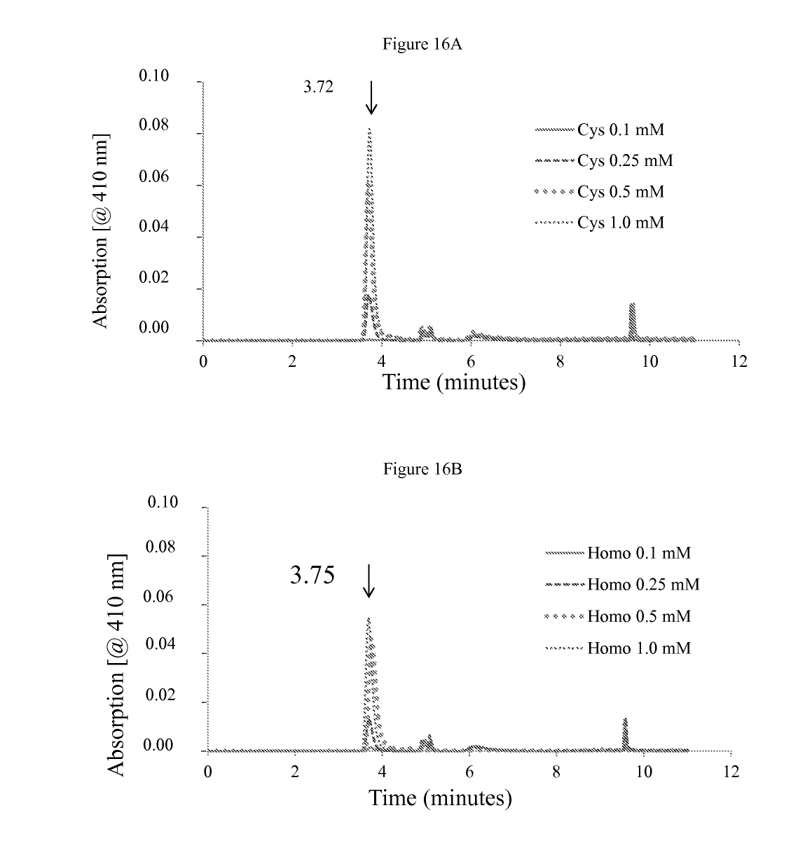

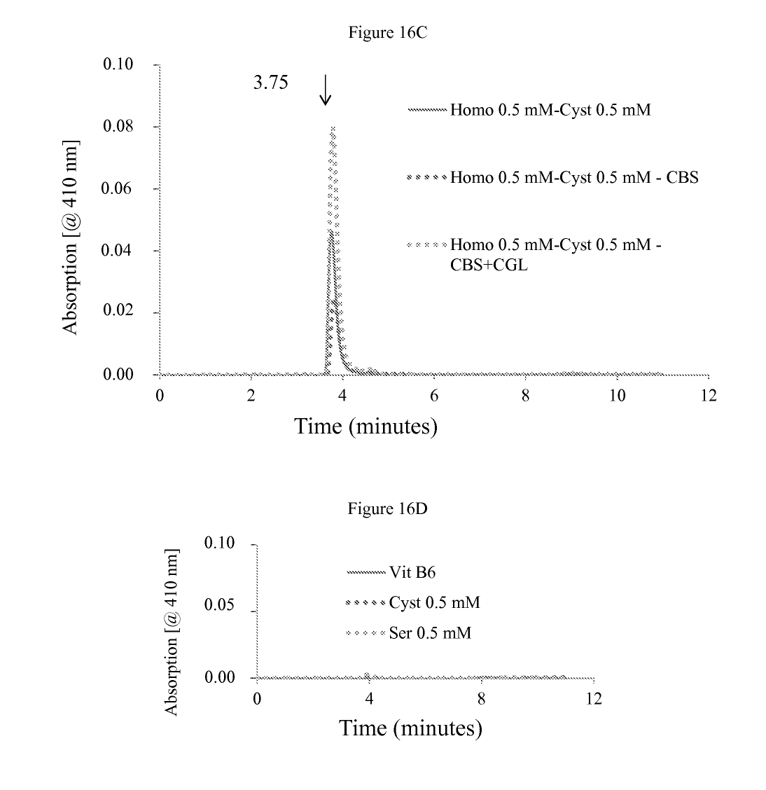

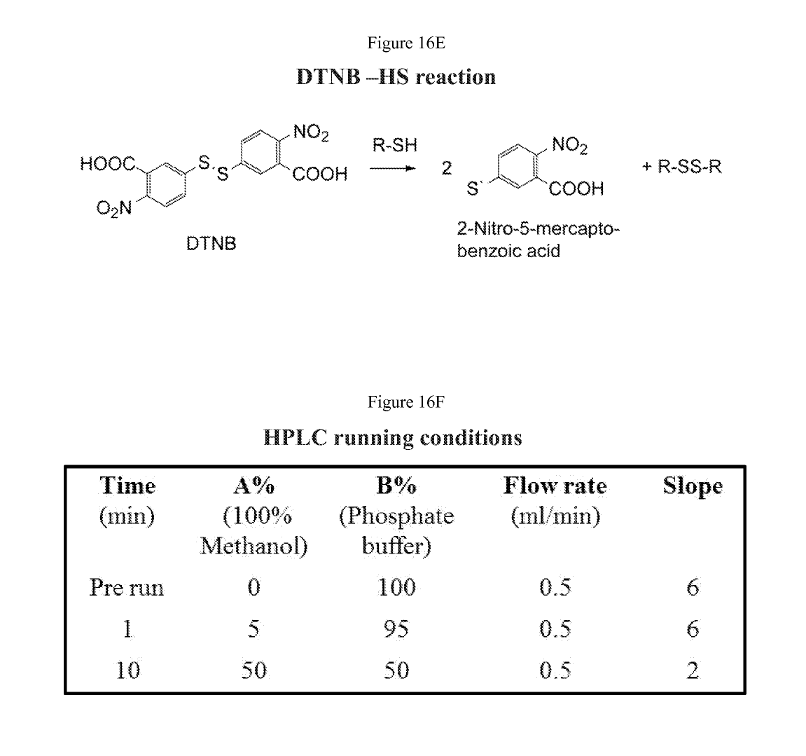

CBS and CGL activities were determined by HPLC (FIGS. 16A-16F). DTNB generates equal molar amount of TNB upon reaction with a sulfur group. TNB has specific absorption at 410 nm. The amount of TNB formation is an indirect way to quantify the amount of reactive sulfur groups present in the system.

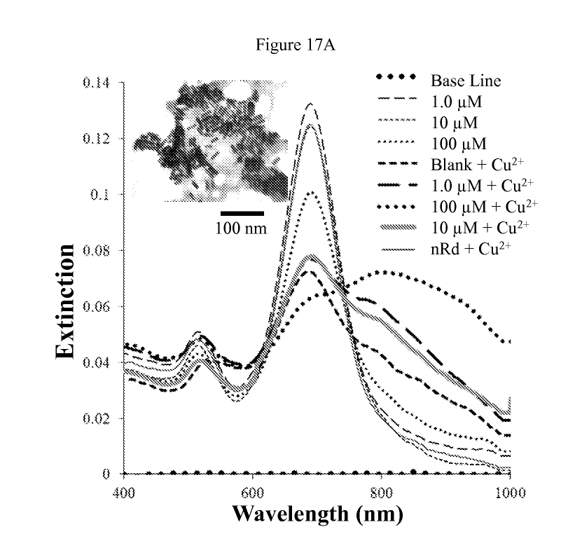

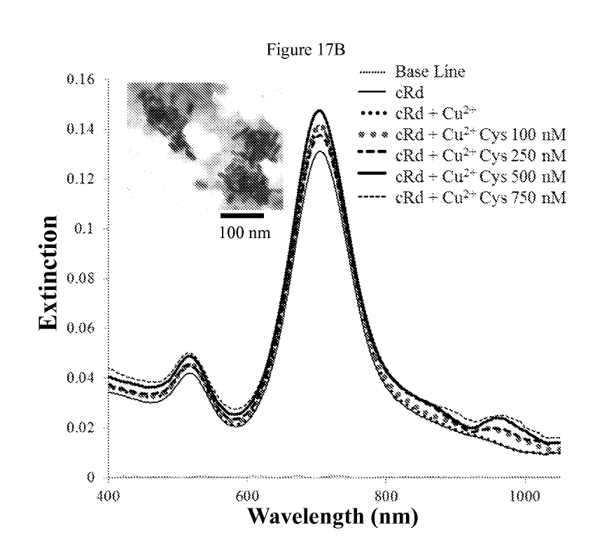

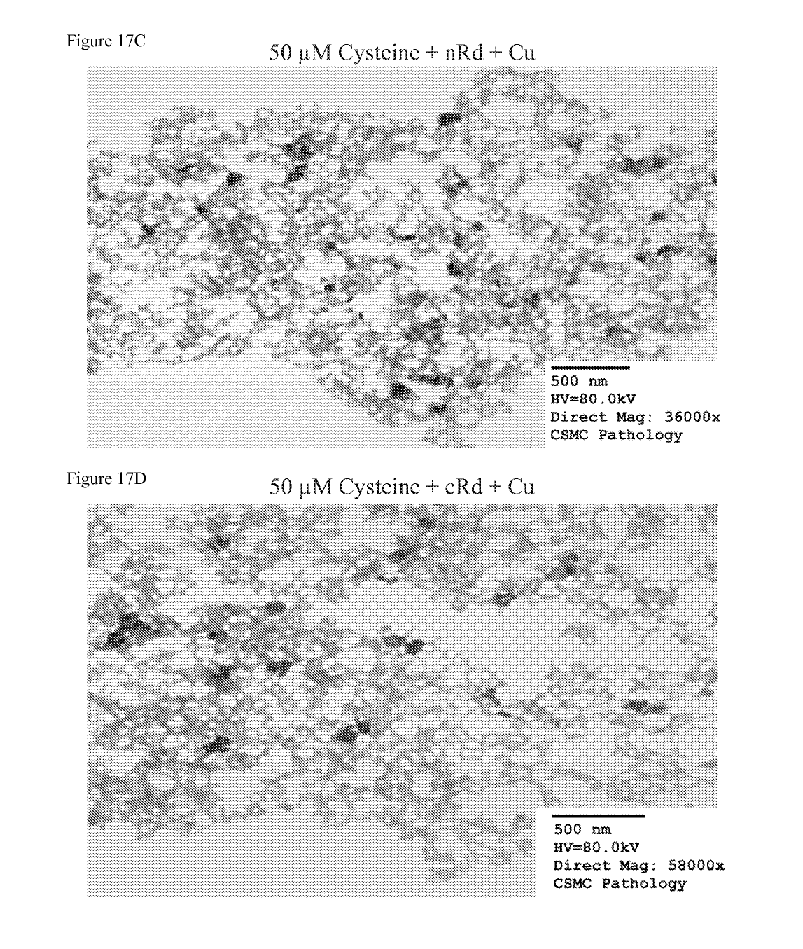

Naked and CTAB protected gold nanorods were used for cysteine titration (FIGS. 17A-17D). Naked gold nanorods (nRd) have equal affinity for cysteine or free sulfur group one all surfaces, therefore these particles end up with formation of big agglomerations. CTAB is not a very good surface protector. It has almost equal affinity on all surfaces of gold nanoparticles. Moreover it is not a very stable surface protector. Henceforth, CTAB protected particles (cRd) also form small agglomerations upon reaction with cysteine. This agglomeration formation is characterized by an increase of extinction at 800-1000 nm with a decrease of longitudinal peak intensity, and not by plasmonic peak shift.

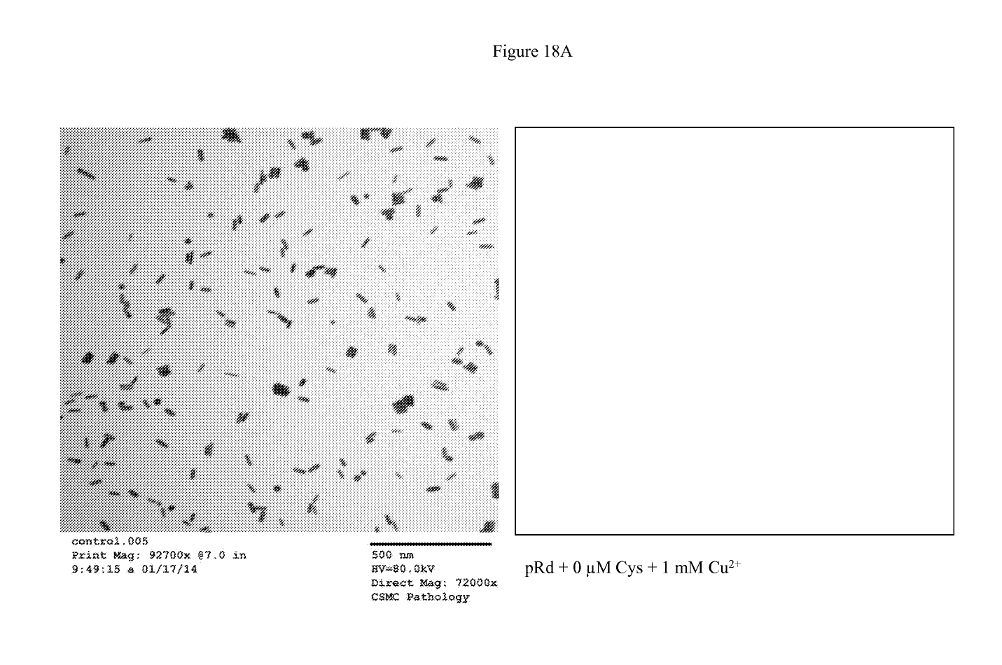

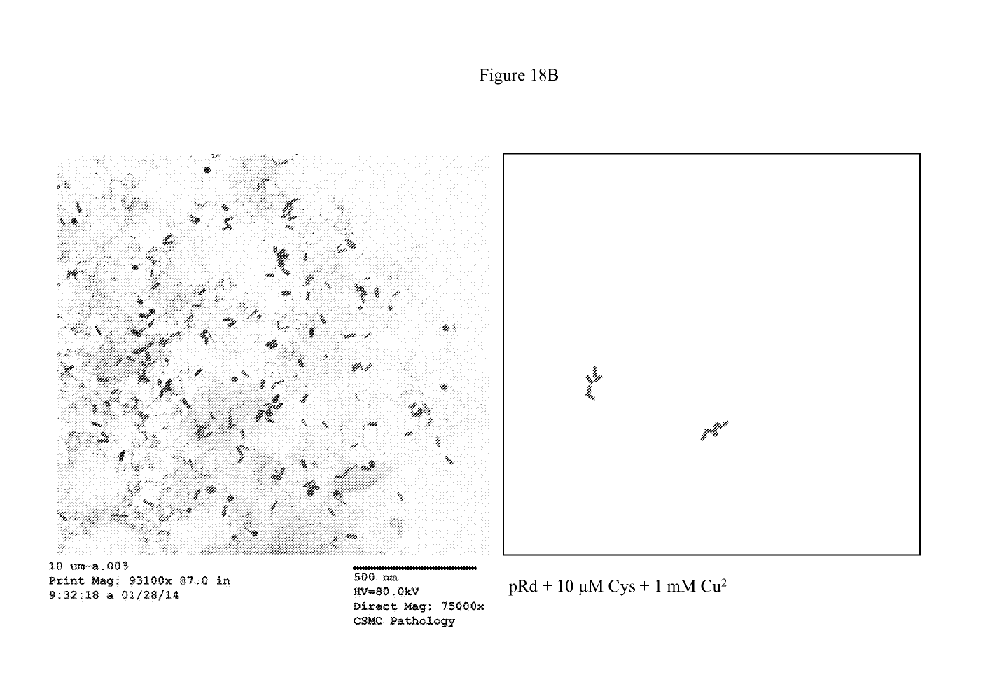

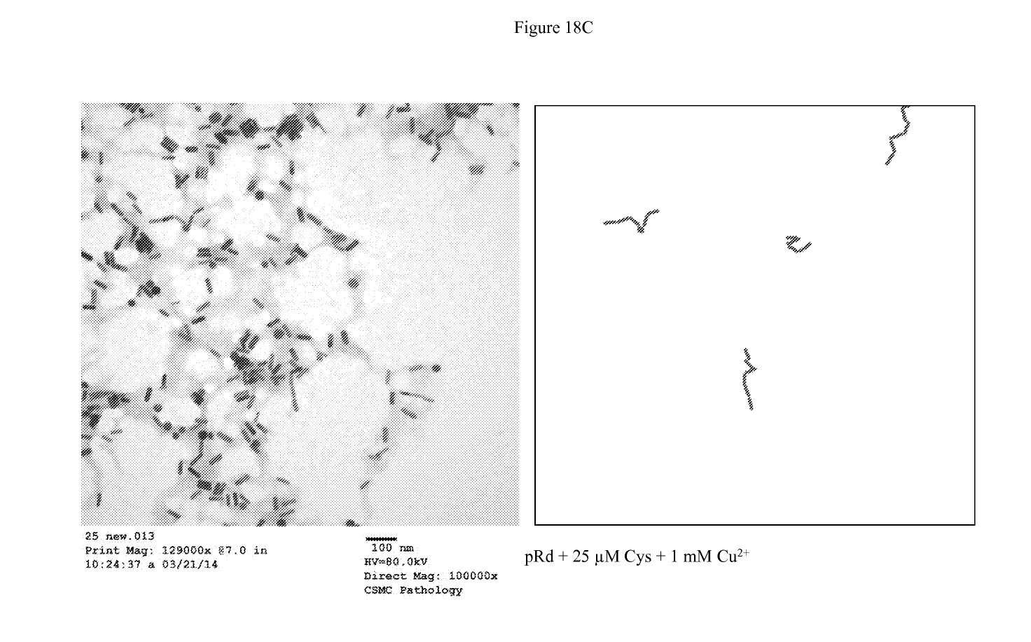

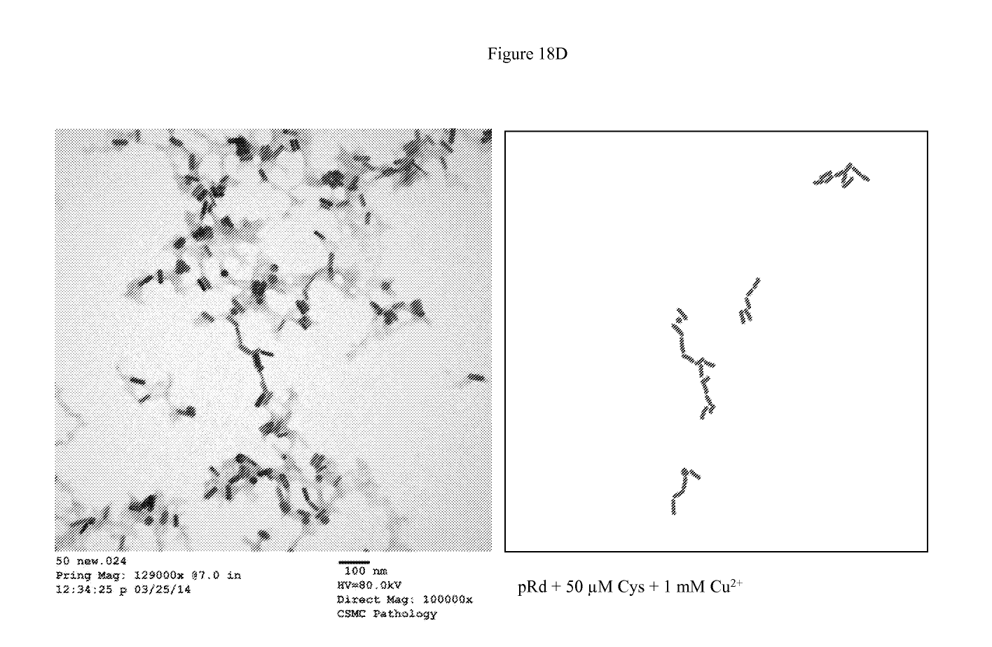

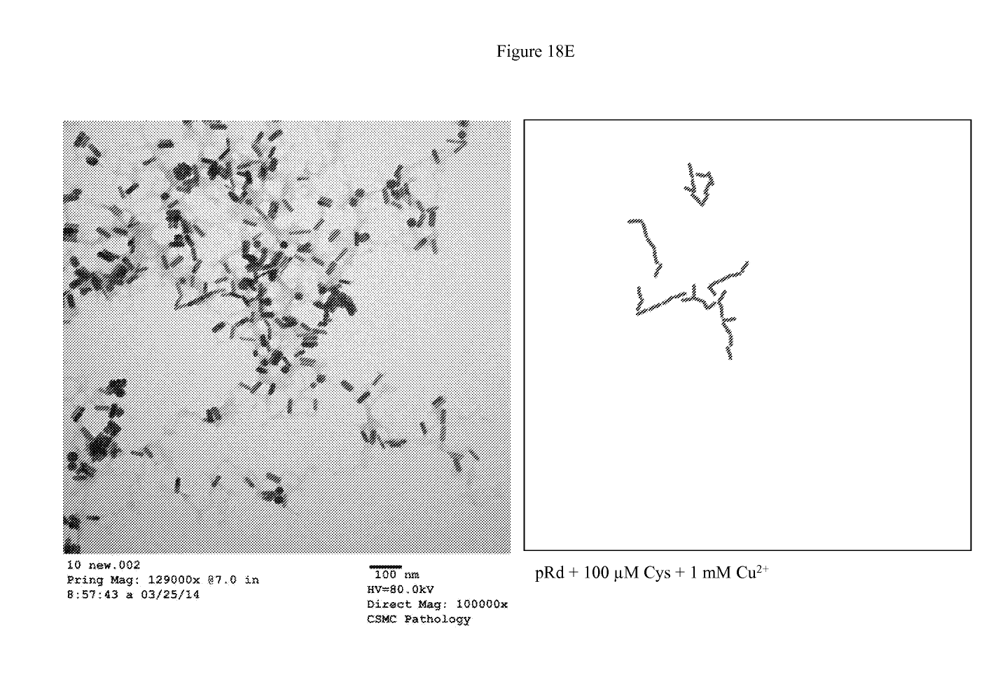

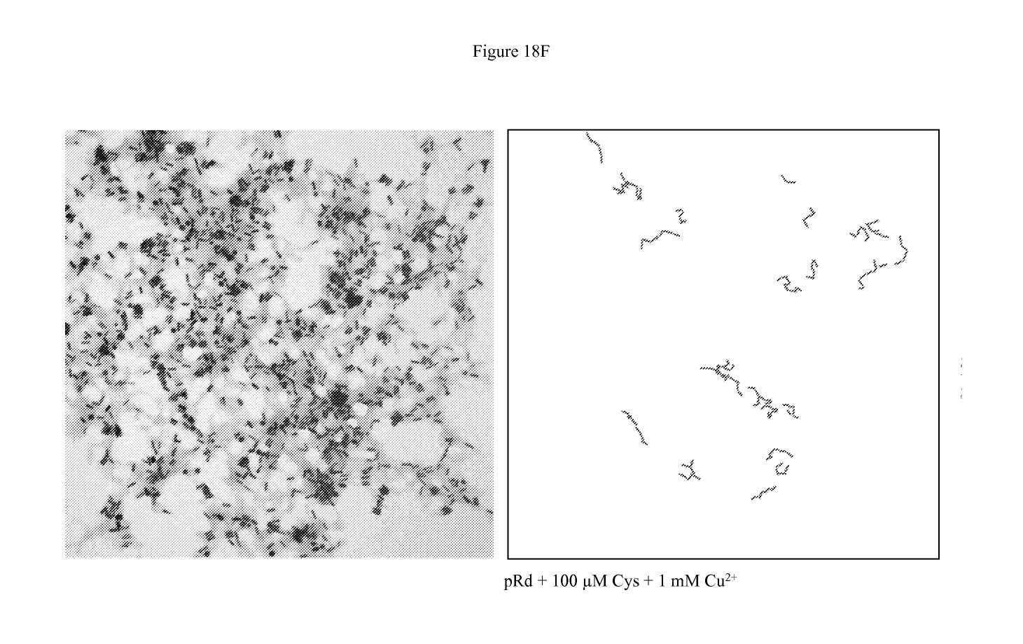

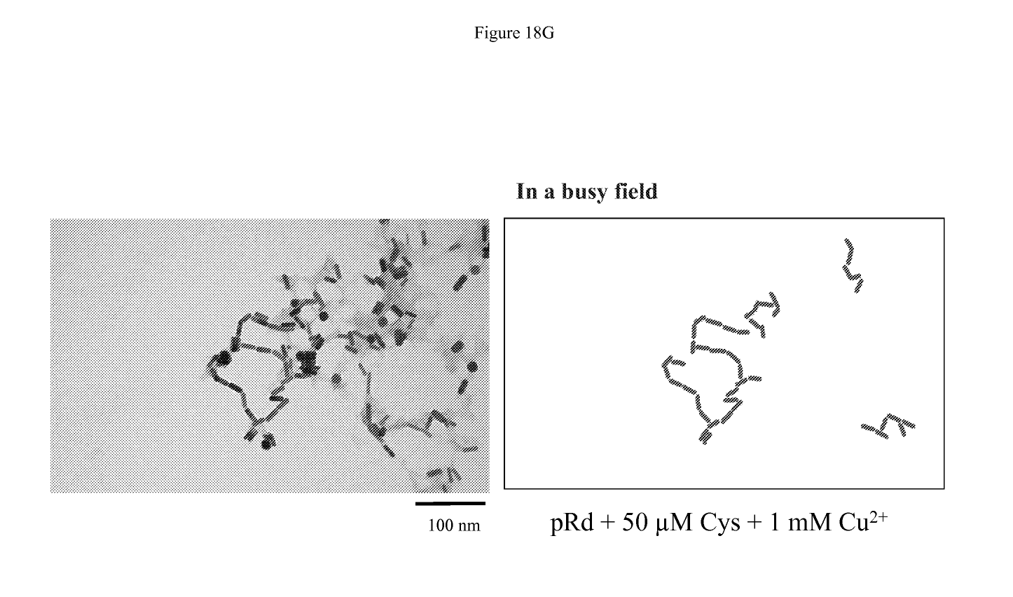

pRd reaction with cysteine results in formation of linearly joined long chain nanopolymer (FIGS. 18A-18G). Polymer protected gold nanorods formed nanochains upon reaction with cysteine. The chain formation is cysteine concentration dependent. The red tracings demonstrate nanopolymer chains observed in the respective TEM filed.

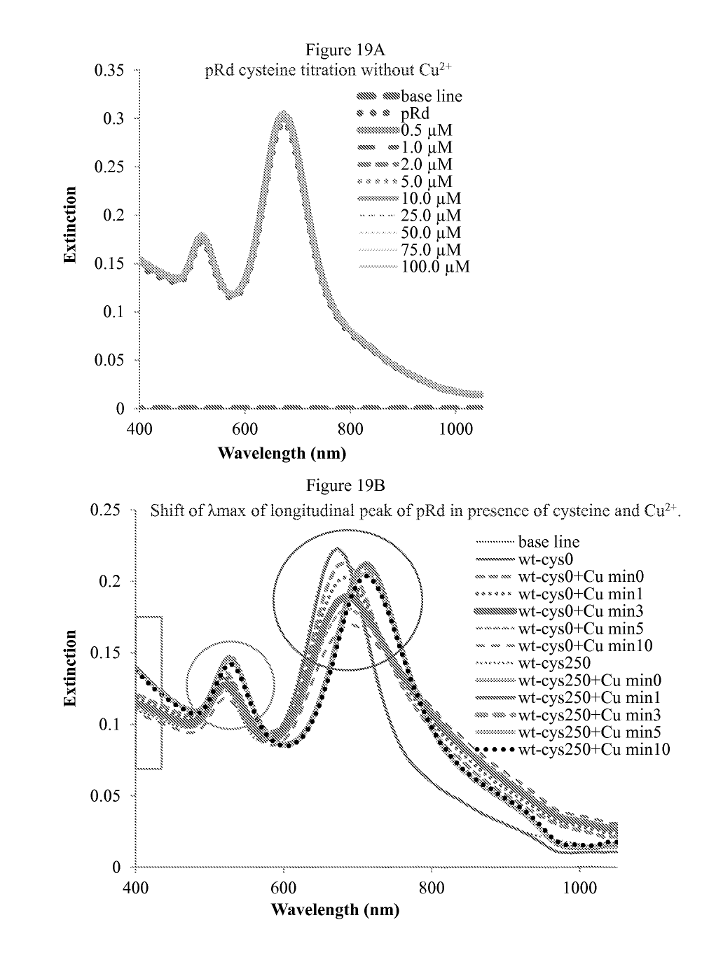

Plasmonic properties of pRd changed upon reaction with cysteine (FIGS. 19A-19B). Polymer protected gold nanoparticles showed a red shift of longitudinal plasmon peak. This peak shift is copper (II) ion dependent. pRd upon reaction with cysteine showed one major plasmon peak shift (red big circle) due to end to end joining of nanoparticles, and two minor shifts: one shift at transverse peak (blue small circle) due to vibration of nanoparticles at the joined ends resulting in the formation of bigger hydrodynamic structures, and the other shift as an increase in extinction at around 400 nm (green box) due to increased turbidity of the system.

Shift of pRd longitudinal peak due to cysteine titration in presence of Cu²⁺ is concentration dependent and time independent (FIG. 7). Plasmonic shift due to cysteine reaction with pRd was characterized: the longitudinal peak shift due to reaction with cysteine is linearly related to cysteine concentration; in the presence of copper (II) ion, a single spontaneous time independent red shift of longitudinal peak took place (FIGS. 6A-6B).

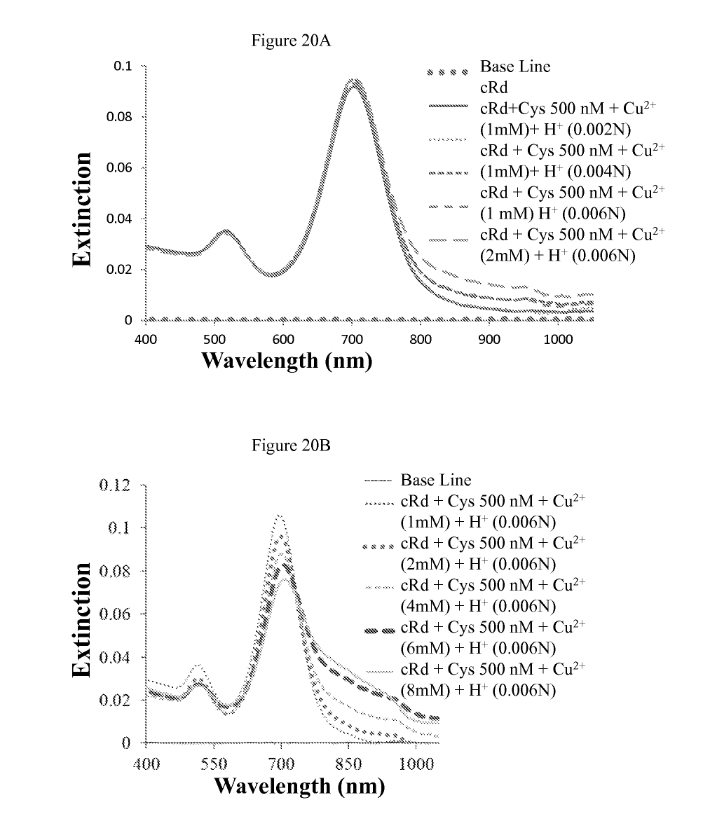

Effects of acid and Cu²⁺ on cysteine induced reassembly of cRd were investigated. Both acid and Cu²⁺ leaded to an increase in the intensity of 960 nm peak; but no peak shift was observed, suggesting formation of big aggregates and not nanochains (FIGS. 20A-20B).

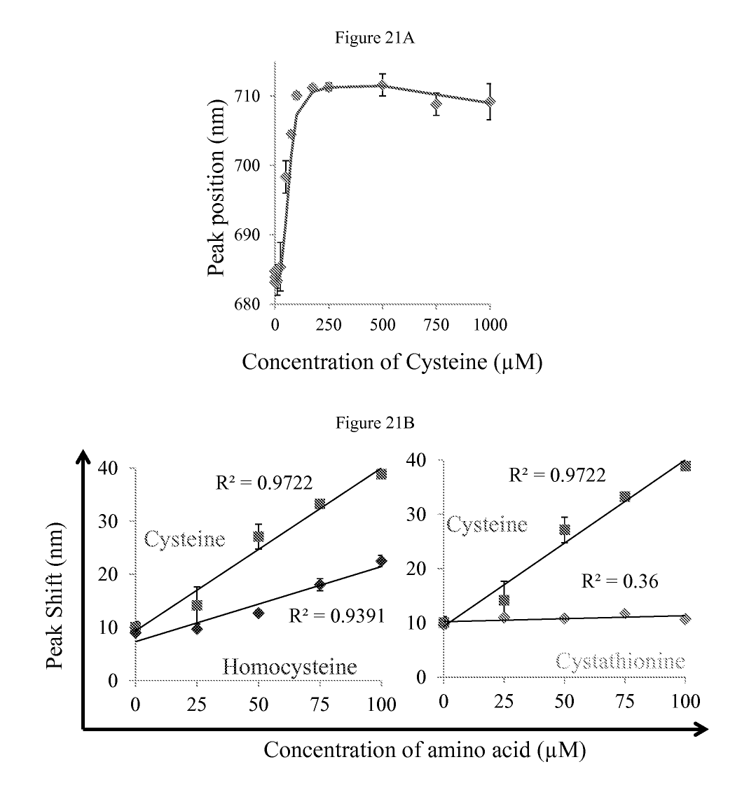

pRd based sulfur amino acid titration standard curve was obtained, for example, according to cysteine concentration dependent longitudinal peak shift of pRd (FIGS. 21A-21B). pRd of 30:10 nm showed peak shift in a linear manner till 100 μM, and a plateau in peak shift was observed beyond that concentration. Peak shifts of cysteine, homocysteine and cystathionine were compared. Cysteine due to the maximum affinity for gold particles showed the maximum peak shift, followed by homocysteine. Cystathionine does not have any affinity for pRd and therefore did not show any peak shift.

Cysteine after spiking in the human serum was recovered. Different concentrations of purified cysteine were spiked in the serum and incubated for 30 mins at RT. After incubation, the serum was filtered using 3 kD filter and the effluent was used to estimate the cysteine concentrations using pRD (FIG. 9).

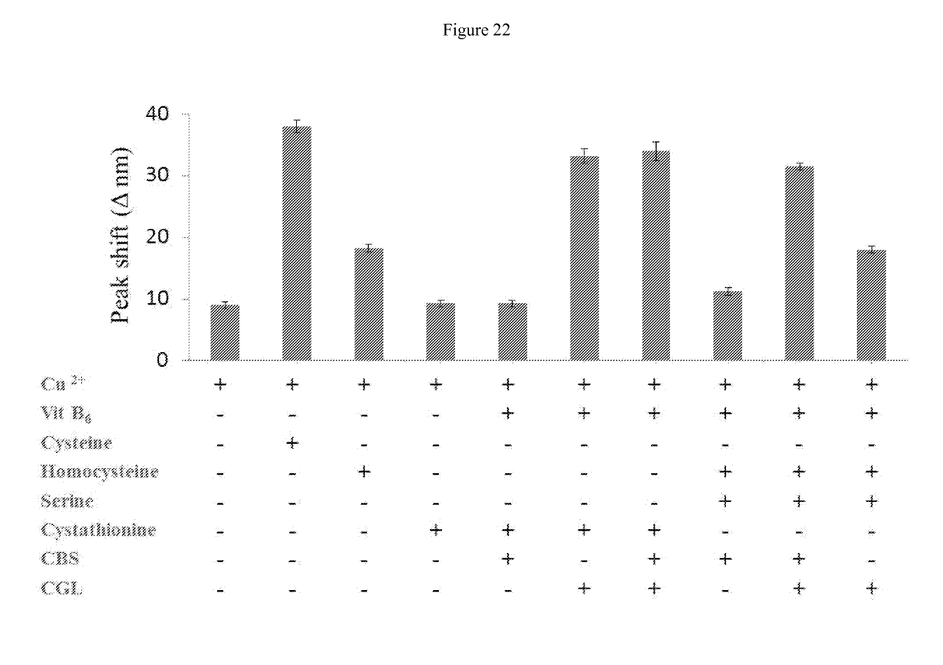

CBS and CGL activities were determined by plasmon shift assay (FIG. 22). All systems were incubated at room temperature for 6 hr, before incubation with pRd. Samples were incubated with pRd for 30 min at RT and then 1 mM CuCl₂ solution. Spectral readings were taken 5 minutes after addition of Cu²⁺.

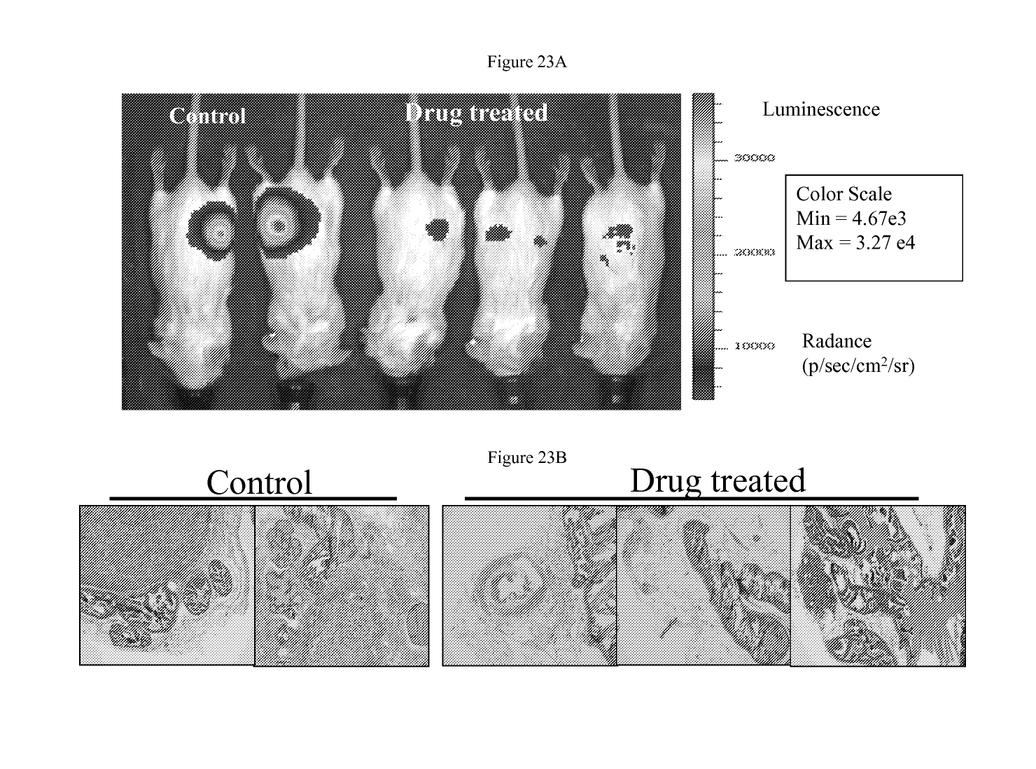

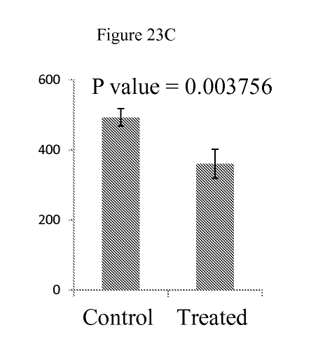

Serum cysteine concentrations and their prognostic value were evaluated in mice. Bioluminescence of ARcaPM grafted tumor was shown (FIG. 23A). Human prostate cancer cell line (luciferase labeled ARcaPM) cells were grafted in kidney capsule of SCID mice. The mice tumors were allowed to grow for 30 days in the presence or absence of drug. Tumor sizes from the live mice were measured after 30 days of incubation period. Hematoxylin and eosin (H&E) stained sections of grafted tumors from the mice were shown (FIG. 23B). Serum cysteine concentrations were measured from the mice using pRd (FIG. 23C). Serum cysteine concentrations of the control tumor-carrying mice are significantly higher than the drug treated mice.

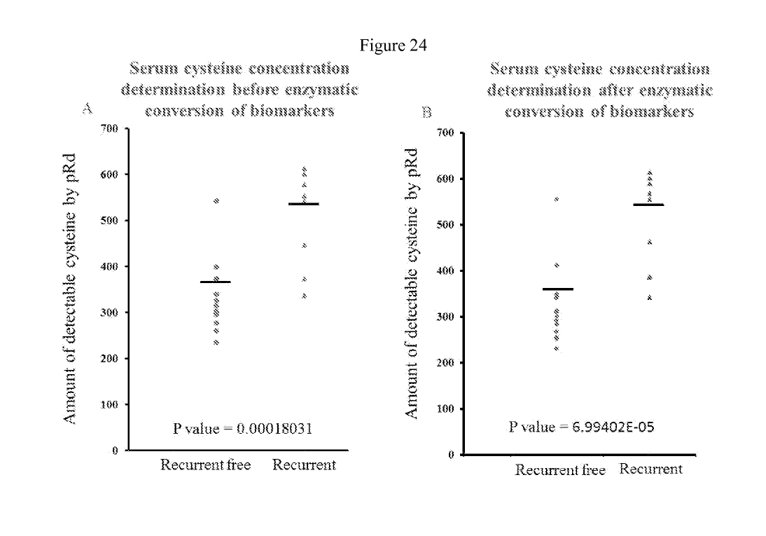

Serum cysteine levels in prostate cancer patients were detected using pRd before and after enzymatic conversion of the biomarkers. Serum cysteine concentrations were determined from recurrent and recurrent free prostate cancer patients before enzymatic conversion (FIG. 24A) and after enzymatic conversion (FIG. 24B). Cystathionine beta synthase and cystathionine gamma lyase convert homocysteine to cystathionine and cystathionine to cysteine respectively. Conversion and compression of these biomarkers allowed better distinguishing between recurrent and recurrent free prostate cancer groups.

The gold standard for estimating serum cysteine, homocysteine and cystathionine can be viewed as GC-MS using a radioactive treasure technique. This technique is tedious and expensive and costs around 600-800 USD per patient. HPLC is a relatively cheaper technique to estimate serum cysteine levels. For HPLC, conjugation of cysteine with some fluorophore or chromophore is also needed. Furthermore, HPLC is not very quantitative as well as it is not high throughput. Therefore, both GC-MS and HPLC were never used for clinical practice. The polymer protected nanoparticle based technique is cheap and high throughput, and a combination of this technique with enzymatic conversion of biomarker makes the technique a tool for clinical use.

### REFERENCES

1. Etzioni R, Penson D F, Legler J M, di Tommaso D, Boer R, et al. (2002) Overdiagnosis due to prostate-specific antigen screening: lessons from U.S. prostate cancer incidence trends. J Natl Cancer inst 94: 981-990.
2. Andriole G L, Crawford E D, Grubb R L, 3rd, Buys S S, Chia D, et al. (2009) Mortality results from a randomized prostate-cancer screening trial. N Engl J Med 360: 1310-1319.
3. Schroder F H, Hugosson J, Roobol M J, Tammela T L, Ciatto S, et al. (2009) Screening and prostate-cancer mortality in a randomized European study. N Engl J Med 360: 1320-1328.
4. Thompson I M, Pauler D K, Goodman P J, Tangen C M, Lucia M S, et al. (2004) Prevalence of prostate cancer among men with a prostate-specific antigen level< or =4.0 ng per milliliter. N Engl J Med 350: 2239-2246.
5. Thompson I M, Ankerst D P, Chi C, Lucia M S, Goodman P J, et al. (2005) Operating characteristics of prostate-specific antigen in men with an initial PSA level of 3.0 ng/ml or lower. Jama 294: 66-70.
6. Blum D L, Koyama T. M'Koma A E, Iturregui J M, Martinez-Ferrer M, et al. (2008) Chemokine markers predict biochemical recurrence of prostate cancer following prostatectomy. Clin Cancer Res 14: 7790-7797.
7. Thompson K E, Hernandez J, Canby-Hagino E D, Troyer D, Thompson I M (2005) Prognostic features in men who died of prostate cancer. J Urol 174: 553-556; discussion 556.
8. Sreekumar A, Poisson L M, Rajendiran T M, Khan A P, Cao Q, et al. (2009) Metabolomic profiles delineate potential role for sarcosine in prostate cancer progression. Nature 457: 910-914.
9. Luka Z, Mudd S H, Wagner C (2009) Glycine N-methyltransferase and regulation of S-adenosylmethionine levels. J Biol Chem 284: 22507-22511.
10. Levy H L, Coulombe I T, Benjamin R (1984) Massachusetts Metabolic Disorders Screening Program: III. Sarcosinemia. Pediatrics 74: 509-513.
11. Allen R H, Stabler S P, Lindenbaum J (1993) Serum betaine, N,N-dimethylglycine and N-methylglycine levels in patients with cobalamin and folate deficiency and related inborn errors of metabolism. Metabolism 42: 1448-1460.
12. Allen R H, Stabler S P, Savage D G, Lindenbaum J (1993) Elevation of 2-methylcitric acid I and II levels in serum, urine, and cerebrospinal fluid of patients with cobalamin deficiency. Metabolism 42: 978-988.
13. Stabler S P, Marcell P D, Podell E R, Allen R H, Savage D G, et al. (1988) Elevation of total homocysteine in the serum of patients with cobalamin or folate deficiency detected by capillary gas chromatography-mass spectrometry. J Clin Invest 81: 466-474.
14. Home D W, Patterson D (1988) Lactobacillus casei microbiological assay of folic acid derivatives in 96-well microtiter plates. Clinical Chemistry 34; 2357-2359.
15. Barr D B, Wilder L C, Caudill S P, Gonzalez A J, Needham L L, et al. (2005) Urinary creatinine concentrations in the U.S. population: implications for urinary biologic monitoring measurements. Environ Health Perspect 113: 192-200.
16. Pencina M J, Sr DAR, D'Agostino R B Jr (2008) Evaluating the added predictive ability of a new marker: from area under the ROC curve to reclassification and beyond. Stat Med 27: 157-172.
17. Grambsch P, Therneau T (1994) Proportional hazards tests and diagnostics based on weighted residuals. Biometrica 81: 515-526.
18. Baik H W, Russell R M (1999) Vitamin B12 deficiency in the elderly. Annu Rev Nutr 19: 357-377.
19. Roehl K A, Antenor J A, Catalona W J (2002) Serial biopsy results in prostate cancer screening study. J Urol 167: 2435-2439.
20. Capitanio U, Karakiewicz P I, Jeldres C, Briganti A, Gallina A, et al. (2009) The probability of Gleason score upgrading between biopsy and radical prostatectomy can be accurately predicted. Int J Urol 16: 526-529.
21. Beam C A, Gao W, Macias V, Liang W, Kajdacsy Balla A (2009) Sequential testing approach as an efficient and easier alternative for the validation of new predictive technologies in the clinic. J Clin Oncol 27: 1087-1090.
22. Finkelstein J D, Kyle W E, Martin J L, Pick A M (1975) Activation of cystathionine synthase by adenosylmethionine and adenosylethionine. Biochem Biophys Res Commun 66: 81-87.
23. Jentzmik F, Stephan C, Miller K, Schrader M, Erbersdobler A, et al. (2010) Sarcosine in urine after digital rectal examination fails as a marker in prostate cancer detection and identification of aggressive tumours. Eur Urol 58: 12-18; discussion 20-11.
24. Hewavitharana A K, (2010) Letter to the editor, Eur Urol 58; e39-e40.
25. Jentzmik F, Stephan C, Lein M, Miller K, Kamlage B, et al. (2011) Sarcosine in prostate cancer tissue is not a differential metabolite for prostate cancer aggressiveness and biochemical progression. J Urol. 185:706-11.
26. Struys E A, Heijboer A C, van Moorselaar J, Jakobs C, Blankenstein M A. (2010) Serum sarcosine is not a marker for prostate cancer. Ann Clin Biochem. 47:282.
27. Cao D L, Ye D W, Zhu Y, Zhang H L, Wang Y X, Yao X D. (2011) Efforts to resolve the contradictions in early diagnosis of prostate cancer: a comparison of different algorithms of sarcosine in urine. Prostate Cancer Prostatic Dis. 14:166-72.
28. Martinez-Chantar M L, Vazquez-Chantada M, Ariz U. Martinez N, Varela M, et al. (2008) Loss of the glycine N-methyltransferase gene leads to steatosis and hepatocellular carcinoma in mice. Hepatology 47: 1191-1199.
29. Lin J, Lee I M, Song Y, Cook N R, Selhub J, et al. (2010) Plasma homocysteine and cysteine and risk of breast cancer in women. Cancer Res 70: 2397-2405.
30. Khan N, Afaq F, Mukhtar H (2010) Lifestyle as risk factor for cancer: Evidence from human studies. Cancer Lett 293: 133-143.
31. Elshorbagy A K, Nurk E, Gjesdal C G, Tell G S, Ueland P M, et al. (2008) Homocysteine, cysteine, and body composition in the Hordaland Homocysteine Study: does cysteine link amino acid and lipid metabolism? Am J Clin Nutt 88: 738-746.
32. Kane C J, Im R, Amling C L, Presti J C, Jr., Aronson W J, et al. (2010) Outcomes after radical prostatectomy among men who are candidates for active surveillance: results from the SEARCH database. Urology 76: 695-700.
33. Heady J E, Kerr S J (1975) Alteration of glycine N-methyltransferase activity in fetal, adult, and tumor tissues. Cancer Research 35: 640-643.
34. Huang Y C, Lee C M, Chen M, Chung M Y, Chang Y H, et al. (2007) Haplotypes, loss of heterozygosity, and expression levels of glycine N-methyltransferase in prostate cancer. Clin Cancer Res 13: 1412-1420.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.)

1. A system, comprising: cystathionine synthase, cystathionine lyase, and a nanorod, wherein the nanorod comprises two end surfaces and a longitudinal surface.

2. The system of claim 1, wherein the cystathionine synthase is a cystathionine beta-synthase.

3. The system of claim 1, wherein the cystathionine synthase is a polypeptide comprising the sequence as set forth in SEQ ID NO:1 or SEQ ID NO:5.

4. (canceled)

5. The system of claim 1, wherein the cystathionine lyase is a cystathionine gamma-lyase.

6. The system of claim 1, wherein the cystathionine lyase is a polypeptide comprising the sequence as set forth in SEQ ID NO:8 or SEQ ID NO: 12.

7. (canceled)

8. The system of claim 1, wherein the two end surfaces are reactive with cysteine.

9. The system of claim 1, wherein the longitudinal surface is non-reactive with cysteine.

10. The system of claim 1, wherein the nanorod is made of gold, selenium, cadmium, copper, platinum, palladium, or carbon, or a combination thereof.

11. The system of claim 1, wherein the nanorod is single layer carbon nanorod, multilayer carbon nanorod, or ordered mesoporous carbon nanorod.

12. The system of claim 1, wherein the nanorod is a naked nanorod, or a coated nanorod, or a mixture thereof.

13. The system of claim 12, wherein the nanorod is a naked nanorod and the naked nanorod is further protected with CTAB, perylene, or 16-mercaptohexadecyl trimethylammonium bromide (MTAB), or a combination thereof.

14. The system of claim 12, wherein the longitudinal surface of the coated nanorod is coated with platinum, palladium, or selenium, carboxybiphenyl-terminated polystyrene, polystyrene sulfonate (PSS), polyethylene glycol (PEG), methoxy PEG-thiol, carbon, an allotrope of carbon or a combination thereof.

15. (canceled)

16. (canceled)

17. The system of claim 1, further comprising CU²⁺.

18. The system of claim 1, further comprising an isolated sample from a subject.

19. (canceled)

20. (canceled)

21. The system of claim 1, further comprising a PSA test, clinical stage, biopsy Gleason score, pathologic Gleason score, pathologic stage, surgical margin status, lymph node involvement, or seminal vesicle involvement, or a combination thereof.

22. (canceled)

23. A method of detecting a cysteine level in a sample from a subject, comprising:
(a) obtaining a sample from the subject;
(b) processing the sample with cystathionine synthase and cystathionine lyase;
(c) contacting the processed sample with a nanorod;
(d) measuring a change of absorption spectrum of the sample; and
(e) detecting the cysteine level based upon the measured change of absorption spectrum.

24. A nanoelectronic device, comprising:
(a) a first electrode with a first surface;
(b) a second electrode with a second surface;
(c) a hinge connecting the two electrodes, wherein the hinge is non-conductive; and
(d) an ammeter measuring the electric current flowing between the two electrodes,
wherein the two electrodes have different electric potentials;
wherein the first surface is functionalized to bind cysteine, wherein the second surface is not functionalized to bind cysteine, and wherein the two surfaces face each other.

25. A system, comprising: the nanoelectronic device of claim 24, cystathionine synthase, cystathionine lyase, and a linker, wherein the linker has at least one free thiol group, wherein the linker has sufficient length to connect the two surfaces, and wherein the linker is conductive.

26. The system of claim 25, wherein the linker is selected from the group consisting of:
a flexible molecule with inactive and active statuses, wherein the length of the inactive linker is insufficient to connect the two surfaces, and wherein the length of the active linker is sufficient to connect the two surfaces,
a nanoparticle conjugated with a flexible molecule, wherein the length of the inactive linker is insufficient to connect the two surfaces, and wherein the length of the active linker is sufficient to connect the two surfaces,
a cysteine-functionalized nanoparticle, and
a cysteine-bound nanoparticle.

27. (canceled)

28. The system of claim 26, wherein the nanoparticle conjugated with a flexible molecule, the cysteine-functionalized nanoparticle, or the cysteine-bound nanoparticle is a nanorod, nanosphere, nanofiber, nanowire, or nanotube.

29. (canceled)

30. (canceled)

31. (canceled)

32. (canceled)

33. A method of detecting a cysteine level in a sample from a subject, comprising:
(a) obtaining a sample from the subject;
(b) processing the sample with cystathionine synthase and cystathionine lyase;
(c) contacting the processed sample to the nanoelectronic device of claim 24;
(d) removing the processed sample;
(e) contacting a linker with the nanoelectronic device;
(f) measuring the electric current in the nanoelectronic device; and
(g) detecting the cysteine level based upon the measured electric current, wherein the measured electric current is directly or inversely proportional to the cysteine level.

34. A method, comprising:
(a) obtaining a sample from a subject;
(b) processing the sample with cystathionine synthase and cystathionine lyase; and
(c) detecting a cysteine level in the processed sample using an assay to determine cysteine level.

35. (canceled)

36. (canceled)

37. (canceled)

38. The method of claim 34, wherein the sample is urine and the urine cysteine level in the subject is above about 200, 210, 220, 230, or 240 micromoles of cysteine per milligram creatinine.

39. The method of claim 34, wherein the sample is serum and the serum cysteine level in the subject is above about 400, 410, 420, 430, or 440 μM of cysteine.

40. The method of claim 34, wherein the cystathionine synthase is a cystathionine beta-synthase.

41. The method of claim 34, wherein the cystathionine synthase is a polypeptide comprising the sequence as set forth in SEQ ID NO: 1 or SEQ ID NO:5.

42. (canceled)

43. The method of claim 34, wherein the cystathionine lyase is a cystathionine gamma-lyase.

44. The method of claim 34, wherein the cystathionine lyase is a polypeptide comprising the sequence as set forth in SEQ ID NO:8 or SEQ ID NO: 12.

45. (canceled)

46. The method of claim 34, further comprising predicting an increased probability of a recurrence of a cancer in the subject when the detected cysteine level in the subject is higher than a reference cysteine level.

47. (canceled)

48. The method of claim 46, wherein the cancer is prostate cancer, colon cancer, breast cancer, lung cancer, renal cancer, or bladder cancer.

49. The method of claim 46, wherein the reference cysteine level is a mean or median cysteine level in non-recurrent subjects detected by a method, comprising:
(a) obtaining a sample from a subject;
(b) processing the sample with cystathionine synthase and cystathionine lyase; and
(c) detecting a cysteine level in the processed sample using an assay of cysteine level.

50. The method of claim 34, further comprising:
(d) assessing at least one additional parameter, and
(e) predicting an increased probability of a recurrence of a cancer in the subject when the detected cysteine level in the subject is higher than a reference cysteine level and when the additional parameter in the subject is detected to be higher or lower than in non-recurrent subjects.

51. The method of claim 50, wherein the additional parameter is PSA velocity, PSA level, pre-surgical PSA level, post-surgical PSA level, pre-treatment PSA level, post-treatment PSA level, biopsy Gleason score, clinical stage, number of positive cores, number of negative cores, Karnofsky performance status, Hemoglobin value, Lactate dehydrogenase value, Alkaline phosphatase value, Albumin level, urinary albumin level, urinary creatinine level, pre-treatment parameter comprising pretreatment PSA level, pre-treatment biopsy Gleason Score, pre-treatment clinical stage, pre-treatment urinary albumin level, or pre-treatment urinary creatinine level, or a combination thereof.

52. (canceled)

53. (canceled)

54. (canceled)

55. The method of claim 34, further comprising prescribing a first therapy to the subject, when the detected cysteine level in the subject is not higher than a reference cysteine level, or prescribing a second therapy or both the first therapy and the second therapy, when the detected cysteine level in the subject is higher than a reference cysteine level, wherein the first therapy is selected from the group consisting of active surveillance, prostatectomy, HIFU, cryotherapy and radio therapy, and wherein the second therapy is selected from the group consisting of systemic chemotherapy, hormonal therapy, pelvic floor salvage radiation.

56. The method of claim 34, wherein the assay to determine cysteine level comprises using HPLC, GC-MS, a nanorod, a nanoelectronic device, or a system, comprising: cystathionine synthase, cystathionine lyase, and a nanorod.

57. (canceled)

58. (canceled)

59. (canceled)

60. (canceled)

61. The method of claim 34, wherein the assay to determine cysteine level comprises using a system, comprising:
(1) cystathionine synthase;
(2) cystathionine lyase;
(3) a nanoelectronic device, comprising:
(a) a first electrode with a first surface;
(b) a second electrode with a second surface;
(c) a hinge connecting the two electrodes, wherein the hinge is non-conductive; and
(d) an ammeter measuring the electric current flowing between the two electrodes,
wherein the two electrodes have different electric potentials;
wherein the first surface is functionalized to bind cysteine, wherein the second surface is not functionalized to bind cysteine, and wherein the two surfaces face each other; and
(4) a linker, wherein the linker has at least one free thiol group, wherein the linker has sufficient length to connect the two surfaces and wherein the linker is conductive.

62. A polypeptide encoded by the sequence as set forth in SEQ ID NO:2 or SEQ ID NO:9.

63. A polypeptide consisting of the sequence as set forth in SEQ ID NO:5 or SEQ ID NO:12.

64. (canceled)

65. (canceled)
